(19) United States
(12) Patent Application Publication
(10) Pub. No.: US 2026/0306672 A1
(43) Pub. Date: Oct. 8, 2026

(54) TARGETING GPATCH8 FOR TREATING SF3B1-MUTANT CANCERS

(71) Applicants: Fred Hutchinson Cancer Center, Seattle, WA (US); Memorial Sloan Kettering Cancer Center, New York, NY (US)

(72) Inventors: Robert K. Bradley, Seattle, WA (US); Joe Pineda, Seattle, WA (US); Omar Abdel-Wahab, New York, NY (US); Salima Benbarche, New York, NY (US)

(73) Assignees: Fred Hutchinson Cancer Center, Seattle, WA (US); Memorial Sloan Kettering Cancer Center, New York, NY (US)

(21) Appl. No.: 19/483,447

(22) PCT Filed: May 13, 2024
(86) PCT No.: PCT/US2024/029152

§ 371 Date: Nov. 11, 2025

Related U.S. Application Data
(60) Provisional application No. 63/501,600, filed on May 11, 2023.
(60) Provisional application No. 63/608,128, filed on Dec. 8, 2023.

(51) Int. Cl. A61K 48/00; C07K 14/47; C12N 9/22; C12N 15/11; G01N 33/50

(52) U.S. Cl. CPC A61K 48/005; C07K 14/4702; C12N 9/222; C12N 15/111; G01N 33/5023; C07K 2319/43; C07K 2319/95; C12N 2310/20; C12N 2830/42

(57) ABSTRACT

Embodiments of the present disclosure provide methods for treating a subject with cancer, wherein the cancer is characterized by a change-of-function or loss of-function mutation in a recurrently mutated RNA splicing factor gene. In some embodiments, the method can comprise administering to a subject a therapeutically effective amount of a composition that inhibits expression of a trans-acting splicing factor and/or inhibits expression of a cis-acting splicing factor, wherein a cancer-causing error triggered by the change-of-function or loss-of-function mutation in a recurrently mutated RNA splicing factor gene is partially or fully rescued by inhibiting expression of the trans-acting splicing factor and/or inhibiting expression of the cis-acting splicing factor.

### CROSS-REFERENCES TO RELATED APPLICATIONS

This application claims the benefit of U.S. Provisional Application No. 63/501,600, filed May 11, 2023, and U.S. Provisional Application No. 63/608,128, filed Dec. 8, 2023, the disclosures of which are incorporated herein by reference in their entirety.

### STATEMENT OF GOVERNMENT LICENSE RIGHTS

This invention was made with government support under HL128239, CA251138, and HL151651, awarded by the National Institutes of Health. The government has certain rights in the invention.

### STATEMENT REGARDING SEQUENCE LISTING

The Sequence Listing XML associated with this application is provided in XML format and is hereby incorporated by reference into the specification. The name of the XML file containing the sequence listing is 1896-P89WO_Seq_List_20240513.xml. The XML file is 86,080 bytes; was created on May 13, 2024; and is being submitted electronically via Patent Center with the filing of the specification.

### BACKGROUND

The discovery of frequent mutations in genes encoding components of the RNA splicing machinery was among the most unexpected findings from genomic sequencing efforts. Mutations in RNA splicing factors are the single most common class of mutations in patients with myelodysplastic syndromes (MDS) and also highly recurrent in acute myeloid leukemia (AML), chronic lymphocytic leukemia (CLL), and non-mucosal forms of melanoma. (Yoshida et al., Nature 478:64-69, 2011; Papaemmanuil et al., N. Engl. J. Med. 365:1284-1395, 2011; Wang et al., N. Engl. J. Med. 365:2497-2506, 2011; Quesada et al., Nat. Genet. 44:47-52, 2012; Alsafadi et al., Nat. Commun. 7:10615. doi: 10.1038/ncomms10615, 2016; Furney et al., Cancer Discov. 3:1122-1129, 2013). These same mutations are also seen in 2-5% of patients with breast cancer and non-small cell lung cancer as well as other common solid cancers. (Liu et al., J. Clin. Invest. 131. 10.1172/JCI138315, 2021; Imielinski et al. Cell 150:1107-1120, 2012). Of the RNA splicing factors recurrently mutated in cancer, mutations altering the core RNA splicing factor SF3B1 are the most prevalent. (Rahman et al., Cell 180:208-208.el. doi: 10.1016/j.cell.2019.12.011, 2020)

Spliceosomal mutations alter splice site and exon recognition to cause dramatic mis-splicing of a restricted set of genes while leaving most genes unaffected. Recent studies have identified that the most common spliceosomal mutations alter RNA recognition in a sequence-specific manner to cause widespread RNA mis-splicing of many genes in a manner distinct from loss of function. Of the RNA splicing factors recurrently mutated in cancer, mutations altering the core RNA splicing factor SF3B1 are the most prevalent. For example, cancer-associated mutations affecting SF3B1 occur as hotspot mutations that induce usage of cryptic (normally ignored) 3′ splice sites. In contrast to these sequence-specific changes in splice site recognition, deletion of SF3B1 results in splicing failure characterized by widespread cassette exon skipping and intron retention.

The frequency and neomorphic nature of RNA splicing factor mutations in combination with the need for additional mutationally informed therapies for myeloid malignancies has made these genetic alterations exciting candidates for therapeutic targeting. Interestingly, mutations in the most commonly mutated RNA splicing factors in myeloid malignancies occur as heterozygous mutations and in a statistically significant, mutually exclusive manner with one another. It has been demonstrated that this mutual exclusivity occurs because cells bearing spliceosomal mutations are genetically dependent on otherwise wild-type (WT) splicing catalysis for cell survival. This genetic observation motivated preclinical studies identifying that diverse compounds that modulate global RNA splicing (e.g., that alter splicing in both spliceosome-mutant and WT cells) preferentially kill spliceosome-mutant cells. Several of these agents have since entered clinical trials for MDS patients, including SF3b binding agents (e.g., H3B-8880), protein arginine methyltransferase (PRMT) inhibitors, and RBM39 degrading agents.

Although these therapies have notable potential, no current approaches discriminate between WT versus mutant splicing proteins—they all perturb splicing in both WT and spliceosome-mutant cells—and so their therapeutic indices are not yet defined. This is an important concern given the essentiality of RNA splicing in healthy cells.

Consequently, developing a reliable and generalizable method to permit expression of a given gene or protein in cancer cells, but not normal cells, or alternately in normal cells but not cancer cells, as well as identifying therapeutic targets critical for aberrant splicing would be a major and important step toward bringing gene therapy for cancers into the clinic. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present disclosure provides for a method of treating a subject with cancer. In some embodiments, the cancer is characterized by a change-of-function or loss-of-function mutation in a recurrently mutated RNA splicing factor gene. In some embodiments, the method comprises administering to a subject a therapeutically effective amount of a composition that inhibits expression of a trans-acting splicing factor and/or inhibits expression of a cis-acting splicing factor, wherein a cancer-causing error triggered by the change-of-function or loss-of-function mutation in a recurrently mutated RNA splicing factor gene is partially or fully rescued by inhibiting expression of the trans-acting splicing factor and/or inhibiting expression of the cis-acting splicing factor. In still another embodiment, the method comprises administering to a subject a therapeutically effective amount of a composition that inhibits function of a trans-acting splicing factor and/or inhibits function of a cis-acting splicing factor, wherein a cancer-causing error triggered by the change-of-function or loss-of-function mutation in a recurrently mutated RNA splicing factor gene is partially or fully rescued by inhibiting function of the trans-acting splicing factor and/or inhibiting the function of the cis-acting splicing factor.

In some embodiments, the recurrently mutated RNA splicing factor gene comprises SF3B1. In some embodiments, the recurrent change-of-function mutation in SF3B1 results in an amino acid substitution that comprises E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, 1704F, G740E, G742D, A762V, Y765C, D781E, D781G, M7841, E802Q, M971T, M971V, or combinations thereof, with reference to the wild-type amino acid sequence set forth in SEQ ID NO:1.

In some embodiments, the cancer is selected from myelodysplastic syndromes (MDS), a chronic myelomonocytic leukemia (CMML), a chronic lymphocytic leukemia (CLL), an acute myeloid leukemia (AML), an uveal melanoma, a mucosal melanoma, a skin melanoma, a breast cancer, a pancreatic cancer, an endometrial cancer, a liver cancer, a lung cancer, a mesothelioma, or other cancers with recurrent SF3B1 mutations.

In some embodiments, the composition comprises a nucleic acid construct that inhibits expression of the trans-acting splicing factor and/or inhibits expression of the cis-acting splicing factor by attenuating the expression of a target gene, wherein the trans-acting splicing factor and/or the cis-acting splicing factor are encoded by the target gene. In some embodiments, the nucleic acid construct used to inhibit gene expression includes but is not limited to antisense oligonucleotides, gene knockout via CRISPR, gene knockout via CRISPRi, or RNA-targeting CRISPR.

In yet another embodiment, the composition comprises at least one protein-binding moiety that binds to at least one protein of interest and at least one tag that promotes degradation of the at least one protein of interest, wherein the at least one protein of interest comprises the trans-acting splicing factor and/or the cis-acting splicing factor. In some embodiments, the composition comprises a small molecule that disrupts a protein-protein interaction required for the trans-acting splicing factor and/or the cis-acting splicing factor to recognize and bind to its splice site. In some embodiments, the protein-binding moiety and the tag can comprise proteolysis-targeting chimera (PROTAC) molecules. In other embodiments, molecular glue is used to create an interaction between the protein of interest and a second protein, wherein the interaction between the protein of interest and the second protein results in the degradation of the protein of interest.

In still other embodiments, the composition comprises a small molecule that disrupts a protein-protein interaction required for the trans-acting splicing factor and/or the cis-acting splicing factor to recognize and bind to its splice site. In some embodiments, the small molecule binds to a trans-acting splicing factor and/or a cis-acting splicing factor, which can block binding of the trans-acting splicing factor and/or the cis-acting splicing factor to at least one facilitating protein, wherein the trans-acting splicing factor and/or the cis-acting splicing factor cannot bind to its splice site without first binding to the facilitating protein. In other embodiments, the small molecule binds to at least one facilitating protein, which blocks binding of the facilitating protein to the trans-acting splicing factor and/or the cis-acting splicing factor, wherein the trans-acting splicing factor and/or the cis-acting splicing factor cannot bind to its splice site without first binding to the facilitating protein.

In some embodiments, the trans-acting splicing factor comprises a protein encoded by a GPATCH8 gene. In some embodiments, the protein encoded by the GPATCH8 gene is a G patch domain-containing protein 8 (GPATCH8) protein.

In some embodiments, the therapeutically effective amount of the composition inhibits expression of the GPATCH8 protein by at least 25% (e.g., 25%, 30%, 35%, 40%, 45%, 50%, 55%, 60%, 65%, 70%, 75%, 80%, 85%, 90%, 95%, or 100%). In some embodiments, the GPATCH8 protein cannot bind to its 3′ recognition splice site if GPATCH8 protein expression is reduced by at least 25% (e.g., 25%, 30%, 35%, 40%, 45%, 50%, 55%, 60%, 65%, 70%, 75%, 80%, 85%, 90%, 95%, or 100%) thereby partially or fully rescuing mis-splicing caused by the mutation in a recurrently mutated RNA splicing factor gene.

In still other embodiments, the therapeutically effective amount of the composition inhibits function of the GPATCH8 protein by at least 25% (e.g., 25%, 30%, 35%, 40%, 45%, 50%, 55%, 60%, 65%, 70%, 75%, 80%, 85%, 90%, 95%, or 100%). In some embodiments, the GPATCH8 protein cannot bind to its 3′ recognition splice site if GPATCH8 protein function is reduced by at least 25% (e.g., 25%, 30%, 35%, 40%, 45%, 50%, 55%, 60%, 65%, 70%, 75%, 80%, 85%, 90%, 95%, or 100%), thereby partially or fully rescuing mis-splicing caused by the mutation in a recurrently mutated RNA splicing factor gene.

In some embodiments, the composition further comprises a therapeutic agent.

In another aspect, the disclosure provides for an in vitro method to screen for a trans-acting splicing factor and/or a cis-acting splicing factor required for mis-splicing in a change-of-function or loss-of-function mutation in a recurrently mutated RNA splicing factor gene. In some embodiments, the method comprises: (a) generating an expression cassette comprising a coding sequence (CDS) interrupted by at least one artificial nucleic acid intron, a promoter operatively linked to the CDS, wherein upon splicing of the artificial nucleic acid intron the CDS encodes a detectable reporter protein in wild-type cells and wherein the CDS does not encode a detectable reporter protein in cells comprising the recurrently mutated RNA splicing factor gene; (b) contacting the construct generated in (a) to a population of cells; (c) performing a positive enrichment CRISPR screen to identify at least one gene (i.e., gene of interest) whose knockout enhances expression of the detectable reporter protein in cells comprising the recurrently mutated RNA splicing factor gene; and (d) detecting expression of the detectable reporter protein, wherein expression of the detectable reporter protein in cells comprising the recurrently mutated RNA splicing factor gene is increased if the gene knockout is required for mis-splicing in recurrently mutated RNA splicing factor gene.

In some embodiments, the artificial nucleic acid intron comprises: a 5′ splice site; a canonical 3′ splice site; at least one cryptic 3′ splice site, that is within about 100 nucleotides upstream of the canonical 3′ splice site or within about 50 nucleotides downstream of the canonical 3′ splice site; a pyrimidine-rich domain comprising at least 6 consecutive nucleotides, wherein the sequence of the pyrimidine-rich domain is at least 60% pyrimidine nucleotides, and wherein the pyrimidine-rich domain is within at least 50 nucleotides of a cryptic 3′ splice site; or at least one branchpoint at least 15 nucleotides upstream of the canonical 3′ splice site.

In some embodiments, the intron is at least about 50 nucleotides to about 1000 nucleotides in length.

In some embodiments, the intron is derived from a human wildtype intron comprising intron 1 of MTERFD3, intron 4 of MYO15B, intron 10 of SYTL1, intron 11 of SYTL1, intron 4 of MAP3K7, intron 1 of ORAI2, or intron 1 of TMEM14C. In still other embodiments, the human wildtype intron from which the intron is derived can comprise: intron 1 of MTERFD3 comprising a sequence set forth in SEQ ID NO:2; intron 4 of MYO15B comprising a sequence set forth in SEQ ID NO:3; intron 10 of SYTL1 comprising a sequence set forth in SEQ ID NO:4; intron 11 of SYTL1 comprising a sequence set forth in SEQ ID NO:5; intron 4 of MAP3K7 comprising a sequence set forth in SEQ ID NO:6; intron 1 of ORAI2 comprising a sequence set forth in SEQ ID NO:7; or intron 1 of TMEM14C comprising a sequence set forth in SEQ ID NO:8.

In some embodiments, the intron is derived from a human wildtype intron 4 of MAP3K7, and wherein the intron further comprises one, two, three, or more of the following features: a 5′ splice site comprising a GT dinucleotide immediately followed by a consensus 5′ splice site context, optionally wherein the consensus 5′ splice site context can comprise AAG, GAG, or GTG; a canonical 3′ splice site that can comprise an AG dinucleotide immediately preceded by a C or T; at least one cryptic 3′ splice site, located at least 5 nucleotides upstream of the canonical 3′ splice site, with an AG dinucleotide and comprising a sequence that is a weaker 3′ splice site than is the canonical 3′ splice site, where splice site strength is estimated with the MaxEntScan algorithm or similar methods; a pyrimidine-rich domain that can comprise at least 15 consecutive nucleotides, wherein the sequence of the pyrimidine-rich domain can be at least 60% pyrimidine nucleotides and at least 40% thymine nucleotides, and wherein the pyrimidine-rich domain can be within at least 30 nucleotides of a cryptic 3′ splice site; or at least one branchpoint at least 20 nucleotides upstream of the canonical 3′ splice site.

In some embodiments, the intron has a 5′ end domain with about 10 to about 150 nucleotides having at least 50% sequence identity to a sequence of the 5′-most 10 to about 150 nucleotides of the wildtype intron.

In some embodiments the intron has a 3′ end domain with about 50 to about 350 nucleotides having at least 50% sequence identity to a sequence of the 3′-most 50 to about 350 nucleotides of the wildtype intron.

In some embodiments, the intron has a sequence with at least 75% sequence identity to a sequence selected from SEQ ID NOS: 9-28.

In some embodiments, the 5′ splice site comprises a sequence selected from GTGAG, GTAAG, GTGCG, GTACG, GTGGG, GTAGG, GTGTG, GTATG, or GTATC.

In some embodiments, the canonical 3′ splice site comprises a sequence selected from AAG, CAG, or TAG.

In some embodiments, the at least one cryptic 3′ splice site comprises a sequence selected from AAG, CAG, GAG, TAG, ATG, CTG, GTG, or TTG.

In some embodiments, the intron comprises a plurality of cryptic 3′ splice sites within about 100 nucleotides upstream of the canonical 3′ splice site or within about 100 nucleotides downstream of the canonical 3′ splice site, and wherein each of the plurality of the cryptic 3′ splice sites comprises a sequence independently selected from AAG, CAG, GAG, TAG, ATG, CTG, GTG, or TTG.

In some embodiments, the pyrimidine-rich domain is characterized by one, two, three, or all of the following: wherein the pyrimidine-rich domain comprises at least 15 consecutive nucleotides; wherein the pyrimidine-rich domain has a sequence with at least 60% pyrimidine nucleotides and is at least 40% thymine nucleotides; wherein the pyrimidine-rich domain is within at least 30 nucleotides of a cryptic 3′ splice site; or wherein the pyrimidine-rich domain has a sequence with at least 50% sequence identity to any 20 nucleotides selected from the sequence set forth as SEQ ID NO:29.

In some embodiments, the at least one branchpoint is at least 20 nucleotides upstream of the canonical 3′ splice site, and wherein the branchpoint nucleotide is an adenine.

In some embodiments, the branchpoint and surrounding sequence context has sequence identity of at least 60% to the sequence tactaAca, where the uppercase A is the branchpoint nucleotide.

In some embodiments, the intron is configured to be spliced differently in a cancer cell comprising a change-of-function or loss-of-function mutation in a recurrently mutated RNA splicing factor gene relative to the splicing pattern of the intron in a cell lacking a change-of-function or loss-of-function mutation in a recurrently mutated RNA splicing factor gene.

In some embodiments, the RNA splicing factor gene is SF3B1. In still other embodiments, the recurrent change-of-function mutation in SF3B1 results in an amino acid substitution comprising E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, I704F, G740E, G742D, A762V, Y765C, D781E, D781G, M784I, E802Q, M971T, M971V, or combinations thereof, with reference to the wild-type amino acid sequence set forth in SEQ ID NO: 1.

In some embodiments, the artificial nucleic acid construct further comprises a first exon domain and a second exon domain, wherein the intron is disposed between the first exon domain and the second exon domain. In some embodiments, the combination of the first exon domain and the second exon domain without the intron encodes part or all of a protein of interest.

In some embodiments, the nucleic acid intron construct comprises an expression cassette comprising the first exon domain, the intron, the second exon domain, and a promoter sequence operatively linked thereto.

In some embodiments, detecting expression of the detectable reporter protein comprises quantifying the amount of the reporter protein. In certain embodiments, the reporter protein comprises a fluorescent or luminescent protein.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1I shows that synthetic introns reveal trans-acting factors that are required for mutant S-dependent mis-splicing. (FIG. 1A) Schema of synthetic intron fluorescent reporter that uses a MAP3K7-derived synthetic intron to report mutant SF3B1 splicing activity. Aberrant 3′ splice site (3′ss) usage in SF3B1 mutant cells results in reduced fluorescent protein expression than in SF3B1 wild-type (WT) cells. (FIG. 1B) Histogram of mEmerald/mCardinal fluorescent protein expression in isogenic MCF10A cells with or without knockin of heterozygous SF3B1^{K700E} mutation and stable expression of the MAP3K7 synthetic intron reporter from (FIG. 1A). (FIG. 1C) Schema of whole-genome CRISPR screen from cells in (FIG. 1B) evaluating enrichment of sgRNAs in top 10% and bottom 10% mEmerald/mCardinal expressing cells. (FIG. 1D) Relative per-gene enrichment in SF3B1-mutant versus WT cells from a whole-genome CRISPR knockout screen to identify genes whose loss corrects mis-splicing in SF3B1-mutant cells using the reporter in (FIG. 1A). (FIG. 1E) Normalized log 2 sgRNA read counts in bottom versus top 10% mEmerald/mCardinal expressing cells for key genes from (FIG. 1D). (FIG. 1F) RT-PCR validating that GPATCH8 knockout antagonizes SF3B1^{K700E} mutant-dependent mis-splicing of both the synthetic intron (top) and endogenous MAP3K7 (bottom). (FIG. 1G) As in (FIG. 1F) for endogenous MAP3K7 utilizing uveal melanoma (MEL270) cells with or without SF3B1^{K700E} mutation, AML (K562) cells with or without endogenous SF3B1^{K666N/WT} mutations, pancreatic cancer (Panc05.04) cells with endogenous SF3B1^{K700E/WT} mutation, and uveal melanoma (MEL202) cells with endogenous SF3B1^{R625H/WT} mutation. (FIG. 1H) As in (FIG. 1B) but with or without GPATCH8 knockout. (FIG. 1I) Western blot of MAP3K7 protein in MCF10A cells from (FIG. 1H) and MEL202 cells from (FIG. 1G).

FIGS. 2A-2G show antagonistic effects of SUGP1 versus GPATCH8 deletion on SF3B1 mutation induced aberrant RNA splicing. (FIG. 2A) Representative flow cytometry analysis evaluating impact of SUGP1 versus GPATCH8 deletion on splicing of the MAP3K7-derived synthetic intron reporter as demonstrated by mCardinal versus mEmerald fluorescence. Experiment performed in K562 cells with or without knockin of heterozygous SF3B1^{K700E} mutation and stable CRISPR-Cas9 knockout (KO) of GPATCH8 or SUGP1 as indicated. (FIG. 2B) Number and types of splicing events altered in K562 cells from (FIG. 2A) by genome-wide RNA-seq analysis. Each mutant condition was compared to sgControl. (FIG. 2C) Scatter plot of isoform expression values in SF3B1^{K700E} mutant cells compared to SUGP1 KO cells (left) and bar plot indicating proportion of splicing events in SF3B1^{K700E} mutant cells that are concordant with those in SUGP1 KO cells across two biological replicates. Correlation coefficient (p) and p-value estimated via Pearson's correlation. (FIG. 2D) Scatter plot of splicing events in SF3B1^{K700E} mutant cells compared to GPATCH8 KO cells (left) and bar plot indicating proportion of splicing events in SF3B1^{K700E} mutant cells that require GPATCH8 across two biological replicates. (FIG. 2E) RT-PCR of splicing events regulated by mutant SF3B1 and/or influenced by deletion of SUGP1 or GPATCH8. RT-PCR of MAP3K7 synthetic intron and endogenous MAP3K7, PPM1M, SMURF2, and ZDHCC16 events are shown. Experiment performed in K562 cells with or without knockin of heterozygous SF3B1^{K700E} mutation and stable CRISPR-Cas9 KO of GPATCH8 or SUGP1 as indicated. (FIG. 2F) Sequence logos of the 5′ splice site (circle), branchpoint sequence (square), and 3′ splice site (triangle) surrounding cassette exons skipped or included upon GPATCH8 deletion in SF3B1 WT K562 cells. (FIG. 2G) Branchpoint (BP) positions relative to the 3′ splice site in introns flanking cassette exons in skipped or included upon GPATCH8 deletion. Impact of GPATCH8 deletion on RNA splicing and aberrant RNA splicing induced by mutant SF3B1.

FIGS. 3A through 3E show GPATCH8 and SUGP1 bind intronic sequences in pre-mRNA and the G-patch domains of each protein are required for splicing regulation. TRIBE-seq analysis of SUGP1 (blue) and GPATCH8 (green) guided RNA editing sites in HEK 293T cells showing (FIG. 3A) number of edit sites and (FIG. 3B) edited sites of SUGP1 relative to 3′ splice site. (FIG. 3C) Schema of the experimental design for CRISPR/Cas9-tiling scan of the protein coding domains of GPATCH8 and SUGP1. (FIG. 3D) Normalized CRISPR score of each sgRNA construct (dots) and the smoothed score (line) of the SUGP1 sgRNA tiling screen. Data represents the average of a duplicate experiment. (FIG. 3E) As in (FIG. 3D) but for GPATCH8.

FIGS. 4A-4D show GPATCH8's G-patch domain is required for splicing regulation and is not interchangeable with SUGP1's G-patch domain. (FIG. 4A) Sequence alignment of the G-patch domains of SUGP1 (SEQ ID NO:72), GPATCH8 (SEQ ID NO: 73), and GPATCH11 (SEQ ID NO:74). Invariable residues and positions with 70% similarity are highlighted in dark and light pink, respectively. The consensus sequence is shown in red below the alignment with small letters denoting a—aromatic, h—hydrophobic, l—aliphatic, s—small, and u—tiny amino acids. Arrows indicate the Glycine residues mutated in the indicated cDNAs in (FIG. 4B). (FIG. 4B) Diagram of the full-length GPATCH8 and SUGP1 and mutations made in each cDNA including deletion of the C-terminus of GPATCH8, mutation of the 2^{nd} and 5^{th} conserved Glycine residues within the G-patch domain of each protein, and versions of each protein where the G-patch domain of GPATCH8 and SUGP1 were exchanged. (FIG. 4C) Bar graph of % mEmerald⁺ cells in SF3B1^{K700E}/WT knockin K562 cells containing the MAP3K7 synthetic intron interrupting mEmerald's coding sequence. The impact of knockout (KO) of GPATCH8 followed by introduction of various GPATCH8/SUGP1 cDNAs on mEmerald fluorescence is shown. Measurements are from three independent transductions (Mean±s.d., one-way ANOVA with Dunnett's for multiple comparison with sgRenilla+control cDNA as reference, ****p<0.0001). (FIG. 4D) FACS plots representation of mCardinal/mEmerald fluorescence in a portion of the groups from (FIG. 4C). Comparison of the interaction between GPATCH8 N terminus and SF3B1 heat domain and SUGP1 C terminus and SF3B1 heat domain.

FIGS. 5A through 5C show GPATCH8 interacts with DHX15 through its G-Patch domain. (FIG. 5A) Western blot of N-terminally 3×-FLAG tagged endogenous GPATCH8 in K562 cells with or without SF3B1^{K700E} heterozygous knockin and with or without GPATCH8 knockout. (FIG. 5B) Volcano plot of proteins enriched with anti-FLAG versus mouse IgG1 isotype control immunoprecipitation in cells from (FIG. 5A) followed by mass spectrometry. Measurements are from three independent immunoprecipitations. Unpaired t-test was used to calculate p-value in differential analysis, volcano plot was generated based on log₂ fold change and q-value (multiple testing corrected p-value). A q-value of ≤0.05 was considered the statistically significant cut-off. (FIG. 5C) Overlay of published structure of SUGP1 (dash single dot) in complex with DHX15 (dash double dot) with alpha-fold predicted structure of GPATCH8 and DHX15 interaction (short dash and long dash, respectively). Inset shows conserved amino acid residues between G-patch domains of SUGP1 and DHX15.

FIGS. 6A-6E show suppression of GPATCH8 rescues hematopoietic defects of SF3B1 mutant hematopoietic cells. (FIG. 6A) Schema of anti-Gpatch8 shRNA experiments in hematopoietic precursor cells from Sf3b1 wild-type (WT) and mutant mice. (FIG. 6B) Photograph of methylcellulose colonies generated from hematopoietic precursor cells of Sf3b1 WT and mutant mice with or without suppression of Gpatch8. (FIG. 6C) Enumeration of numbers and types of hematopoietic colonies from (B). Measurements are from three independent experiments (Mean±s.d., unpaired two-sided Student t test, *p<0.0332, **p<0.0021, ***p<0.0002). (FIG. 6D) FACS plot of live, BFP⁺ cells followed by CD71/Glycophorin A (GlyA) FACS staining of one representative CD34⁺ cord blood donor treated with sgRNAs to create the SF3B1^{K700E}/WT mutation or SF3B1^{K700K} control along with knockout of GPATCH8 (or AAVSI negative control). (FIG. 6E) Absolute numbers of BFP⁺ GlyA⁺ cells at day 7 following erythroid culture from three separate donors. Mean±SD. Two-way ANOVA.

FIGS. 7A-7H show the impact of GPATCH8 and SUGP1 deletion on RNA splicing and aberrant RNA splicing induced by mutant SF3B1. (FIG. 7A) RNA-sequencing coverage plots of MAP3K7 aberrant 3′ splice site usage in SF3B1^{K700E/WT} knockin MCF10A cells and the impact of GPATCH8 deletion on this event. (FIG. 7B) Impact of GPATCH8 deletion on aberrant splicing of MAP3K7-derived synthetic intron reporter in SF3B1^{K666N/WT} K562 cells stably expressing the reporter as demonstrated by FACS plots of mCardinal versus mEmerald fluorescence. Data from SF3B1^{WT} and SF3B1^{K700E/WT} K562 cells are shown on left for reference. (FIG. 7C) Histogram of mEmerald/mCardinal fluorescent protein expression in isogenic MEL270 cells with or without SF3B1^{K700E} mutation and MEL202 cells (which contain a naturally occurring SF3B1R625H/WT mutation) expressing the MAP3K7-derived synthetic intron shown in FIG. 1A. (FIG. 7D) As in (FIG. 7A) but using MEL202 cells with or without GPATCH8 knockout (KO). (FIG. 7E) Bar plot of percentage of Emerald⁺ cells from FIG. 3B in SF3B1^{WT} versus SF3B1^{K700E/WT} mutant K562 cells. Experiment performed in biological triplicate and mean±SD shown. Comparison performed by paired t-test. (FIG. 7F) RT-PCR of K562 cells with SF3B1^{K700E} or SF3B1^{K666N} mutant knockin and with deletion of GPATCH8 in the SF3B1^{K666N} mutant cells. (FIG. 7G) Scatter plot of PSI values comparing biologic replicates of RNA-seq data from K562 cells with or without SF3B1^{K700E} mutation and GPATCH8 or SUGP1 KO. Pearson's correlation coefficient (p). (FIG. 7H) Upset plot of unique and shared splicing changes across replicates in the cells from (FIG. 7C) compared between the various cell genotypes shown.

FIGS. 8A-8G show impact of GPATCH8 and SUGP1 on RNA splicing, alone and in the presence of mutant SF3B1 in K562 and MCF10A cells. (FIG. 8A) Scatter plot of altered 3′ splice site usage (top row), retained introns (middle row), and cassette exon skipping (bottom row) in the RNA-seq data from K562 cells with or without SF3B1^{K700E} mutation and knockout (KO) of GPATCH8 or SUGP1. Each mutant cell type is indicated in the top column and compared with SF3B1/GPATCH8/SUGP1 wild-type cells. (FIG. 8B) RNA-seq coverage plots of MCF10A cells with SF3B1^{K700E} mutation and/or GPATCH8 knockout (KO) at SMURF2, PPM1M, and ZDHHC16. (FIG. 8C) Scatter plot of isoform expression values in GPATCH8 KO compared to GPATCH8 WT in the mutant SF3B1^{K700E} background and bar plot (right) indicating proportion of splicing events in SF3B1^{K700E} mutant cells that are corrected upon GPATCH8 deletion across two biological replicates. (FIG. 8D) Spatial purine/pyrimidine enrichment plots along cassette exons differentially spliced after GPATCH8 KO in MCF10 cells (top). YYYYY (SEQ ID NO:75); RRRRR (SEQ ID NO: 76) Bottom: Sequence logo plots of the upstream and downstream 5′ splice sites (5′ ss) of differentially spliced cassette exons upon GPATCH8 KO. Arrow indicates increased preference for As at the +3 position of the downstream 5′ ss. (FIG. 8E) As in (FIG. 8D) but evaluating the upstream and downstream 3′ splice site regions of differentially spliced cassette exons upon GPATCH8 KO. (FIG. 8F) Sequence logos of the 5′ splice site (circle), branchpoint sequence (square), and 3′ splice site (triangle) surrounding cassette exons skipped or included upon SUGP1 deletion in SF3B1 WT K562 cells. (FIG. 8G) Location of the branchpoint (BP) position relative to the 3′ splice site in the upstream and downstream introns in the comparisons from (FIG. 8F); p-value estimated via a two-sided Mann-Whitney U-test.

FIGS. 9A-9E show evaluation of SUGP1 and GPATCH8 RNA binding sites by TRIBE-seq and expression of mutant SUGP1 or GPATCH8 G-Patch domain mimics SF3B1^{K700E} induced mis-splicing in SF3B1^{WT} cells. (FIG. 9A) Correlation of HyperTRIBE editing sites from biological duplicate RNA-seq data generated by GPATCH8-ADAR (left) and ADAR-SUGP1 (right) constructs. Two tailed Pearson R correlation shown. (FIG. 9B) SUGP1 (upper graph) and GPATCH8 (lower two graphs) TRIBE-seq edit sites relative to the indicated splice sites. (FIG. 9C) Correlation of biological duplicate GPATCH8 (top) and SUGP1 (bottom) CRISPR domain scan sgRNA screen data. Pearson correlation coefficient is shown. (FIG. 9D) Bar graph of % mEmerald⁺ cells in SF3B1 wild-type (WT) K562 cells containing the MAP3K7 synthetic intron interrupting mEmerald's coding sequence. The impact of knockout (KO) of GPATCH8 followed by introduction of various GPATCH8/SUGP1 cDNAs on mEmerald fluorescence is shown. Measurements are from three independent transductions (Mean±s.d., one-way ANOVA with Dunnett's for multiple comparison with sgRenilla+control cDNA as reference, *p<0.0332, ****p<0.0001). (FIG. 9E) FACS plots representation of mCardinal/mEmerald fluorescence in a portion of the groups from (FIG. 9D).

FIGS. 10A-10E show generation of N-terminal GPATCH8 3×-FLAG knockin cells and GPATCH8 interacting proteins. (FIG. 10A) Schema of vector engineered for CRISPR HDR-mediated knockin of sequence encoding an N-terminal 3×-FLAG tag into the GPATCH8 locus in human AML cell line K562 wild-type (WT) or mutant for SF3B1^{K700E} (FIG. 10B) Genomic DNA PCRs reveal insertion of sequence encoding 3×-FLAG epitope. (FIG. 10C) Sanger sequencing electropherograms of bands (GPATCH8 parental sequence SEQ ID NO:77) from the PCR in (FIG. 10A) revealing 3×-FLAG sequence in SF3B1^{WT} K562 cells (SEQ ID NO:78 and SEQ ID NO:79). (FIG. 10D) FLAG Western blot of an N-terminal 3×-FLAG tag into the GPATCH8 locus in K562 wild-type or mutant for SF3B1^{K700E} (FIG. 10E) Gene ontology (GO) enrichment analysis of GPATCH8 interacting proteins following FLAG immunoprecipitation/mass spectrometry analysis of cells from (FIG. 10D). One-sided Fisher's exact test was performed. Top 20 pathways shown with adjusted p-value<0.01 (adjusted by Benjamini-Hochberg method for multiple test correction).

FIGS. 11A-11F show generation and validation of Sfb31^{K666N} and Sf3b1^{R625H} conditional knockin mice. (FIG. 11A) Schema of Sf3b1 alleles used to generate Sfb31^{K666N} and Sf3b1^{R625H} conditional knockin mice. Both the Sfb37^{K666N} mice and Sf3b1^{R625H} mice were generated using an inverted minigene of the indicated exons of Sf3b1 with the respective mutation included knocked into the endogenous Sf3b1 locus. (FIG. 11B) Genomic DNA PCR of embryonic stem cell clones of the Sf3b1^{K666N} mice using PCR primers indicated below each gel (location of which is shown in (FIG. 11A). The numbers above each gel indicate the ES cell clone number (or control or ladder). (FIG. 11C) Southern blot of ES cells from (FIG. 11B) for external long arm probe (PB1) (the location of which is shown in (FIG. 11A)). (FIG. 11D) As in (FIG. 11C) but using PB2 probe. (FIG. 11E) Genomic DNA PCR of embryonic stem cell clones of the Sf3b1^{R625H} mice using PCR primers indicated below each gel (location of which is shown in (FIG. 11A)). The numbers above each gel indicate the ES cell clone number (or control or ladder). (FIG. 11F) Sanger sequencing electropherograms of cDNA from blood cells of Mx1-cre Sf3b1 wild-type (WT), Mx1-cre Sf3b1^{K700E/WT}, Mx1-creSf3b1^{K666N/WT}, and Mx1-creSf3b1^{R625H/WT} mice 4 weeks after treatment with plpC.

FIGS. 12A-12C show GPATCH8 regulated splicing of Map3k7 in Sf3b1 mutant mice. (FIG. 12A) qRT-PCR (top) of Gpatch8 expression normalized to Gapdh and RT-PCR (bottom) of endogenous Map3k7 in hematopoietic precursor cells of Sf3b1 wild-type (WT) and mutant mice with or without suppression of Gpatch8. Measurements are from three independent experiments (Mean±s.d., unpaired two-sided Student t test comparing each GPATCH8 shRNA to the NTC shRNA in corresponding Sf3b1 genotype, **p<0.002, ***p<0.0002, ****p<0.0001). (FIG. 12B) FACS plot of live, BFP⁻ cells of two additional representative CD34⁺ cord blood donor treated with sgRNAs to create the SF3B1^{K700E/WT} mutation or SF3B1^{K700K} control along with knockout of GPATCH8 (or AAVSI negative control). (FIG. 12C) Sanger sequencing electropherograms at editing site of GPATCH8 sgRNAs demonstrating GPATCH8 frameshift (SEQ ID NO:80) in GPATCH8 knockout cells. AAVSI knockout sequence SEQ ID NO:81).

### DETAILED DESCRIPTION

Many cancers carry recurrent mutations in RNA splicing factor genes, or “spliceosomal mutations,” which induce sequence-specific changes in RNA splicing. In this usage, “cancer” may refer to any dysplastic disease, neoplastic disease, or other disease characterized by disordered cell differentiation, insufficient cell production, impaired cell death, or accelerated cell proliferation. These diseases include solid tumors, malignant ascites, myelodysplastic syndromes, leukemias, lymphomas, and other malignancies and disorders of the bone marrow and hematopoietic system, bone marrow failure syndromes, connective tissue malignancies, metastatic disease, minimal residual disease following transplantation of organs or stem cells, multi-drug resistant cancers, primary or secondary malignancies, angiogenesis related to malignancy, or other forms of cancer. For example, SF3B1 is the most mutated splicing factor gene. SF3B1 mutations occur in many cancers, including myelodysplastic syndromes (MDS), chronic lymphocytic leukemia (CLL), uveal melanoma, mucosal melanoma, skin melanoma, breast cancer, pancreatic cancer, and others. The inventors of the present disclosure have previously demonstrated that SF3B1 and other common splicing factor mutations cause highly specific changes in RNA splicing mechanisms, such that cancer cells carrying mutations in SF3B1 or other RNA splicing factors do or do not efficiently remove introns with particular sequences.

Mutations in SF3B1 occur as heterozygous gain of function point mutations affecting specific amino acid residues concentrated in the HEAT repeat domains 4-7 of SF3B1. A number of prior studies delineated that cancer-associated SF3B1 mutations globally alter RNA splicing in a manner distinct from SF3B1 loss and result in widespread use of aberrant branchpoint nucleotides. (Darman et al., Cell Rep. 13:1033-1045, 2015; Inoue et al., Nature 10.1038/s41586-019-1646-9. 2019). This change in RNA splicing is consistent with SF3B1's role as part of the U2 snRNP complex, which is responsible for branchsite recognition in early spliceosome assembly. During the early stages of RNA splicing, SF1 binds to the canonical branchsite with the help of the U2AF heterodimeric complex, which recognizes the canonical 3′ splice site (3′ ss) and its adjacent polypyrimidine tract. (Wahl and Luhrmann, Cell 162:456-451e. 10.1016/j.cell.2015.06.061. 2015; Wahl and Luhrmann, Cell 161:1474-e1471. 10.1016/j.cell.2015.05.050). The U2 snRNP complex is then recruited to the branchsite, where the U2 snRNA base pairs with the sequences around the branchsite, thereby replacing SF1 and U2AF. Three RNA-dependent ATPase helicases (DDX42, DDX46, and DHX15) associate with the U2 snRNP complex and perform critical roles facilitating assembly and disassembly of proteins at the branchsite during this early stage of RNA splicing. (Yang et al., Nat. Commun. 14:897. 10.1038/s41467-023-36489-x. 2023).

Cancer-associated mutations in SF3B1 have repeatedly been shown to alter the selection of branchpoints by the U2 snRNP complex and, as a consequence, result in the use of cryptic pre-mRNA 3′ ss and generation of aberrantly spliced mRNA. (Darman et al., Cell Rep. 13:1033-1045, 2015; Inoue et al., Nature 10.1038/s41586-019-1646-9. 2019; Zhang et al., Mol. Cell. 76:82-95.e87, 2019. doi: 10.1016/jmolcel.2019.07.017). Despite the consistency of this result, it is not entirely clear how mutations in SF3B1 result in mis-splicing. One prevailing hypothesis is that mutations in SF3B1 disrupt physical interactions between SF3B1 and auxiliary splicing factors required for normal branchsite recognition. For example, several studies have suggested that mutations in SF3B1 weaken the interaction of SF3B1 with a variety of candidate RNA splicing-related proteins, including SUGP1 (Zhang et al., Mol. Cell. 76:82-95.e87, 2019. doi: 10.1016/jmolcel.2019.07.017), Prp5 (Tang et al., Genes Dev. 302710-2723, 2016. doi: 10.1101/gad.291872.116), and DDX42 (Yang et al., Nat. Commun. 14:897. 10.1038/s41467-023-36489-x. 2023; Zhao et al., J. Biochem. 172:117-126, 2022. doi: 10.1093/jb/mvac049). Of these, a body of data has implicated the G-patch domain-containing protein SUGP1 (SURP and G-patch domain containing 1) in the pathogenic changes induced by mutant SF3B1. (Zhang et al., Mol. Cell. 76:82-95.e87, 2019. doi: 10.1016/jmolcel.2019.07.017; Zhang et al., Proc. Natl. Acad. Sci. USA 119: e2216712119. doi: 10.1073/pnas.221612119, 2020; Liu et al., Proc. Natl. Acad. Sci. USA 117:10305-10312, 2020. doi: 10.1073/pnas. 1922622117; Alsafadi et al., Oncogene 40:85-96 2021. doi: 10.1038/s41388-020-01507-5). SUGP1 is involved in splicing fidelity during early spliceosome assembly and physically associates the SF3b and U2AF complexes with the ATP-dependent RNA helicase DHX15. (Zhang et al., Proc. Natl. Acad. Sci. USA 119: e2216712119. doi: 10.1073/pnas.221612119, 2020; Beusch et al., Mol. Cell 83:2578-2594 e2579, 2023. doi: 10.1016/j.molcel.2023.06.003; Feng et al., bioRxiv. 2022.2011.2014.516533. doi: 10.1101/2022.11.14.516533).

The G-patch domain is a short (~45 amino acid), flexible, glycine-rich motif which binds and stabilizes the conformations of DHX helicases in a manner that increases their RNA affinities, helicase, and ATPase activities. (Studer et al., Proc. Natl. Acad. Sci. USA 117:7159-7170, 2020. doi: 10.1073/pnas. 1913880117; Bohnsack et al., Biol. Chem. 402:561-579, 2021. doi: 10.1515/hsz-2020-0338). The G-patch domain of SUGP1 activates the helicase activity of DHX15, thereby allowing it to displace SF1 from the branchsite and permitting U2 snRNA to recognize the branchsite. (Zhang et al., Mol. Cell 76:82-95 e87, 2019. doi: 10.1016/j.molcel.2019.07.017; Beusch et al., Mol. Cell 83:2578-2594 e2579, 2023. doi: 10.1016/j.molcel.2023.06.003; Feng et al., bioRxiv. 2022.2011.2014.516533. doi: 10.1101/2022.11.14.516533). Zhang et al. identified that cancer-associated mutations in SF3B1 reduce SF3B1's affinity to SUGP1, thereby reducing eviction of SF1 from the branchsite and prohibiting accessibility of the SF3b complex to the correct branchsite. (Zhang et al., Mol. Cell 76:82-95 e87, 2019. doi: 10.1016/j.molcel.2019.07.017). This impairment in SF1 removal from pre-mRNA forces the U2 snRNP complex to utilize aberrant, cryptic branchpoints characteristic of SF3B1-mutant cells. In support of this model, cancers with naturally occurring somatic mutations in SUGP1 as well as DHX15 mimic some of the pathogenic splicing changes unique to SF3B1-mutant cancer cells. (Liu et al., Proc. Natl. Acad. Sci. USA 117:10305-10312, 2020. doi: 10.1073/pnas.1922622117; Alsafadi et al., Oncogene 40:85-96 2021. doi: 10.1038/s41388-020-01507-5).

While previous studies reveal how mutations in SF3B1 may induce mis-splicing, the present disclosure identifies trans- and cis-acting proteins required for mis-splicing by mutant SF3B1. Taking advantage of mutant SF3B1-dependent mis-splicing activity, synthetic intronic sequences, which are efficiently excised in SF3B1 wild-type (WT) cells but not SF3B1-mutant cells, were developed. This approach was harnessed to drive selective expression of a fluorescent protein to perform high-throughput, unbiased positive selection screens for genes whose deletion corrects aberrant mutant SF3B1-dependent mis-splicing activity. These studies revealed the previously poorly characterized G-patch domain-containing protein GPATCH8 as selectively required for mis-splicing across diverse SF3B1-mutant models and the multitude of SF3B1 hotspot mutations. These findings are of fundamental biological importance, as it reveals that GPATCH8, bearing an eponymous G-patch domain, interacts with RNA and DHX15 and is important in branchpoint selection against suboptimal branchpoints. Moreover, deletion of GPATCH8 was tolerated in WT hematopoietic cells and corrected both molecular splicing changes as well as the impaired hematopoiesis characteristic of SF3B1-mutant human and murine cells, thus demonstrating a potential therapeutic avenue. Finally, the present disclosure demonstrates the power of synthetic intronic sequences to identify molecular bases for RNA mis-splicing in disease.

The present disclosure demonstrates that GPATCH8 is important in recognition of 3′ splice sites, such that suppression of GPATCH8 function or expression corrected key disease-causing errors in splicing that are caused by SF3B1 mutations. Furthermore, suppression of GPATCH8 expression rescued key disease phenotypes of cells bearing SF3B1 mutations, demonstrating the therapeutic promise of this approach. Suppressing or antagonizing GPATCH8 expression or function is therefore a promising way to both revert disease-causing splicing changes that are hallmarks of SF3B1-mutant cells and rescue disease phenotypes, indicating that GPATCH8 is an important therapeutic target for treating malignancies with SF3B1 mutations.

The present disclosure utilized synthetic introns uniquely responsive to cancer-associated mutations in SF3B1 to perform unbiased, positive enrichment, whole-genome CRISPR screens. The use of the synthetic intron reporter strategy identified the G-patch domain-containing protein GPATCH8 as required for approximately one-third of the several thousand aberrant RNA splicing events induced by mutant SF3B1. Further, GPATCH8 was also identified as a novel RNA splicing factor which normally serves in the quality control of branchpoint selection. The synthetic intron reporter strategy also facilitated high-throughput screens to delineate domains required for splicing activity and comparisons of the impact of distinct proteins on splicing regulation. These assays identified that splicing regulation by GPATCH8 requires its G-patch domain, conserved glycine residues within its G-patch domain, and all the N-terminus containing the coiled-coil and C2H2 zinc finger domains adjacent to the G-patch domain.

Recent studies have identified a related G-patch domain-containing protein SUGP1 as also important in branchpoint recognition. The present disclosure identifies an important role for GPATCH8 in the molecular and phenotypic sequelae of SF3B1 mutations. Several reports suggest that cancer-associated mutations in SF3B1 result in displacement of SUGP1 from the spliceosome, which, in turn, could be partially responsible for the pathophysiologic effects of the SF3B1 mutation. Along the same lines, one possible explanation for the requirement of GPATCH8 for SF3B1 mutation-dependent mis-splicing could be that mutations in SF3B1 promote physical interaction with GPATCH8.

SUGP1 has been shown now across several studies to physically link U2 snRNP to the RNA helicase DHX15 via SUGP1's G-patch domain. Further, the present disclosure identifies that GPATCH8 also physically interacts with DHX15. As described earlier, recruitment of DHX15 to U2 snRNP is critical for branchpoint recognition, as the ATPase helicase function of DHX15 displaces SF1 and may allow U2 snRNP to identify correct branchpoints. Consistent with this, the inventors identified that expression of a mutant form of SUGP1's G-patch domain which impairs DHX15 interaction phenocopies certain aberrant splicing events induced by mutant SF3B1. At the same time, reconstitution of GPATCH8-null cells with the G-patch domain-containing N-terminus of GPATCH8 alone is sufficient to recapitulate the aberrant splicing seen with mutant SF3B1. Moreover, despite the conservation of the G-patch domain across SUGP1 and GPATCH8, the inventors demonstrate that the G-patch domains of these proteins are not interchangeable. These data suggest that GPATCH8 and SUGP1 might compete for interaction with DHX15, which could explain the functional antagonism between SUGP1 and GPATCH8 in regulating SF3B1 mutation-dependent mis-splicing. In this model, loss, or mutation of SUGP1 could favor GPATCH8/DHX15 interaction, thereby sequestering DHX15 from recruitment to U2 snRNP for correct branchsite recognition.

SF3B1 mutations occur at several distinct amino acid residues, several of which are linked to unique RNA splicing events and associated with specific types of cancers. For example, there is a clear association of SF3B1 R625 mutations to melanoma, and SF3B1 K666 mutations are associated with adverse outcome in MDS. Currently, the basis for these allele-specific associations of individual SF3B1 mutations and specific cancers and cancer outcomes are unknown. Nonetheless, GPATCH8 deletion was required for mis-splicing induced by each of these most encountered mutations in SF3B1. This point was tested across cell lines from different cancer backgrounds and in primary cells from several new genetically engineered mouse models of Sf3b1 mutations (including mice with Sf3b1 K700E, K666N, and R625H mutations).

The present disclosure demonstrates that deleting GPATCH8 allowed for the correction of a multitude of SF3B1 mutant mis-splicing events in parallel. In so doing, GPATCH8 deletion partially rescued the impaired hematopoietic growth and erythroid differentiation characteristic of SF3B1 mutations in mouse and human hematopoietic precursor cells. Ineffective erythropoiesis is the hallmark of SF3B1 mutant MDS and was recapitulated by SF3B1-edited primary human HSPCs. GPATCH8 deletion corrected the erythroid differentiation defect, and this effect was most pronounced in early erythropoiesis, consistent with the role of MAP3K7 mis-splicing in regulating erythroid master transcription factor GATA127. This data thereby links several SF3B1 mutant mis-splicing events to the impaired hematopoiesis induced by SF3B1 mutations. It is important to note that GPATCH8 is not a pan-essential gene across cancer cell lines and interestingly deletion of GPATCH8 did not abrogate growth of human or mouse hematopoietic precursors in vitro. Overall, the present disclosure provides support for pharmacologic approaches to disable GPATCH8, or its interaction with DHX15, as an important therapeutic approach to correct molecular and biological defects of SF3B1 mutant cancers.

In accordance with the foregoing, in one aspect, the disclosure provides for a method of treating a subject with cancer. In some embodiments, the cancer can be characterized by a change-of-function or loss-of-function mutation in a recurrently mutated RNA splicing factor gene. In some embodiments, the method can comprise administering to a subject a therapeutically effective amount of a composition that inhibits expression of a trans-acting splicing factor and/or inhibits expression of a cis-acting splicing factor, wherein a cancer-causing error triggered by the change-of-function or loss-of-function mutation in a recurrently mutated RNA splicing factor gene can be partially or fully rescued by inhibiting expression of the trans-acting splicing factor and/or inhibiting expression of the cis-acting splicing factor. In still other embodiments, the method can comprise administering to a subject a therapeutically effective amount of a composition that inhibits function of a trans-acting splicing factor and/or inhibits function of a cis-acting splicing factor, wherein a cancer-causing error triggered by the change-of-function or loss-of-function mutation in a recurrently mutated RNA splicing factor gene can be partially or fully rescued by inhibiting function of the trans-acting splicing factor and/or inhibiting the function of the cis-acting splicing factor.

In some embodiments, the recurrently mutated RNA splicing factor gene can comprise SF3B1. In some embodiments, the recurrent change-of-function mutation in SF3B1 can result in an amino acid substitution that can comprise E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, I704F, G740E, G742D, A762V, Y765C, D781E, D781G, M784I, E802Q, M971T, M971V, or combinations thereof, with reference to the wild-type amino acid sequence set forth in SEQ ID NO:1.

In some embodiments, the cancer can be a myelodysplastic syndrome (MDS), a chronic myelomonocytic leukemia (CMML), a chronic lymphocytic leukemia (CLL), an acute myeloid leukemia (AML), a uveal melanoma, a mucosal melanoma, a skin melanoma, a breast cancer, a pancreatic cancer, an endometrial cancer, a liver cancer, a lung cancer, a mesothelioma, or other cancers with recurrent SF3B1 mutations.

In some embodiments, the composition can comprise a nucleic acid construct that can inhibit expression of a trans-acting splicing factor and/or inhibit expression of a cis-acting splicing factor by attenuating the expression of a target gene, wherein the trans-acting splicing factor and/or the cis-acting splicing factor are encoded by the target gene. In some embodiments, the nucleic acid construct can comprise any construct used for inhibiting gene expression that is well-known to one of ordinary skill in the art. In some embodiments, constructs used to inhibit gene expression include but are not limited to antisense oligonucleotides, gene knockout via CRISPR, gene knockout via CRISPRi, or RNA-targeting CRISPR.

In still other embodiments, the composition can comprise at least one protein-binding moiety that can bind to at least one protein of interest and at least one tag that can promote degradation of the protein of interest, wherein the protein of interest can comprise the trans-acting splicing factor and/or the cis-acting splicing factor. In some embodiments, the protein-binding moiety and the tag comprise proteolysis-targeting chimera (PROTAC) molecules. In other embodiments, molecular glue is used to create an interaction between the protein of interest and a second protein, wherein the interaction between the protein of interest and the second protein results in the degradation of the protein of interest. In still other embodiments, the method can comprise any well-known method used by one of ordinary skill in the art for degrading at least one protein of interest.

In still other embodiments, the composition can comprise a small molecule that can disrupt a protein-protein interaction required for the trans-acting splicing factor and/or the cis-acting splicing factor to recognize and bind to its splice site. In some embodiments, the small molecule can bind to a trans-acting splicing factor and/or a cis-acting splicing factor, which can block binding of the trans-acting splicing factor and/or the cis-acting splicing factor to at least one facilitating protein, wherein the trans-acting splicing factor and/or the cis-acting splicing factor cannot bind to its splice site without first binding to the facilitating protein. In other embodiments, the small molecule can bind to at least one facilitating protein, which can block binding of the facilitating protein to the trans-acting splicing factor and/or the cis-acting splicing factor, wherein the trans-acting splicing factor and/or the cis-acting splicing factor cannot bind to its splice site without first binding to the facilitating protein. In some embodiments, the method to inhibit protein function can comprise any method to inhibit protein function that is well-known to one of ordinary skill in the art. As used here a “facilitating protein” is any protein that can specifically bind to a trans-acting splicing factor and/or a cis-acting splicing factor and the interaction of the trans-acting splicing factor and/or a cis-acting splicing factor with the facilitating protein is necessary for the trans-acting splicing factor and/or a cis-acting splicing factor to bind to its splicing site.

In some embodiments, the trans-acting splicing factor can comprise a protein encoded by a GPATCH8 gene.

In some embodiments, the protein encoded by the GPATCH8 gene can be a G patch domain-containing protein 8 (GPATCH8) protein.

In some embodiments, the therapeutically effective amount of the composition can inhibit expression of the GPATCH8 protein by at least 25% (e.g., 25%, 30%, 35%, 40%, 45%, 50%, 55%, 60%, 65%, 70%, 75%, 80%, 85%, 90%, 95%, or 100%). In some embodiments, the GPATCH8 protein cannot bind to its 3′ recognition splice site if GPATCH8 protein expression is reduced by at least 25% (e.g., 25%, 30%, 35%, 40%, 45%, 50%, 55%, 60%, 65%, 70%, 75%, 80%, 85%, 90%, 95%, or 100%) thereby partially or fully rescuing mis-splicing caused by the mutation in a recurrently mutated RNA splicing factor gene. In still other embodiments, the therapeutically effective amount of the composition can inhibit function of the GPATCH8 protein by at least 25% (e.g., 25%, 30%, 35%, 40%, 45%, 50%, 55%, 60%, 65%, 70%, 75%, 80%, 85%, 90%, 95%, or 100%). In some embodiments, the GPATCH8 protein cannot bind to its 3′ recognition splice site if GPATCH8 protein function is reduced by at least 25% (e.g., 25%, 30%, 35%, 40%, 45%, 50%, 55%, 60%, 65%, 70%, 75%, 80%, 85%, 90%, 95%, or 100%), thereby partially or fully rescuing mis-splicing caused by the mutation in a recurrently mutated RNA splicing factor gene.

In some embodiments, the composition can further comprise a therapeutic agent.

In another aspect, the disclosure provides for an in vitro method to screen for a trans-acting splicing factor and/or a cis-acting splicing factor required for mis-splicing in a change-of-function or loss-of-function mutation in a recurrently mutated RNA splicing factor gene. In some embodiments, the method can comprising: (a) generating an expression cassette comprising a coding sequence (CDS) interrupted by at least one artificial nucleic acid intron, a promoter operatively linked to the CDS, wherein upon splicing of the artificial nucleic acid intron the CDS encodes a detectable reporter protein in wild-type cells and wherein the CDS does not encode a detectable reporter protein in cells comprising the recurrently mutated RNA splicing factor gene; (b) contacting the construct generated in (a) to a population of cells; (c) performing a positive enrichment CRISPR screen to identify at least one gene (i.e., gene of interest) whose knockout enhances expression of the detectable reporter protein in cells comprising the recurrently mutated RNA splicing factor gene; and (d) detecting expression of the detectable reporter protein, wherein expression of the detectable reporter protein in cells comprising the recurrently mutated RNA splicing factor gene is increased if the gene knockout is required for mis-splicing in recurrently mutated RNA splicing factor gene.

In some embodiments, the population of cells are monitored for modulation of the expression of a functional reporter protein, which indicates whether knockout of the gene of interest modulates the activity of the recurrently mutated RNA splicing factor. In some embodiments, the modulation is the presence or increase of functional reporter protein when a mutated RNA splicing factor is present and functionally active. In alternative embodiments, the modulation is the decrease or absence of functional reporter protein in when a mutated RNA splicing factor is present and functionally active.

In some embodiments, the expression cassette can comprise a promoter and/or appropriate enhancers operatively linked to the CDS. Upon processing of the transcript encoded, and potential splicing of the artificial nucleic acid intron, the CDS encodes or does not encode a functional detectable reporter protein. Splicing depends upon mutant splicing factor activity in the cell and, therefore, differs between cells with a genetic background comprising a change-of-function or loss-of-function mutation in a recurrently mutated RNA splicing factor gene and cells lacking such a mutation.

In some embodiments, the screen can be scaled up to assess the impact of a library of knockout genes of interest on aberrant RNA splicing due to change-of-function or loss-of-function mutation(s) in a recurrently mutated RNA splicing factor gene. The screen can be characterized as a positive screen, i.e., assessing for a positive effect in inhibiting aberrant RNA splicing. In some embodiments, indicated above, the cells are derived from a subject, e.g., from a biopsy. The screen can be implemented to assess how the suspected cancer in the subject might respond to a variety of candidate therapeutics. For example, the cells can be expanded and arranged in an array plate and individual cells or groups of cells are transformed with the expression cassette comprising the artificial intron and contacted with different potential therapeutics. In some embodiments, the detection of reporter protein is indicative of the aberrant splicing activity and, thus, is inversely proportional to the efficacy of the therapeutic contacted to the cells.

In other embodiments, the screen can be characterized as a negative screen. The expression cassette comprising the synthetic intron can be configured, as described above, to preferentially result in expression of a functional reporter protein in the absence of a mutated RNA splicing factor or in the presence of an inhibited mutated RNA splicing factor. Accordingly, detection of a functional reporter protein in the cell indicates that knockout of the gene of interest partially or fully rescues the phenotype caused by the mutated RNA splicing factor in the cell. In contrast, an absence or relative reduction in detected functional reporter protein in the cell indicates that knockout of the gene of interest has no effect on the phenotype caused by the mutated RNA splicing factor in the cell.

In other embodiments, the screening method can comprise a population of cells derived from a subject, e.g., a suspected cancer cell obtained from the subject. As above, the population of cells are contacted with an expression cassette comprising a coding sequence (CDS) interrupted by at least one artificial nucleic acid intron, as disclosed herein. The population of cells are monitored for expression of an intact protein resulting from a complete CDS, e.g., an intact reporter protein, which indicates aberrant activity of an RNA splicing factor and, thus, indicates the presence of a change-of-function or loss-of-function mutation in a recurrently mutated RNA splicing factor gene, as described above. Cells that exhibit aberrant RNA splicing, as indicated by presence of a protein encoded by the CDS, can be further subjected to a screen of candidate compounds that can inhibit aberrant RNA splicing to determine the appropriateness of the candidate compounds as a therapeutic. In other embodiments, cells that exhibit aberrant RNA splicing due to a mutant RNA splicing factor may have reduced expression of the protein encoded by the CDS and a screen is then conducted to monitor for production of the CDS. In this manner, a screen will identify trans-acting factors which correct aberrant RNA splicing activity of the mutation.

The candidate compounds can be any compounds suspected of having a potential direct or indirect effect on the transcription or splicing functionality in a cell. For example, candidate compounds can be selected from a small molecule, protein (e.g., antibody, or fragment or derivative thereof, enzyme, and the like), and nucleic acid construct to alter the genome or transcriptome of the cell, or a complex of a nucleic acid and protein.

In some embodiments, the artificial nucleic acid intron can comprise: a 5′ splice site; a canonical 3′ splice site; at least one cryptic 3′ splice site, that is within about 100 nucleotides upstream of the canonical 3′ splice site or within about 50 nucleotides downstream of the canonical 3′ splice site; a pyrimidine-rich domain that can comprise at least 6 consecutive nucleotides, wherein the sequence of the pyrimidine-rich domain is at least 60% pyrimidine nucleotides, and wherein the pyrimidine-rich domain is within at least 50 nucleotides of a cryptic 3′ splice site; or at least one branchpoint at least 15 nucleotides upstream of the canonical 3′ splice site.

In some embodiments, the intron can be at least about 50 nucleotides to about 1000 nucleotides in length.

In some embodiments, the intron can be derived from a human wildtype intron comprising intron 1 of MTERFD3, intron 4 of MYO15B, intron 10 of SYTL1, intron 11 of SYTL1, intron 4 of MAP3K7, intron 1 of ORAI2, or intron 1 of TMEM14C. In still other embodiments, the human wildtype intron from which the intron is derived can comprise: intron 1 of MTERFD3 comprising a sequence set forth in SEQ ID NO:2; intron 4 of MYO15B comprising a sequence set forth in SEQ ID NO:3; intron 10 of SYTL1 comprising a sequence set forth in SEQ ID NO:4; intron 11 of SYTL1 comprising a sequence set forth in SEQ ID NO:5; intron 4 of MAP3K7 comprising a sequence set forth in SEQ ID NO:6; intron 1 of ORAI2 comprising a sequence set forth in SEQ ID NO:7; or intron 1 of TMEM14C comprising a sequence set forth in SEQ ID NO:8.

In some embodiments, the intron can be derived from a human wildtype intron 4 of MAP3K7, and wherein the intron can further comprise one, two, three, or more of the following features: a 5′ splice site comprising a GT dinucleotide immediately followed by a consensus 5′ splice site context, optionally wherein the consensus 5′ splice site context can comprise AAG, GAG, or GTG; a canonical 3′ splice site that can comprise an AG dinucleotide immediately preceded by a C or T; at least one cryptic 3′ splice site, located at least 5 nucleotides upstream of the canonical 3′ splice site, with an AG dinucleotide and comprising a sequence that is a weaker 3′ splice site than is the canonical 3′ splice site, where splice site strength is estimated with the MaxEntScan algorithm or similar methods; a pyrimidine-rich domain that can comprise at least 15 consecutive nucleotides, wherein the sequence of the pyrimidine-rich domain can be at least 60% pyrimidine nucleotides and at least 40% thymine nucleotides, and wherein the pyrimidine-rich domain can be within at least 30 nucleotides of a cryptic 3′ splice site; or at least one branchpoint at least 20 nucleotides upstream of the canonical 3′ splice site.

In some embodiments, the intron can have a 5′ end domain with about 10 to about 150 nucleotides having at least 50% sequence identity to a sequence of the 5′-most 10 to about 150 nucleotides of the wildtype intron.

In some embodiments, the intron can have a 3′ end domain with about 50 to about 350 nucleotides having at least 50% sequence identity to a sequence of the 3′-most 50 to about 350 nucleotides of the wildtype intron.

In some embodiments, the intron can have a sequence with at least 75% sequence identity to a sequence selected from SEQ ID NOs: 9-28.

In some embodiments, the 5′ splice site can comprise a sequence comprising GTGAG, GTAAG, GTGCG, GTACG, GTGGG, GTAGG, GTGTG, GTATG, or GTATC.

In some embodiments, the canonical 3′ splice site can comprise a sequence comprising AAG, CAG, or TAG.

In some embodiments, the cryptic 3′ splice site can comprise a sequence comprising AAG, CAG, GAG, TAG, ATG, CTG, GTG, or TTG.

In some embodiments, the intron can comprise a plurality of cryptic 3′ splice sites within about 100 nucleotides upstream of the canonical 3′ splice site or within about 100 nucleotides downstream of the canonical 3′ splice site, and wherein each of the plurality of the cryptic 3′ splice sites can comprise a sequence independently selected from AAG, CAG, GAG, TAG, ATG, CTG, GTG, or TTG.

In some embodiments, the pyrimidine-rich domain can be characterized by one, two, three, or all of the following: wherein the pyrimidine-rich domain can comprise at least 15 consecutive nucleotides; wherein the pyrimidine-rich domain can have a sequence with at least 60% pyrimidine nucleotides and is at least 40% thymine nucleotides; wherein the pyrimidine-rich domain can be within at least 30 nucleotides of a cryptic 3′ splice site; or wherein the pyrimidine-rich domain can have a sequence with at least 50% sequence identity to any 20 nucleotides selected from the sequence set forth as SEQ ID NO: 29.

In some embodiments, the branchpoint can be at least 20 nucleotides upstream of the canonical 3′ splice site. In some embodiments, the branchpoint nucleotide can be an adenine.

In some embodiments, the branchpoint and surrounding sequence context can have a sequence identity of at least 60% to the sequence tactaAca, where the uppercase A is the branchpoint nucleotide.

In some embodiments, the intron can be configured to be spliced differently in a cancer cell comprising a change-of-function or loss-of-function mutation in a recurrently mutated RNA splicing factor gene relative to the splicing pattern of the intron in a cell lacking a change-of-function or loss-of-function mutation in a recurrently mutated RNA splicing factor gene.

In some embodiments, the RNA splicing factor gene can be SF3B1. In still other embodiments, the recurrent change-of-function mutation in SF3B1 can result in an amino acid substitution comprising E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, 1704F, G740E, G742D, A762V, Y765C, D781E, D781G, M784I, E802Q, M971T, M971V, or combinations thereof, with reference to the wild-type amino acid sequence set forth in SEQ ID NO: 1.

In some embodiments, the artificial nucleic acid intron can further comprise a first exon domain and a second exon domain, wherein the intron is disposed between the first exon domain and the second exon domain. In other embodiments, the combination of the first exon domain and the second exon domain without the intron can encode part of or all of a protein of interest.

In some embodiments, the nucleic acid intron construct can comprise an expression cassette comprising the first exon domain, the intron, the second exon domain, and a promoter sequence operatively linked thereto.

In some embodiments, detecting expression of the detectable reporter protein can comprise quantifying the amount of the reporter protein.

In some embodiments, the reporter protein can comprise a fluorescent or luminescent protein.

Additional embodiments and descriptions of the claimed methods can be found in the Appendix.

### Additional Definitions

The term “promoter” refers to a regulatory nucleotide sequence that can activate transcription (expression) of a gene. As indicated, a promoter is typically located upstream of a gene, but can be located at other regions proximal to the gene, or even within the gene. The promoter typically contains binding sites for RNA polymerase and one or more transcription factors, which participate in the assembly of the transcriptional complex. As used herein, the term “operatively linked” indicates that the promoter and the gene region (e.g., including coding and noncoding, or intron, sequence) are configured and positioned relative to each other a manner such that the promoter can activate transcription of the encoding nucleic acid by the transcriptional machinery of the cell. The promoter can be constitutive or inducible. Constitutive promoters can be determined based on the character of the target cell and the particular transcription factors available in the cytosol. A person of ordinary skill in the art can select an appropriate promoter based on the intended use, as various promoters are known and commonly used in the art. In some embodiments, the nucleic acid intron construct comprises an expression cassette comprising the first exon domain, the intron, the second exon domain, and a promoter sequence operatively linked thereto.

The expression cassette can be incorporated into a vector, such as a plasmid or viral vector, configured for delivery into a cell. Accordingly, in some embodiments, the disclosure provides a vector comprising the artificial nucleic acid intron construct described above. The vector can be any construct that facilitates the delivery of the nucleic acid to the target cell and/or expression of the nucleic acid within the cell. The vectors can be viral vectors, circular nucleic acid constructs (e.g., plasmids), or nanoparticles. Various viral vectors are known in the art and are encompassed by the present disclosure. See. e.g., Machida, C. A. (ed.), Viral Vectors for Gene Therapy: Methods and Protocols, Humana Press, Totowa, New Jersey (2003); Muzyczka, N., (ed.), Current Topics in Microbiology and Immunology: Viral Expression Vectors, Springer-Verlag, Berlin, Germany (2012), each incorporated herein by reference in its entirety. In some embodiments, the viral vector is an adeno-associated virus (AAV) vector, an adenovirus vector, a herpes simplex virus vector, a retrovirus vector, a lentivirus vector, an alphavirus vector, a flavivirus vector, a rhabdovirus vector, a measles virus vector, a Newcastle disease virus vector, a Coxsackievirus vector, or a poxvirus vector. An exemplary embodiment of an AAV vector includes the AAV2/5 serotype.

As used herein, “expression vector” refers to a DNA construct containing a nucleic acid molecule that is operatively-linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host cell, the vector may replicate and function independently of the host genome, or may, in some instances, integrate into the genome itself. In the present specification, “plasmid,” “expression plasmid,” “virus” and “vector” can be used interchangeably.

Unless specifically defined herein, all terms used herein have the same meaning as they would to one skilled in the art of the present invention. Practitioners are particularly directed to Sambrook J., et al. (eds.), Molecular Cloning: A Laboratory Manual, 3rd ed., Cold Spring Harbor Press, Plainsview, New York (2001); Ausubel, F. M., et al. (eds.), Current Protocols in Molecular Biology, John Wiley & Sons, New York (2010); and Coligan, J. E., et al. (eds.), Current Protocols in Immunology, John Wiley & Sons, New York (2010) for definitions and terms of art.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

As used herein, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type II, and type III) based on the sequence and structure of the Cas nucleases. The crRNA-guided surveillance complexes in types I and III need multiple Cas subunits. The type II system, the most studied, comprises at least three components: an RNA-guided Cas9 nuclease, a crRNA, and a trans-acting crRNA (tracrRNA). The tracrRNA comprises a duplex-forming region. A crRNA and a tracrRNA form a duplex that is capable of interacting with a Cas9 nuclease and guiding the Cas9/crRNA: tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a transgene with homologous flanking sequences can be introduced at the site of DSB via homology-directed repair. The crRNA and tracrRNA can be engineered into a single guide RNA (sgRNA or gRNA) (see, e.g., Jinek et al., Science 337:816-821, 2012). Further, the region of the guide RNA complementary to the target site can be altered or programmed to target a desired sequence (Xie et al., PLOS One 9: e100448, 2014; U.S. Pat. Appl. Pub. No. US 2014/0068797, U.S. Pat. Appl. Pub. No. US 2014/0186843; U.S. Pat. No. 8,697,359, and PCT Publication No. WO 2015/071474; each of which is incorporated by reference in its entirety). In certain embodiments, a gene knockout comprises an insertion, a deletion, a mutation, or a combination thereof, made using a CRISPR/Cas nuclease system. As used herein, a meganuclease, also referred to as a homing endonuclease, refers to an endodeoxyribonuclease characterized by a large recognition site (doublestranded DNA sequences of about 12 to about 40 base pairs). Meganucleases can be divided into five families based on sequence and structure motifs: LAGLID ADG, GIY-YIG, HNH, His-Cys box and PD-(D/E) XK. Exemplary meganucleases include I-Scel, I-Ceul, PI-PspI, PLSce, LScelV, I-CsmI, I-PanI, I-Scell, I-Ppol, I-Scein, I-Crel, I-TevI, I-TevII and I-TevIII, whose recognition sequences are known (see, e.g., U.S. Pat. Nos. 5,420,032 and 6,833,252; Belfort et al., Nucl. Acids Res. 25:3379-3388, 1997; Dujon et al., Gene 82:115-118, 1989; Perler et al., Nucl. Acids Res. 22:1125-1127, 1994; Jasin, Trends Genet. 12:224-228, 1996; Gimble et al., J. Mol. Biol. 263:163-180, 1996; Argast et al., J. Mol. Biol. 280:345-353, 1998, each of which is incorporated herein by reference in its entirety).

As indicated above, the CDS generated by splicing the artificial intron can be a protein that provides a detectable signal. The selective expression of such a reporter protein in a cancer cell can be leveraged to guide more specific and targeted surgical techniques. Accordingly, in another aspect, the disclosure provides a method of enhancing surgical resection of a tumor from a subject. In this aspect, the tumor is characterized by a change-of-function or loss-of-function mutation in a recurrently mutated RNA splicing factor gene. The method comprises administering to the subject an effective amount of a therapeutic composition comprising an expression cassette comprising a coding sequence (CDS) encoding a detectable marker, wherein the CDS is interrupted by at least one artificial nucleic acid intron as described above, and wherein the expression cassette further comprises a promoter operatively linked to the CDS.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

The terms “subject,” “individual,” and “patient” are used interchangeably herein to refer to a mammal being assessed for treatment and/or being treated. In certain embodiments, the mammal is a human. The terms “subject,” “individual,” and “patient” encompass, without limitation, individuals having cancer. While subjects may be human, the term also encompasses other mammals, particularly those mammals useful as laboratory models for human disease, e.g., mouse, rat, dog, non-human primate, and the like.

The term “treating” and grammatical variants thereof may refer to any indicia of success in the treatment or amelioration or prevention of a disease or condition (e.g., a cancer, infectious disease, or autoimmune disease), including any objective or subjective parameter such as abatement; remission; diminishing of symptoms or making the disease condition more tolerable to the patient; slowing in the rate of degeneration or decline; or making the final point of degeneration less debilitating.

The treatment or amelioration of symptoms can be based on objective or subjective parameters, including the results of an examination by a physician. Accordingly, the term “treating” includes the administration of the compounds or agents of the present disclosure to prevent or delay, to alleviate, to improve clinical outcomes, to decrease occurrence of symptoms, to improve quality of life, to lengthen disease-free status, to stabilize, to prolong survival, to arrest or inhibit development of the symptoms or conditions associated with a disease or condition (e.g., a cancer), or any combination thereof. The term “therapeutic effect” refers to the reduction, elimination, or prevention of the disease or condition, symptoms of the disease or condition, or side effects of the disease or condition in the subject.

As used herein, the term “polypeptide” or “protein” refers to a polymer in which the monomers are amino acid residues that are joined together through amide bonds. When the amino acids are alpha-amino acids, either the L-optical isomer or the D-optical isomer can be used, the L-isomers being preferred. The term “polypeptide” or “protein” as used herein encompasses any amino acid sequence and includes modified sequences such as glycoproteins. The term “polypeptide” is specifically intended to cover naturally occurring proteins, as well as those that are recombinantly or synthetically produced.

One of skill will recognize that individual substitutions, deletions or additions to a peptide, polypeptide, or protein sequence which alters, adds, or deletes a single amino acid or a percentage of amino acids in the sequence is a “conservatively modified variant” where the alteration results in the substitution of an amino acid with a chemically similar amino acid. Conservative amino acid substitution tables providing functionally similar amino acids are well known to one of ordinary skill in the art. The following six groups are examples of amino acids that are considered to be conservative substitutions for one another:
(1) Alanine (A), Serine(S), Threonine (T),
(2) Aspartic acid (D), Glutamic acid (E),
(3) Asparagine (N), Glutamine (Q),
(4) Arginine (R), Lysine (K),
(5) Isoleucine (I), Leucine (L), Methionine (M), Valine (V), and
(6) Phenylalanine (F), Tyrosine (Y), Tryptophan (W).

As used herein, the term “nucleic acid” refers to a polymer of nucleotide monomer units or “residues.” The nucleotide monomer subunits, or residues, of the nucleic acids each contain a nitrogenous base (i.e., nucleobase) a five-carbon sugar, and a phosphate group. The identity of each residue is typically indicated herein with reference to the identity of the nucleobase (or nitrogenous base) structure of each residue. Canonical nucleobases include adenine (A), guanine (G), thymine (T), uracil (U) (in RNA instead of thymine (T) residues) and cytosine (C). However, the nucleic acids of the present disclosure can include any modified nucleobase, nucleobase analogs, and/or non-canonical nucleobase, as are well-known in the art. Modifications to the nucleic acid monomers, or residues, encompass any chemical change in the structure of the nucleic acid monomer, or residue, that results in a noncanonical subunit structure. Such chemical changes can result from, for example, epigenetic modifications (such as to genomic DNA or RNA), or damage resulting from radiation, chemical, or other means. Illustrative and nonlimiting examples of noncanonical subunits, which can result from a modification, include uracil (for DNA), 5-methylcytosine, 5-hydroxymethylcytosine, 5-formethylcytosine, 5-carboxycytosine β-glucosyl-5-hydroxy-methylcytosine, 8-oxoguanine, 2-amino-adenosine, 2-amino-deoxyadenosine, 2-thiothymidine, pyrrolo-pyrimidine, 2-thiocytidine, or an abasic lesion. An abasic lesion is a location along the deoxyribose backbone but lacking a base. Known analogs of natural nucleotides hybridize to nucleic acids in a manner similar to naturally occurring nucleotides, such as peptide nucleic acids (PNAs) and phosphorothioate DNA.

Reference to sequence identity addresses the degree of similarity of two polymeric sequences, such as nucleic acid or protein sequences. Determination of sequence identity can be readily accomplished by persons of ordinary skill in the art using accepted algorithms and/or techniques. Sequence identity is typically determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. Various software driven algorithms are readily available, such as BLAST N or BLAST P to perform such comparisons.

The following description of the preferred embodiments of the present disclosure is not intended to limit the invention to these preferred embodiments, but rather to enable any person skilled in the art to make and use this invention.

### Example 1

### Cell Lines

K562 (CCL-243), MCF10A (CRL-10317), 293T (CRL-3216), Panc05.04 (CRL-2557), cells were obtained from the American Type Culture Collection (ATCC). Isogenic K562 and MCF10A cells with or without defined SF3B1 mutations were generated by Horizon Discovery using adenoviral-mediated homologous recombination and previously described. Uveal melanoma cell lines, MEL202 and MEL270, were obtained from B. Bastian and previously described. MEL270 cells ectopically expressing SF3B1WT or SF3B1K700E were generated using a doxycycline-inducible expression system as previously described and doxycycline (1 μg/mL) was added every 3 days to the media for induction. K562 cells were grown in Iscove's modified Dulbecco's media (IMDM) with 10% Fetal Bovine Serum (FBS) (Gibco). MCF10A cells were grown in DMEM/F12 supplemented with 5% horse serum (Gibco), 20 ng/ml EGF (Millipore/Sigma), 10 μg/mL insulin (Millipore/Sigma), 0.5 μg/mL hydrocortisone (MilliporeSigma) and 0.1 μg/mL cholera toxin (Millipore/Sigma). MEL270 and MEL202 cells were grown in RPMI1640 with 10% FBS (Gibco). MEL202 cells were additionally supplemented with 1% GlutaMAX™ (Gibco). Panc05.04 were grown in RPMI1640 with 20% FBS and 20 U/mL human recombinant insulin (Sigma). 293T cells were grown in DMEM supplemented with 10% FBS, 4.5 g/L glucose and 1% GlutaMAX™ (Gibco). All cell lines were grown at 37° C. and 5% atmospheric CO₂ in the presence of 100 μg/mL penicillin and 100 mg/mL streptomycin.

### Example 2

### Mice

All animal procedures were conducted in accordance with the Guidelines for the Care and Use of Laboratory Animals and approved by the Institutional Animal Care and Use Committees (IACUC) at Memorial Sloan Kettering Cancer Center (protocols 13-04-003 and 18-05-008). All mice were housed at Memorial Sloan Kettering Cancer Center with 12 h light/dark cycles and controlled temperature (20-22° C.) and humidity (40-70%). 6-8-week-old female C57BL/6 CD45.1⁺ mice were purchased from the Jackson Laboratory (stock no. 002014).

The Sf3b1 K700E conditional knockin mouse was previously described. Sf3b1 K666N and Sf3b1 R625H conditional knockin mice were generated by Ingenious Targeting Laboratory (Ronkonkoma, NY) using a targeted homologous recombination approach. Briefly, C57BL/6 FLP embryonic stem (ES) cells were electroporated with linearized targeting constructs encoding either inverted exon 12-14 (with K666N mutation) or inverted exon 12-17 (with R625H mutation) and the neomycin selection cassette. After selection with G418 antibiotic, surviving clones were expanded for PCR analysis to identify recombinant ES clones. Correctly targeted ES cells were injected into BALB/c blastocysts. The resulting chimeric animals were bred to C57BL/6 wildtype mice to generate Germline Neo Deleted mice. Final germline-transmitted mice were confirmed for the deletion of the Neomycin cassette and the presence of Sf3b1 mutant allele by genotyping tail DNA from mice with black coat color and sequencing the PCR product.

Sf3b1 mutant mice were bred to Mx1-cre mice (The Jackson Laboratory, Strain #: 003556) to generate Mx1-cre Sf3b1mut/WT mice. Mouse genotypes from tail biopsies were determined using real time PCR with specific probes designed for each allele (Transnetyx, Cordova, TN). Expression of Cre recombinase was induced with three polyinosine-polycytosine injections (pIpC; 12 mg/kg; GE Healthcare) every other day to achieve recombination in hematopoietic tissue.

### Example 3

### Gene Editing of Human Cord Blood HSPCs

Human CD34⁺ cells isolated from umbilical cord blood were edited at the SF3B1 locus via CRISPR/Cas9 and adeno-associated virus (AAV6) template delivery system described in Sarchi et. al. Edited cells integrated a BFP fluorescent reporter in SF3B1 intron 14 and either p.2098 A>G K700E mutation or wild-type K700K sequence. Simultaneous gene editing was performed using SF3B1 and either GPATCH8 or AAVS1 non-targeting control sgRNAs with the Cas9 complex at a 1:2.5 molar ratio for each sgRNA. The sequences of sgRNAs used are as follows: SF3B1 5′-UGGAUGAGCAGCAGAAAGUU-3′ (SEQ ID NO:65), AAVS1 NO: 66), GPATCH8 5′-GGGCCACUAGGGACAGGAU-3′ (SEQ ID 5′-ATGCTGAAGATGCTACCGAA-3′ (SEQ ID NO:67).

Editing efficiency was assessed 48 hours post-editing via flow cytometry for BFP and PCR amplification of edited loci, followed by Sanger sequencing and Inference of CRISPR Edits (ICE) analysis (Synthego).

### Example 4

### Human HSPC Erythroid Differentiation

Erythroid differentiation was performed as described in Clough et al. (Blood 139:2038-2049, 2022. doi: 10.1182/blood.2021012652). Briefly, primary edited CD34⁺ HSPCs were cultured for 6 days in IMDM (ThermoFisher)+15% FBS (Sigma)+1% BSA (ThermoFisher) with 100 U/mL penicillin/streptomycin (Fisher), 2 mM L-glutamine (ThermoFisher), 500 μg/mL holo-Transferrin (Sigma), 10 μg/mL Insulin (Cell Sciences), 100 ng/mL SCF, 5 ng/mL IL-3, and 6 U/mL erythropoietin (Procrit). After 6 days, IL-3 was removed and SCF reduced to 50 ng/ml, and the culture continued for another 14 days. Cells were initially plated at a density of 1-2×10⁵/mL and maintained <106/mL.

### Example 5

### Vector Cloning, Lentiviral Production, and Cell Transduction

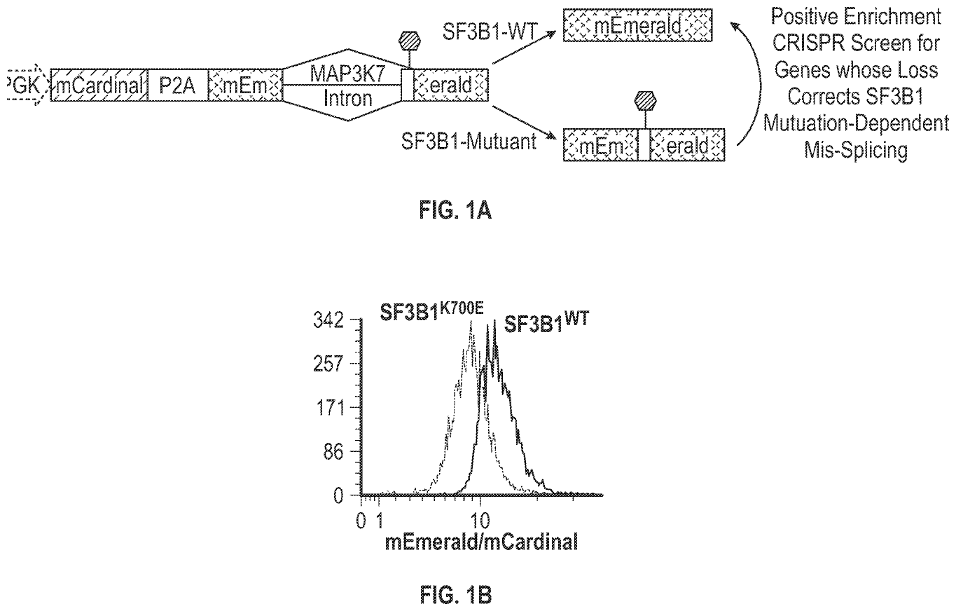

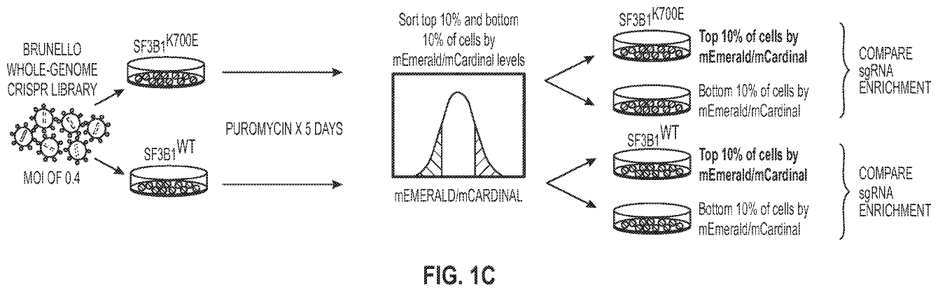

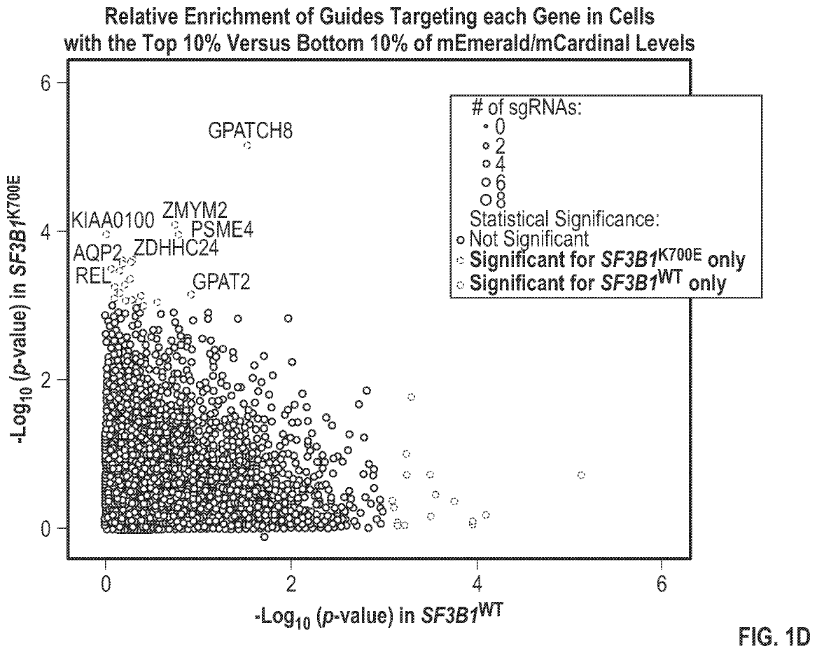

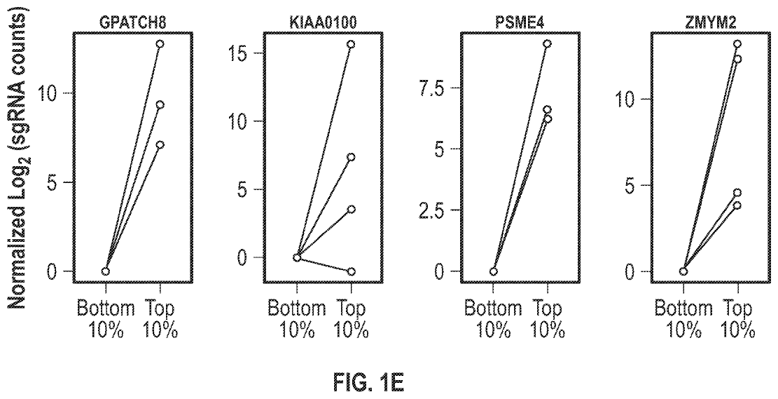

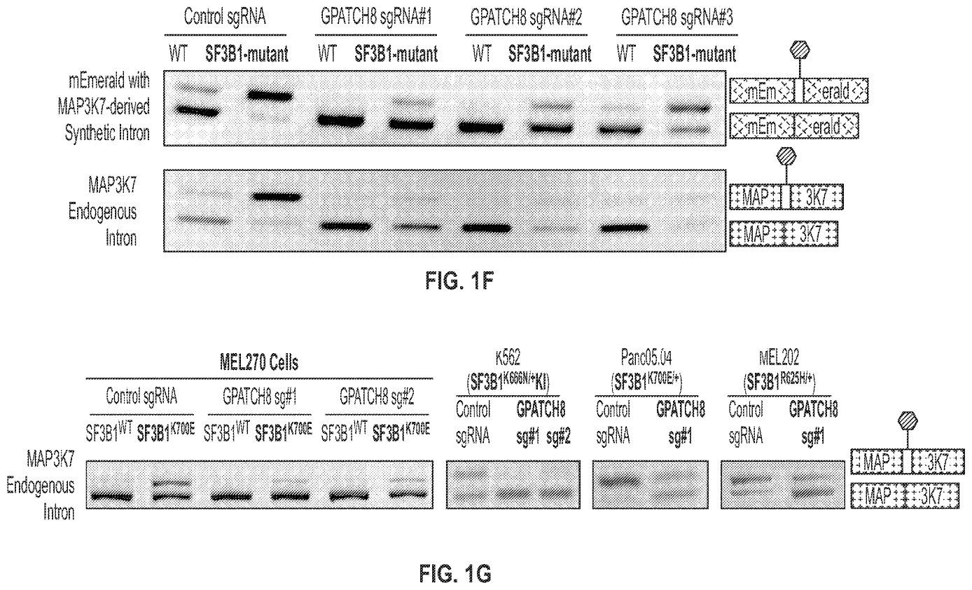

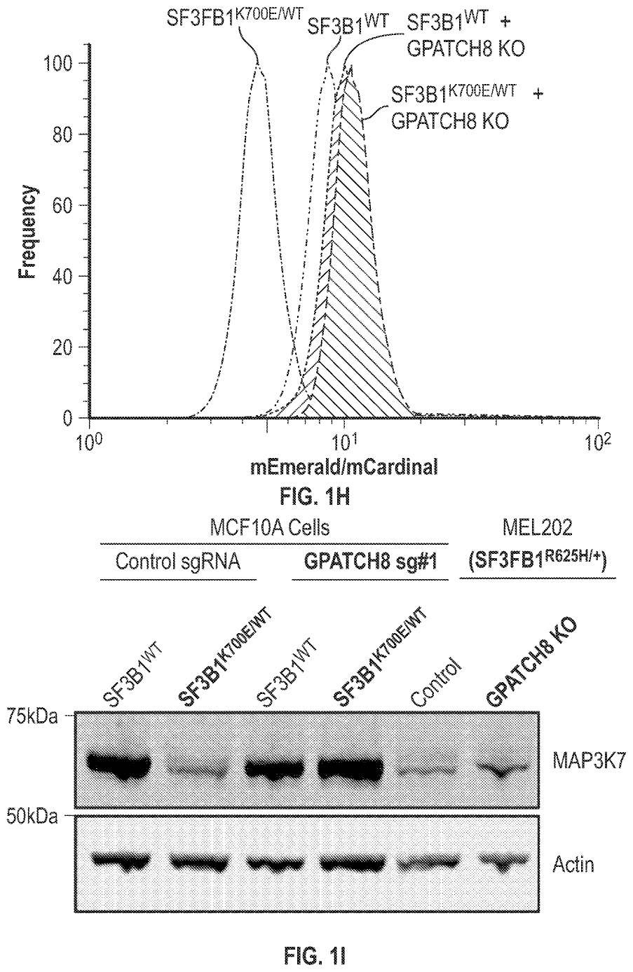

To generate hPGK-mCardinal-P2A-mEmerald-synMAP3K7i250 vector, the mCardinal-P2A and P2A-mEmerald-synMAP3K7i250 fragments were amplified from mCardinal-pBiCMV-mEmerald-synMAP3K7i4-250 using primers described in the specification. Fragments with correct sizes were purified on agarose gel and cloned into hPGK-PuroR-P2A-HSV-TK to replace the PuroR-P2A-HSV-TK sequence by Gibson assembly (NEBuilder® HiFi, New England Biolabs). The orientation of the fragments was as illustrated in FIG. 1A.

Individual sgRNAs were cloned into the lentiviral vector LentiGuide-Puro. shRNAs were cloned into a modified version of SGEP (constitutive lentiviral miR-E) where green fluorescent protein (GFP) was replaced by Crimson fluorescent protein.

TRIBE plasmids used either RBP-ADAR or ADAR-RBP fusions with a downstream p2A-GFP to allow for fluorescence-based sorting of transfected cells. GPATCH8 was synthesized (GenScript) upstream of the Drosophila ADAR catalytic domain containing both hyperediting (E488Q) and auto editing blocking mutations (S458 synonymized) (47) (Addgene #154787). SUGP1 was synthesized downstream of the Drosophila ADAR catalytic domain containing both hyperediting (E488Q) and auto editing blocking mutations (S458 synonymized). For control plasmids either synonymized MS2 coat protein dimer (MCP) fused to the ADAR catalytic domain (stdMCP-ADAR) was used as a GPATCH8-ADAR control (47) (Addgene #154787) or in a flipped orientation (ADAR-stdMCP) for the ADAR-SUGP1 control. All TRIBE plasmids were subcloned from Addgene #154787 and sequence verified by GenScript.

For GPATCH8 rescue experiments, codon optimized GPATCH8, SUGP1 and their variants were synthetized and cloned into pGenLenti expression vector (GenScript) with a downstream Linker-NLS-3×FLAG-T2A-BFP. All pGenLenti variant plasmids were cloned, and sequence verified by GenScript.

All lentiviruses were produced in 293T cells transfected with 4:2:3 ratios of expression plasmid:pVSVG:psPAX2 and 3:1 ratio of Polyethylenimine (PEI):DNA. Viral-containing supernatant was collected 48 and 72 h after transfection, filtered through 0.22 μm filter, supplemented with 4 μg/mL or the cationic polymer Polybrene, and used for cell transduction.

### Example 6

3×-FLAG-Tag Knockin into Endogenous GPATCH8

Isogenic SF3B1WT and SF3B1K700E/WT K562 cells were further edited by Synthego (Redwood City, CA) using CRISPR-Cas9 to insert 3×-FLAG-tag into endogenous GPATCH8 N-terminus. Single positive clones with homozygous insertion were selected. The sgRNA sequence was GAGGAGUGAAGGCGGCAAAA (SEQ ID NO: 68), the donor DNA sequence was GGCGACGCCCGTGTTTACCTGAA AGTCTCGGTCTTCGTTGAAGCGGGAGAAGCGGTCCGCTCCGGAGCTGCCCTT GTCGTCGTCGTCCTTGTAGTCGATGTCGTGGTCCTTGTAGTCACCGTCGTGGT CCTTGTAGTCCATGTTGCCGCCTTCACTCCTCTCAGGACGACGCTCTCCGGTTC GCTCCTTCCCTCCTGC (SEQ ID NO:69), the PCR and sequencing primers were GGGAACCGGGGAGGGGTGGGTG (forward; SEQ ID NO:70) and AGAGGGCTGGGAGCCGGAGATGA (reverse; SEQ ID NO:71).

### Example 7

### Whole-Genome CRISPR-Cas9 Screening

Lentiviruses carrying human CRISPR Brunello lentiviral pooled sgRNA library were produced in HEK-293T cells. Virus titer was determined by testing the percentage of puromycin-resistant cells after transducing MCF10A cells. A titer resulting in 40% of transduced cells (MOI 0.4) was selected for the screening. MCF10A WT or SF3B1 K700E mutant cells stably expressing Cas9 and hPGK-mCardinal-P2A-mEmerald-synMAP3K7i250 were transduced with lentivirus expressing human CRISPR Brunello sgRNA library. Puromycin selection (2 μg/mL) was performed 2 days after transduction in growth media for 5 days. The surviving cells were sorted by FACS to select the top 10% and bottom 10% cells by the ratio of mEmerald and mCardinal intensities. Cell pellets were lysed, and genomic DNA was extracted (E.Z.N.A.® Tissue DNA kit, Omega Biotek) and quantified by Qubit (Thermo Scientific). A quantity of gDNA covering 500× representation of gRNAs was PCR-amplified using TaKaRa™ Ex Taq™ DNA Polymerase (Takara) to add Illumina™ adapters and multiplexing barcodes. Amplicons were quantified by Qubit and Bioanalyzer (Agilent) and sequenced on an Illumina™ HiSeq™ 2500. Sequencing reads were aligned to the Brunello sgRNA library, and counts were obtained for each sgRNA. The probe level analysis was performed using the standard edgeR workflow with the glmLRT option for the model fitting/statistical test.

### Example 8

### RT-PCR and Quantitative RT-PCR

Total RNA was isolated using RNeasy® Mini kit (Qiagen) and reverse-transcribed (RT) to cDNA using Verso™ cDNA synthesis kit (Thermo Scientific). PCRs were performed using GoTaq® Green Master Mix (Promega) and amplicons were analyzed using 3% agarose gel electrophoresis. Quantitative RT-PCR (qRT-PCR) was performed using PowerUp™ SYBR™ Green PCR Master Mix and analyzed on QuantStudio™ 6 Flex Cycler (Applied Biosystems). Relative gene expression levels were calculated using the comparative threshold cycle method.

### Example 9

### Flow Cytometry

mCardinal and mEmerald visualization was assessed at least 4 days after cell transduction with hPGK-mCardinal-P2A-mEmerald-synMAP3K7i250 vectors, using 7AAD and GFP filters respectively. All cells were resuspended in DPBS containing 1% BSA and 0.4 ng/mL DAPI (Invitrogen) prior to analysis to gate on viable (DAPI negative) cells. For GPATCH8 rescue experiments, mCardinal/mEmerald analysis was gated on BFP positive cells and DAPI was not used. All flow cytometry analyses were performed on BD LSRFortessa™ cytometer and data were analyzed on FlowJo™ (BD Biosciences).

### Example 10

### Western Blotting

Protein samples were extracted from cultured cells with RIPA buffer (Thermo Fisher Scientific) and quantified by BCA assay. Protein fractionated on NuPAGE 4%-12% Bis-Tris gels (Life Technologies) was transferred onto nitrocellulose membranes. All primary antibodies were used with 1:1000 dilution in TBST (Tris-buffered saline with 1% Tween® 20) containing 5% BSA and 0.02% sodium azide. Goat anti-Rabbit IgG Secondary Antibody was used at 1:10000 dilution in TBST containing 5% skim milk. The blot was then visualized by fluorescence on Odessey® Imaging System (LI-COR). Precision Plus Protein™ Kaleidoscope™ Prestained Protein Standards (Bio-Rad) was used for protein size markers.

### Example 11

### RNA-Sequencing Library Preparation and Sequencing

For cell line RNA sequencing (RNA-seq), RNA was extracted from MCF10A or K562 cells using the Qiagen™ RNeasy® extraction kit, according to the manufacturer's instructions. A minimum of 500 ng of high-quality RNA (as determined by Agilent Bioanalyzer) per replicate was used as input for library preparation. Poly(A)-selected, strand-specific (dUTP method) Illumina libraries were prepared by the Integrated Genomics Operation (IGO) at Memorial Sloan Kettering with a modified TruSeq™ protocol and sequenced on the Illumina™ HiSeq™ 2000 to obtain ~60-80M paired-end 100 bp reads per sample.

### Example 12

### RNA-Seq Data Analysis

A genome annotation (UCSC hg19/GRCh37) was created by combining the University of California Santa Cruz knownGene (Karolchik et al., Nucl. Acids Res. 42: D764-770, 2014. doi: 10.1093/nar/gkt1168), Ensembl 71 (Flicek et al., Nucl. Acids Res. 41: D48-55, 2013. doi: 10.1093/nar/gks1236), and MISO v2.0 (Katz et al., Nat. Methods 7:1009-1015, 2010. doi: 10.1038/nmeth.1528) annotations. Transcriptome mapping was accomplished using RSEM v1.2.454 with the argument “-v 2” which indicates Bowtie invocation. This mapping strategy also generates gene expression values, in units of transcripts per million (TPM). All RSEM-generated gene expression estimates were normalized by applying the trimmed mean of M values (TMM) method. Unaligned reads were mapped to the genome with TopHat v2.0.8b, and to a custom annotation which includes all gene-level pairwise combinations of annotated 5′ and 3′ splice sites. The combined RSEM/Bowtie and TopHat alignments were inputted to MISO v2.0 to quantify isoform expression levels. Differential isoform expression between sample groups was ascertained using the two-sided t-test. Differentially spliced events were defined as containing at least 20 isoform-specific reads, a minimum absolute difference of 10% in isoform expression, and a p-value<0.05.

### Example 13

### Splice Site Sequence Analyses

Differentially spliced cassette exon events following GPATCH or SUGP1 deletion were identified. 10-mer and 54-mer sequences which contain the 5′ and 3′ splice site dinucleotide motifs, respectively, were generated from treatment-responsive splicing events. The inventors previously identified branchpoint nucleotide coordinates through a large-scale analysis of intron lariat-derived reads from thousands of RNA-seq datasets. These positions were used to define 9-mer branchpoint sequences. The extracted sequences were used to generate sequence logo plots, visualizations of the associated position weight matrices, via the seqLogo package. (seqLogo: Sequence logos for DNA sequence alignments; R package version 1.66.0). The enrichment of purines/pyrimidines in skipped cassette exons (relative to included) was measured within and around the exonic regions using the GenomicRanges (Lawrence et al., PLoS Comput. Biol. 9: e1003118, 2013. doi: 10.1371/journal.pcbi. 1003118). The 95% confidence interval was estimated with bootstrapping (1000 resampling iterations). The analyses were performed within the R Programming environment with tools from Bioconductor (Huber et al., Nat. Methods 12:115-121, 2015. doi: 10.1038/nmeth.3252), visualized using the tidyverse (Wickham et al., J. Open Source Software 10.21105/joss.01686) packages.

### Example 14

### Colony-Forming Assays

Four weeks after pIpC treatment of Mx1-cre Sf3b1 mutant or WT mice, bone marrow cells were collected and c-Kit⁺ hematopoietic precursors were magnetically separated using murine CD117 MicroBeads according to the manufacturer's instructions (Miltenyi Biotech) then cultured overnight in IMDM with 20% FBS supplemented with mSCF (20 ng/ml), mFLT3L (10 ng/ml) and mTPO (20 ng/ml). For the next 2 day, cells were subjected to spinfections at 2,300 rpm, for 90 min at 32° C. with lentiviral supernatant expressing shRNAs against Gpatch8 or Renilla non-targeting control in the presence of 8 μg/mL Polybrene (Sigma Aldrich). 48 h after transduction, viable shRNA-expressing cells (DAPI-Crimson⁺) were FACS-sorted, seeded into cytokine-supplemented methylcellulose medium (MethoCult™ M3434; STEMCELL Technologies) and incubated at 37° C. with 5% CO₂ and ≥95% humidity. Colonies were scored after 10 days of culture, manually using an inverted microscope.

### Example 15

### GPATCH8 and SUGP1 TRIBE-Seq

293T were transfected with GPATCH8-ADAR, ADAR-SUGP1, or respective MCP/ADAR non-targeting control, fusion expression plasmids using a 3:1 ratio of Polyethylenimine (PEI):DNA. 24 hours after transfections cells were suspended in sorting buffer (DPBS, 1% BSA supplemented with DAPI) and passed through a 35 μm strainer mesh. FACS was performed by selecting for green fluorescent protein (GFP) positive DAPI negative single cells. One million GFP positive cells were sorted on a BD FACSymphony™ S6 or BD FACSAria™ III Cell Sorter, and RNA was isolated using RNeasy® Plus Mini Kit. A minimum of 500 ng of high-quality RNA (as determined by Agilent Bioanalyzer) per replicate was used as input for library preparation. Ribo-depleted Illumina™ libraries were prepared by the Integrated Genomics Operation (IGO) at Memorial Sloan Kettering with a modified TruSeq™ protocol and sequenced on the Illumina™ HiSeq™ 2000 to obtain ~60-80 M paired-end 100 bp reads per sample.

### Example 16

### Analysis of TRIBE-Seq Data

TRIBE-seq data was analyzed following the previously published pipeline. In brief, reads from the sequencing libraries are trimmed (Trimmomatic) and aligned (STAR) to the hg19 human reference genome. The aligned BAM file is converted to a SAM file and the nucleotide frequency at each position in the transcriptome is recorded from aligned reads and placed into a SQL table. For each nucleotide in the transcriptome, the TRIBE RNA nucleotide frequency is compared with the wild-type mRNA library (WT RNA) nucleotide frequency to identify RNA editing sites. For a legitimate edit site, the frequency of A is greater than 80% and the frequency of G is 0.5% in the WT RNA and the frequency of G is greater than 0 in the TRIBE RNA (using the reverse complement if an annotated gene is in the reverse strand). Modifying the nucleotide frequency of G in WT RNA from 0 to 0.5% allows very low-level sequence heterogeneity in WT RNA and does not disrupt the identification of legitimate editing sites in deeply sequenced libraries. An editing threshold of at least 5% was used in each replicate. The TRIBE edit sites are required to be present in both replicates and any sites that overlap with MCP/ADAR control edit sites with at least 1% editing are removed. After background subtraction, BED files containing all edit sites present in both experimental replicates are analyzed using RNAmod.

### Example 17

### CRISPR Tiling Screen

sgRNA sequences targeting the coding regions of human GPATCH8 and human SUGP1 were designed using Benchling's CRISPR design tool and cloned into LentiGuide-mTagBFP vector as two separate libraries, each containing 10% of non-targeting and safe harbor sgRNAs. Lentiviral particles containing sgRNA libraries were produced in 293T cells and lentiviral supernatant was pre-titrated in K562 cells to obtain 10-20% infection rate (monitored by flow cytometry for BFP expression). K562 WT or SF3B1 K700E mutant cells stably expressing Cas9 and hPGK-mCardinal-P2A-mEmerald-synMAP3K7i250 were transduced with the pre-titrated sgRNA library viruses. 48 h post-transduction, top 10% and bottom 10% mEmerald/mCardinal expressing cells, gated on BFP⁺ cells, were sorted. The number of collected cells for each population covered at least 1000×the number of constructs in each library. Genomic DNA was extracted and representation of sgRNAs was analyzed similarly to whole-genome CRISPR-Cas9 Screening described above.

### Example 18

### Alphafold2 and ColabFold Modeling of Protein-Protein Interactions

Alphafold2 and ColabFold (Mirdita et al., Nat. Methods 19:679-682, 2022. doi: 10.1038/s41592-022-01488-1) was utilized to model the interactions between proteins of interest using default settings. To facilitate computational modeling of the proteins, ColabFold models were generated using the structured parts of the proteins and unstructured regions were excluded. For the SUGP1 DHX15 structure, the recently published crystal structure (PDB:8EJM) (Zhang et al. Proc. Natl. Acad. Sci. USA 119: e2216712119, 2022. doi: 10.1073/pnas.2216712119) was used. This structure included amino acids 113-795 of DHX15 and G-patch amino acids 543-614 of SUGP1. For the DHX15 and GPATCH8 models amino acids 68-795 of DHX15 and amino acids 1-188 of GPATCH8 (encompassing the coiled coil, G-patch and zinc finger domains of the protein) were used.

### Example 19

### Immunoprecipitation (IP) for Mass Spectrometry

Proteins were extracted from whole cells using IP lysis buffer (Pierce) and quantified using Qubit™ Protein Assay (Invitrogen). 2 mg of proteins were incubated overnight at 4° C. with Protein A agarose beads (Millipore) conjugated to FLAG antibody (clone M2, Sigma) resuspended in IP lysis buffer (Pierce). Beads were subsequently washed three times using IP lysis buffer and three times using 10 mM Tris-HCl/150 mM NaCl pH 7.5.

### Example 20

### Protein Digestion for Proteomic Analyses

The beads were resuspended in 40 μL of 2M Urea, 50 mM ammonium bicarbonate pH 8.5 and treated with DL-dithiothreitol (1 mM final concentration) for 30 minutes at 37° C. with shaking (1100 rpm) on a ThermoMixer® (Thermo Fisher). Free cysteine residues were alkylated with 2-iodoacetamide (3.67 mM final concentration) for 45 minutes at 25° C. at 1100 rpm in the dark. LysC (750 ng) was added, followed by incubation for 1 hour at 37° C. at 1150 rpm. Finally, trypsin (750 ng) was added, followed by incubation for 16 hours at 37° C. at 1150 rpm.

After incubation, the digest was acidified to pH<3 with the addition of 50% of trifluoroacetic acid (TFA), and the peptides were desalted on 3-plug C18 (3M Empore™ high performance extraction disks) stage tips. Briefly, the stage tips were conditioned by sequential addition of: i) 100 μL 100% acetonitrile (ACN), ii) 100 μL 70% ACN/0.1% TFA, iii) 100 μL 0.1% formic acid (FA), iv) 100 μL 0.1% FA. Following conditioning, the acidified peptide digest was loaded onto the stage tip. The stationary phase was washed once with 100 μL of 0.1% FA. Finally, samples were eluted using 50 μL of 70% ACN/0.1% FA twice. Eluted peptides were dried under vacuum followed by reconstitution in 12 μL of 0.1% FA, sonication and transfer to an autosampler vial. Peptide yield was quantified by NanoDrop™ (Thermo Fisher).

### Example 21

### Mass Spectrometry Analyses

Peptides were separated on a 50 cm column composed of C18 stationary phase (Thermo Fisher ES903) using a gradient from 0.5% to 25% buffer B over 100 minutes, to 50% in 15 minutes, to 90% in 5 minutes (buffer A: 0.1% FA in HPLC grade water; buffer B: 99.9% ACN, 0.1% FA) with a flow rate of 300 nL/min using a nanoACQUITY UPLC® system (Waters). MS data were acquired on an Eclipse™ mass spectrometer (Thermo Fisher Scientific) using a data-independent acquisition (DIA) method. The method consisted of one MS1 scan, AGC target Standard, maximum injection time of 50 msec, scan range of 380-985 m/z and a resolution of 120K. Fragment ions were analyzed in 60 DIA windows at a resolution of 15K.

### Example 22

### DIA Data Analysis

Raw data files were processed using Spectronaut® version 17.4 (Biognosys) and searched with the PULSAR search engine with a Homo sapiens UniProt protein database downloaded on 2022 Sep. 23 (226,953 entries). Cysteine carbamidomethylation was specified as fixed modifications, while methionine oxidation, acetylation of the protein N-terminus and deamidation (NQ) were set as variable modification. A maximum of two trypsin missed cleavages were permitted. The searches used a reversed sequence decoy strategy to control peptide false discovery rate (FDR) and 1% FDR was set as threshold for identification. Unpaired t-test was used to calculate p-value in differential analysis, volcano plot was generated based on log 2 fold change and q-value (multiple testing corrected p-value). A q-value of ≤0.05 was considered the statistically significant cut-off.

### Example 23

RNA-seq and TRIBE-seq data have been deposited to the Gene Expression Omnibus (GEO) functional genomics data repository under accession ID GSE242094. The mass spectrometry proteomics data have been deposited to the ProteomeXchange Consortium via the PRIDE partner repository with the dataset identifier PXD044973.

### Example 24

### Synthetic Introns Reveal Trans-Acting Factors Required for Mutant SF3B1-Dependent Mis-Splicing

The aberrant RNA splicing of SF3B1 mutant cells was harnessed to develop synthetic introns that were efficiently spliced in cancer cells bearing SF3B1 mutations but unspliced in otherwise isogenic WT cells. This approach allows for quantitative assessment of protein production when the synthetic intron is used to regulate the expression of a fluorescent protein.

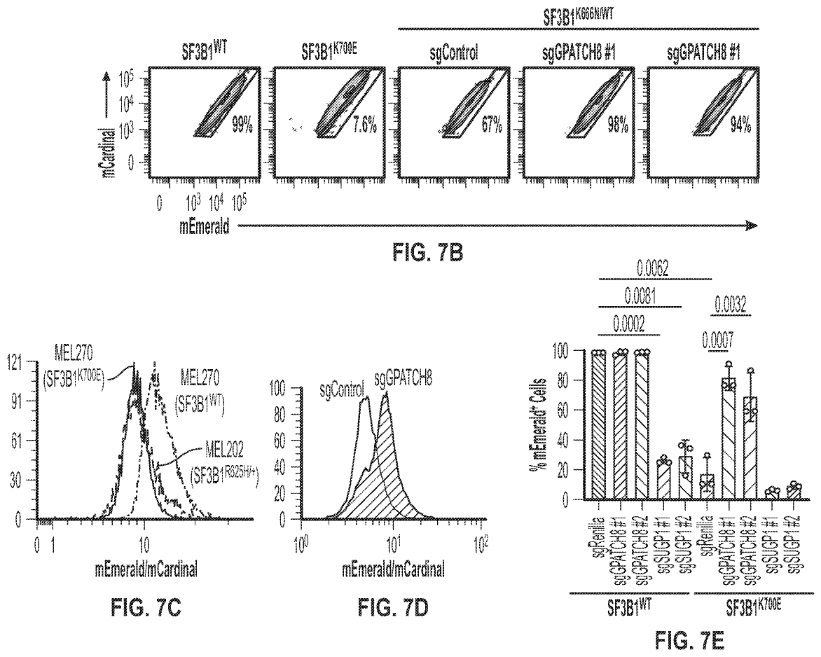

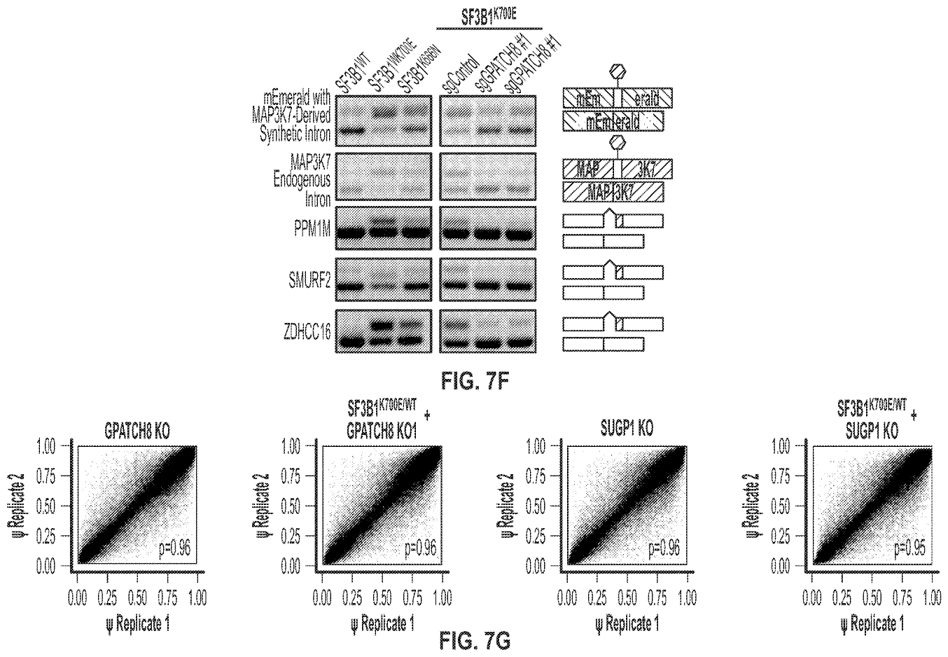

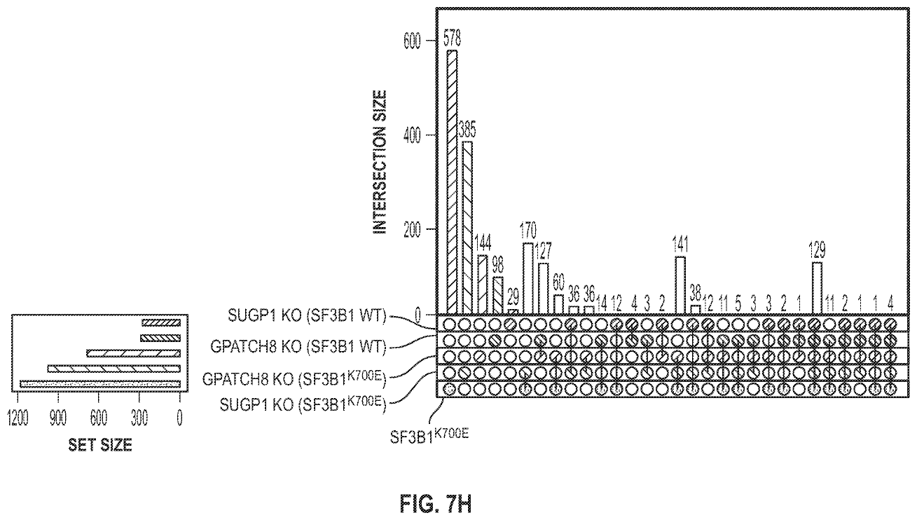

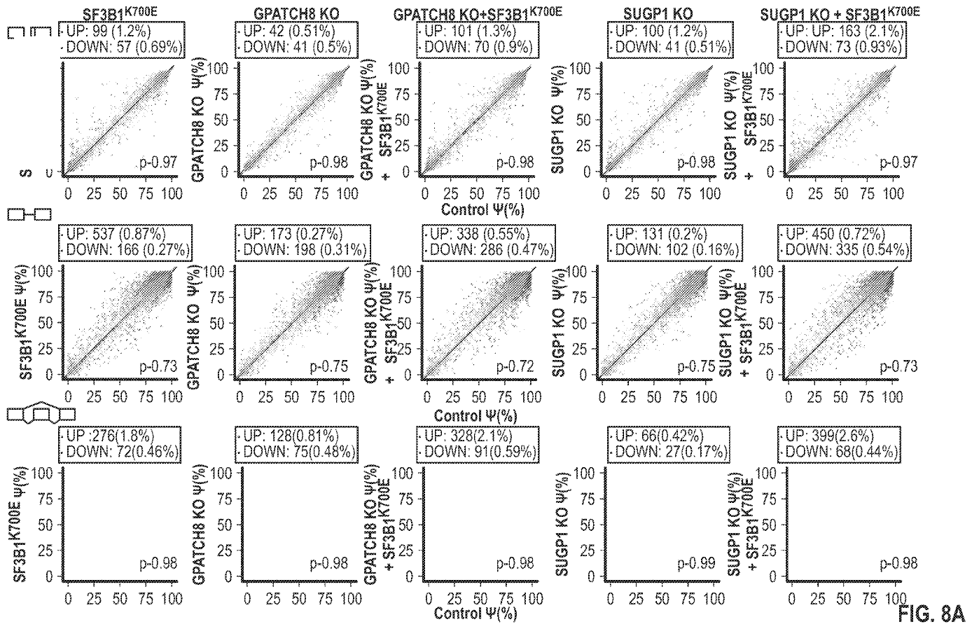

It was initially hypothesized that synthetic intron-regulated expression of fluorescent protein production could serve as a powerful tool to discover trans-acting proteins required for mutant SF3B1 aberrant splicing activity. To accomplish this, a synthetic intron based on endogenous mis-splicing of the kinase gene MAP3K7 was utilized. MAP3K7 has consistently been shown to be mis-spliced across SF3B1 hotspot mutations, cancer cell backgrounds, and human and mouse cells. SF3B1 mutation-dependent mis-splicing of MAP3K7 results in use of an aberrant intron-proximal 3′ ss and open-reading frame disruption in a manner that triggers nonsense-mediated mRNA decay (NMD) and reduces MAP3K7 protein production. While the endogenous MAP3K7 mis-spliced intron is 1407 nt, the synthetic version is 250 nt in length and consists of only the first 100 nt and last 150 nt of the mis-spliced intron. When introduced into the coding sequence of mEmerald within a vector that allows for constitutive expression of mCardinal, SF3B1-mutant cells stably expressing this construct produce ~1.9-fold less fluorescent protein (as read out by the mEmerald:mCardinal ratio via flow cytometry) than isogenic SF3B1-WT cells (FIGS. 1A-1B, FIG. 7A).

This particular synthetic intron construct was utilized for positive enrichment whole-genome CRISPR screens to identify proteins whose loss resulted in correction of SF3B1 mutation-dependent mis-splicing of the MAP3K7 synthetic intron, as read out by single guide RNAs (sgRNAs) enriched in the top 10% of mEmerald:mCardinal expressing SF3B1-mutant cells (FIG. 1C). This screen was performed in MCF10A breast cancer cells with or without endogenous knockin of the SF3B1K700E mutation and stable expression of the MAP3K7 synthetic intron construct in biological duplicate. Following transduction and stable selection of the Brunello whole-genome CRISPR sgRNA library in these cells, the top and bottom 10% of mEmerald:mCardinal expressing cells were sorted and compared sgRNAs enriched in these populations across SF3B1-mutant and WT cells (FIG. 1C).

The positive enrichment screen revealed a number of genes where >3 sgRNAs were statistically enriched in the mEmerald:mCardinal high population in SF3B1-mutant cells relative to WT, including GPATCH8, KIAA0100, PSME4, and ZMYM2 (FIGS. 1D-1E). Given the association of G-patch domain-containing proteins with RNA metabolism and recent studies linking SUGP1, another G-patch domain-containing protein, to mutant SF3B1 splicing, the potential role of GPATCH8 in SF3B1 mutation-dependent mis-splicing was evaluated next. In validation studies using a set of individual sgRNAs against GPATCH8, knockout (KO) of GPATCH8 corrected MAP3K7 synthetic intron mis-splicing as revealed by RT-PCR of the reporter transcript, RNA sequencing (RNA-seq) of the endogenous transcript, and a ~2.2-fold increase in the mEmerald:mCardinal fluorescent protein reporter (FIGS. 1F-1H, FIGS. 7A-7D).

Beyond impacting the synthetic intronic sequence, GPATCH8 KO also corrected mis-splicing of the endogenous MAP3K7 transcript (FIGS. 1F-1G). The correction of mis-splicing of endogenous MAP3K7 upon GPATCH8 KO was consistent across cellular backgrounds, including breast cancer (MCF10A), AML (K562), pancreatic cancer (Panc05.04), and uveal melanoma (MEL270 and MEL202) cell lines with either genetically engineered or naturally occurring SF3B1 mutations (FIGS. 1F-1H, FIGS. 7C-7D). Moreover, GPATCH8 KO also corrected mis-splicing across all recurrent SF3B1 missense mutations, including K700E, K666N, and R625H mutations (FIGS. 1F-1H, FIGS. 7C-7D). As noted earlier, SF3B1 mutation-dependent mis-splicing of MAP3K7 resulted in reduced MAP3K7 protein expression due to NMD. Consistent with the correction of MAP3K7 mis-splicing by GPATCH8 KO, stable GPATCH8 deletion also rescued MAP3K7 protein levels in a variety of SF3B1-mutant cancer cell lines (FIG. 1I).

### Example 25

### Antagonistic Effects of SUGP1 Versus GPATCH8 Deletion on SF3B1 Mutation-Induced Aberrant RNA Splicing

The above studies clearly link GPATCH8 to mis-splicing of MAP3K7 by mutant SF3B1. The impact of GPATCH8 deletion on RNA splicing genome-wide was determined next. RNA-seq analyses of both AML (K562) and breast cancer (MCF10A) cells were performed with or without knockin of the SF3B1K700E mutation and with or without GPATCH8 deletion.

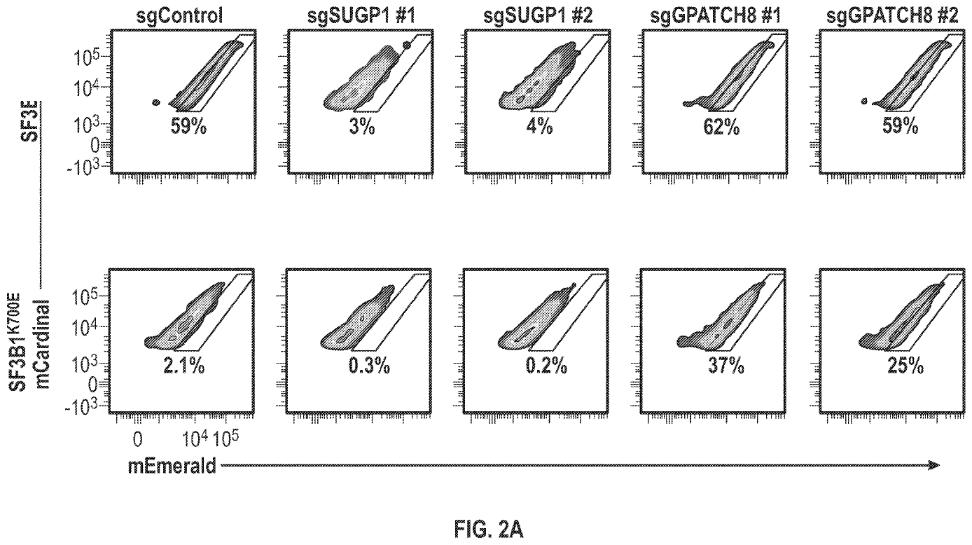

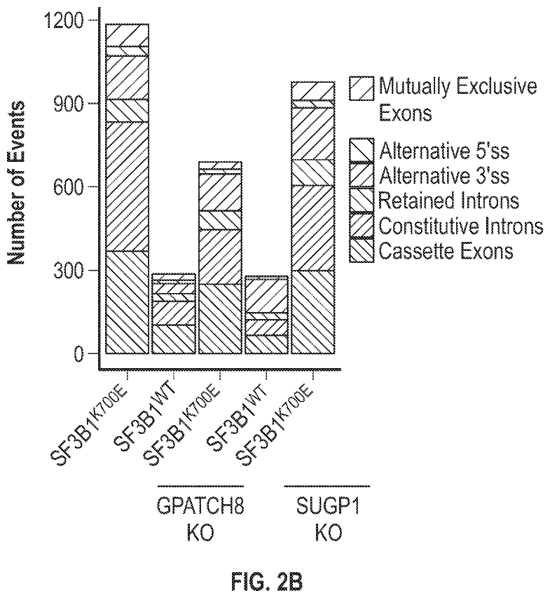

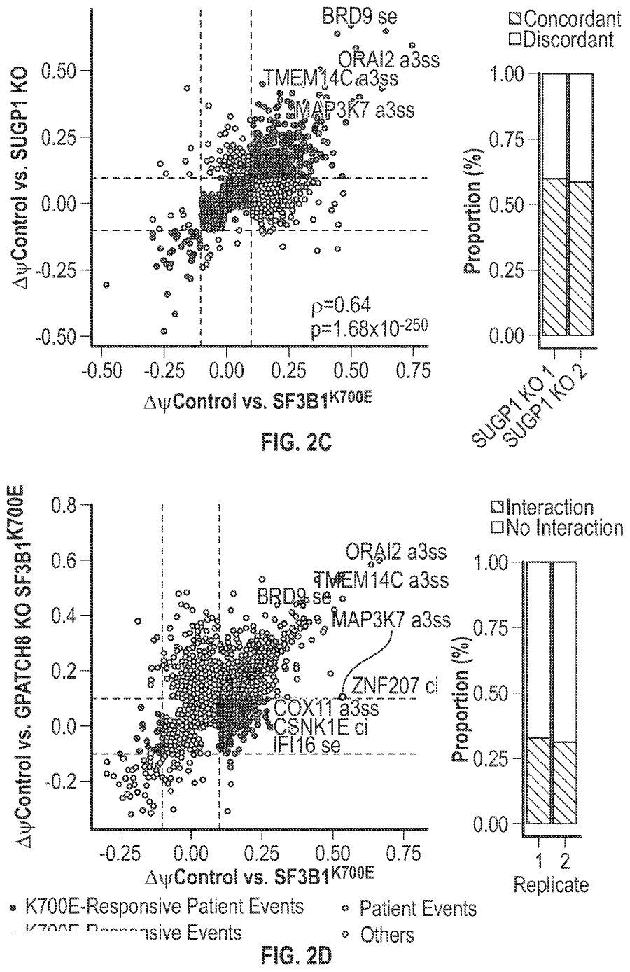

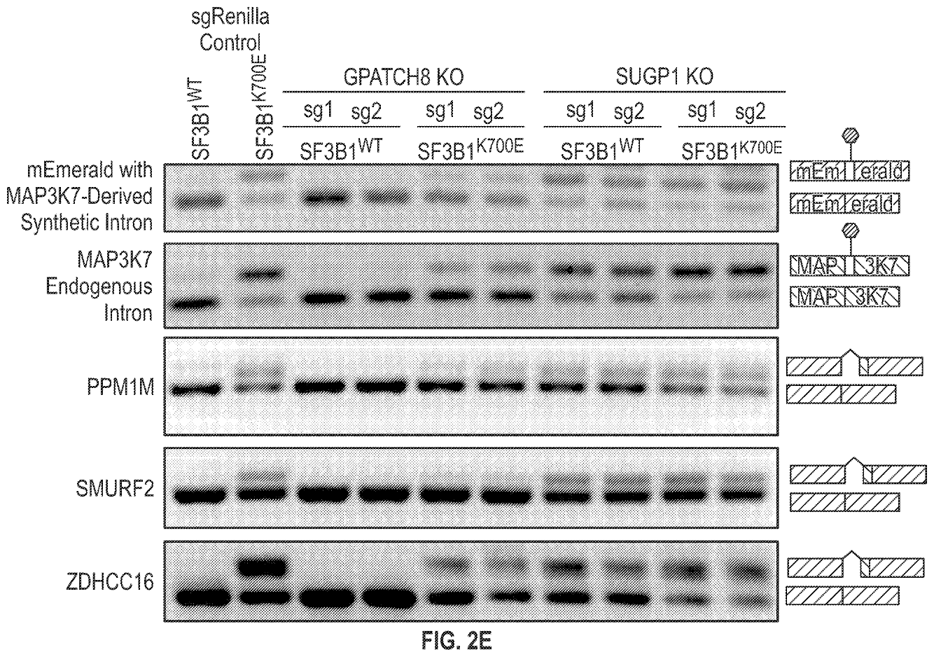

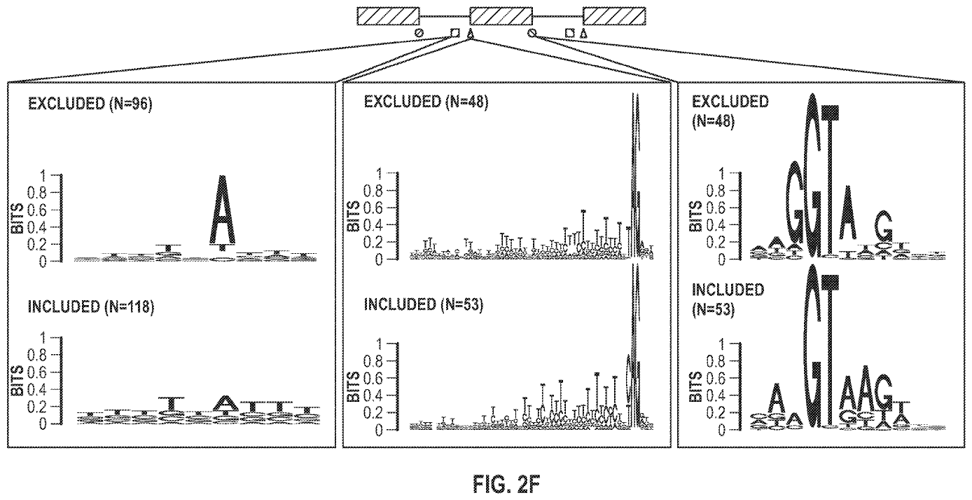

As noted earlier, SUGP1 is one of >20 human G-patch domain-containing proteins, and several recent studies have suggested that disruption of SUGP1 recapitulates some of the RNA splicing defects caused by mutant SF3B1. Interestingly, using the MAP3K7 synthetic intron bi-chromatic reporter, SUGP1 deletion phenocopied SF3B1 mutations with both alterations resulting in a very similar reduction in mEmerald:mCardinal fluorescent protein compared to parental WT cells. In contrast, GPATCH8 deletion massively increased mEmerald:mCardinal fluorescent protein in SF3B1-mutant cells (as seen earlier) (FIG. 2A, FIG. 7E). RNA-seq studies were utilized to globally compare the impact of SUGP1 deletion to GPATCH8 deletion or SF3B1 mutation.

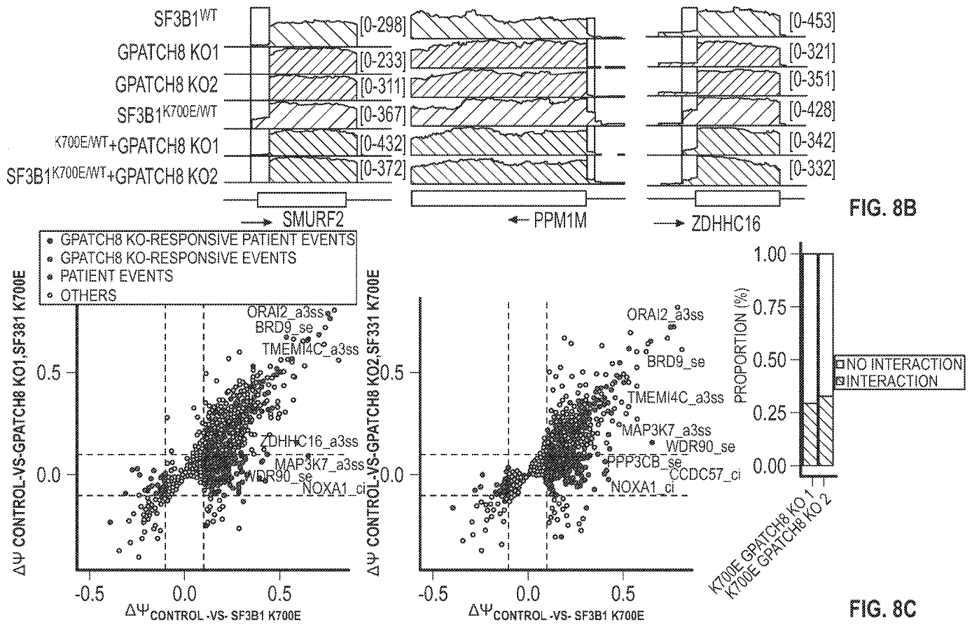

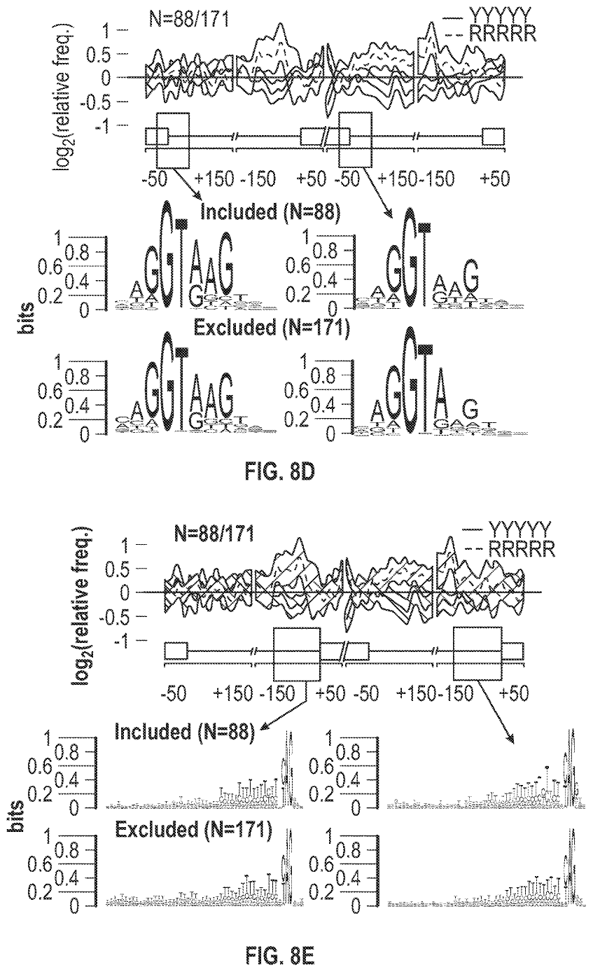

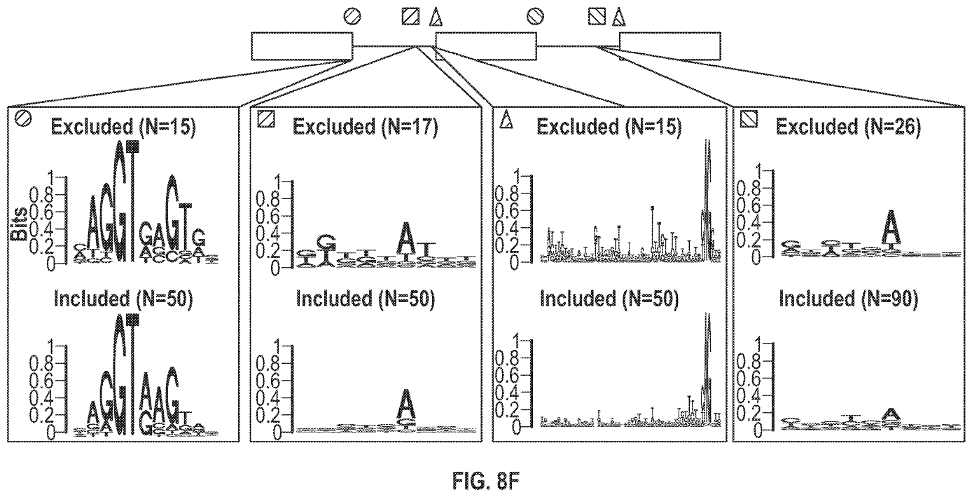

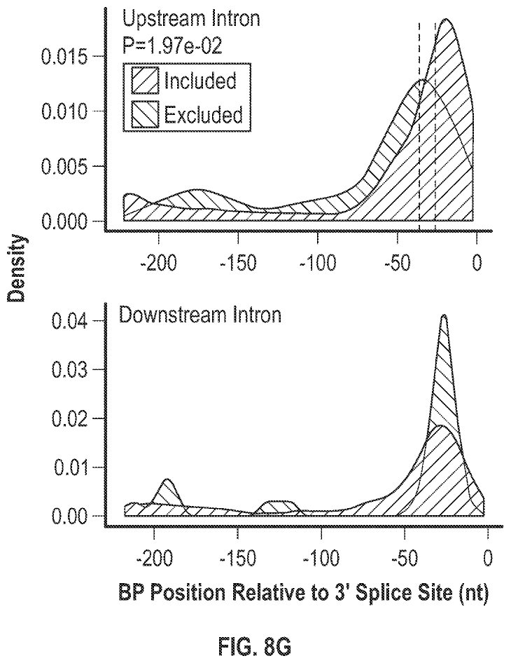

After confirming reproducibility of each RNA-seq replicate, it was found that deletion of either GPATCH8 or SUGP1 alone resulted in global alterations of RNA splicing compared to parental cells (FIGS. 7E-7H). Importantly, however, the changes in RNA splicing induced by deletion of either G-patch domain protein were less than the magnitude of changes seen with the SF3B1K700E mutation (FIG. 2B and FIG. 7H). The impact of each RNA splicing factor perturbation on categories of alternative RNA splicing events and the directionality of each type of splicing event was evaluated next. Cassette exon splicing, followed by intron retention, and then alternative 3′ss selection were the most impacted types of splicing events seen with SF3B1 mutation or deletion of either GPATCH8 or SUGP1 (FIG. 2B and FIG. 8A). Consistent with prior studies, mutation of SF3B1 or deletion of SUGP1 promoted usage of a more intron-proximal alternative 3′ ss. Overall, ~60% of RNA splicing events showed concordant treatment responses—differential or unperturbed—between the SF3B1K700E mutation and the SUGP1 deletion, inclusive of splicing events observed in SF3B1-mutant AML (28) (FIG. 2C). The conformity between responses represents a reproducible, statistically significant positive correlation between these two RNA splicing perturbants (Pearson's correlation=0.64, p<2.2×10⁻¹⁶). In contrast, deletion of GPATCH8 corrects ~30% of SF3B1K700E mutation-dependent aberrant RNA splicing events (FIG. 2D). Thus, GPATCH8 deletion opposes a portion of the RNA splicing defects created by mutant SF3B1, while SUGP1 deletion phenocopies a portion of SF3B1-mutant aberrations. This contrasting activity of GPATCH8 and SUGP1 on mutant SF3B1 was very apparent by RNA-seq and RT-PCR of splicing events within MAP3K7, PPM1M, SMURF2, and ZDHHC16, as a few examples (FIG. 2E). Importantly, the impact of GPATCH8 deletion on RNA splicing alterations caused by mutant SF3B1 was seen across different cancer cell lines (K562 and MCF10A cells) as well as distinct SF3B1 hotspot mutations (FIG. 7F and FIGS. 8B-8E).

Given that cassette exon splicing events were the most altered type of splicing change with SF3B1 mutation, GPATCH8 deletion, or SUGP1 deletion, splicing signals in the introns flanking cassette exons were evaluated next to gain insight into how GPATCH8 and SUGP1 regulate splicing. Cassette exons included upon GPATCH8 KO are associated with weaker splicing signals in the upstream intron: shorter polypyrimidine tracts and degenerate branchpoint (BP) motifs. While the spatial distribution of branchpoints differ based on response to GPATCH8 KO, with excluded exons linked to upstream introns containing more intron-proximal branchpoints, majority of the branchpoints are restricted to a small region within the 3′ intron (FIGS. 2F-2G). Each of these findings suggest that GPATCH8 may normally serve a role in suppressing the usage of weaker, suboptimal BPs which may otherwise act as competitive signals to influence alternative splicing. The upstream intron may be particularly important, as it represents the first site of splicing regulation in cassette exons in the context of co-transcriptional splicing. Interestingly, excluded exons also are associated with downstream introns containing 5′ splice sites which appear divergent from the canonical GTRAGT motif: increased preference for A at the +3 position and decreased preference for A at the +4 position, relative to the intron-exon boundary (FIG. 2F). The differences in the 5′ and 3′ splice sites flanking GPATCH8 KO-responsive cassette exons suggest that GPATCH8 could also function in regulating exon definition. Importantly, the sequence features characteristic of GPATCH8 KO-responsive cassette exons are recapitulated in experiments with both MCF10A and K562 cells (FIGS. 8D-8E).

A similar series of analyses of the splicing signals surrounding differentially utilized cassette exons upon SUGP1 deletion (FIGS. 8F-8G) were performed. Interestingly, the SUGP1 KO-responsive cassette exons are also typified by splice site features: the upstream introns of exons skipped upon SUGP1 deletion have virtually indiscernible polypyrimidine tracts, as well as a higher proportion of BPs which better conform to the YUNAY consensus motif (29) and are located further from the 3′ ss. It was hypothesized that these splice site variations are linked to concomitant differences in splicing efficiency. As such, SUGP1 could have a role in regulating 3′ ss definition possibly through a BP-mediated mechanism consistent with a recent study suggesting a role for SUGP1 in the quality control function of DHX15 at repressing suboptimal introns.

### Example 26

### GPATCH8 and SUGP1 Bind Intronic Sequences in Pre-mRNA and the G-Patch Domain of Each Protein is Required for Splicing Regulation

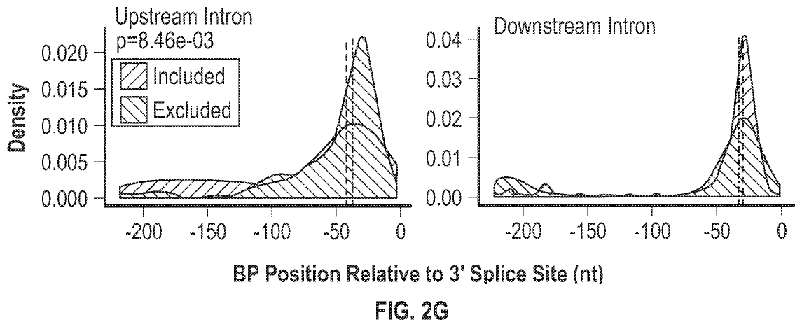

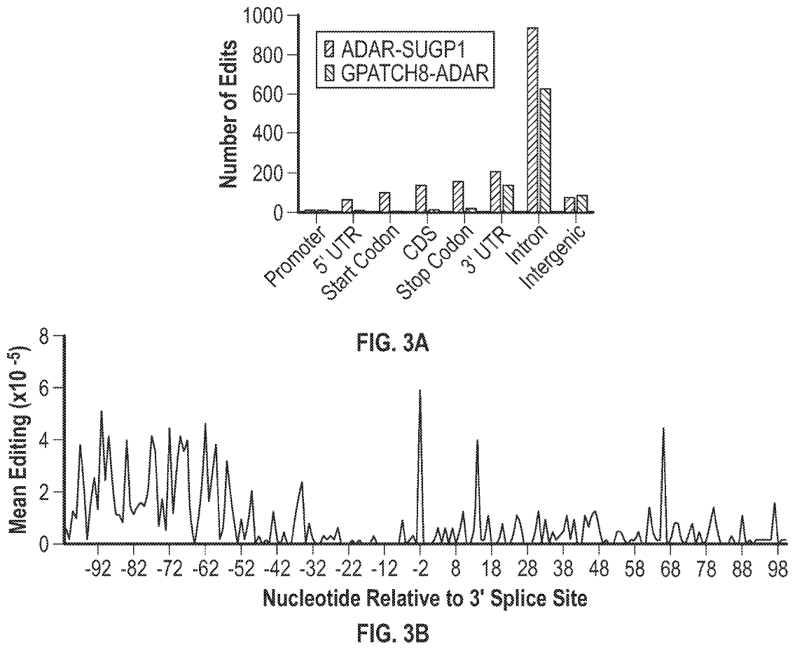

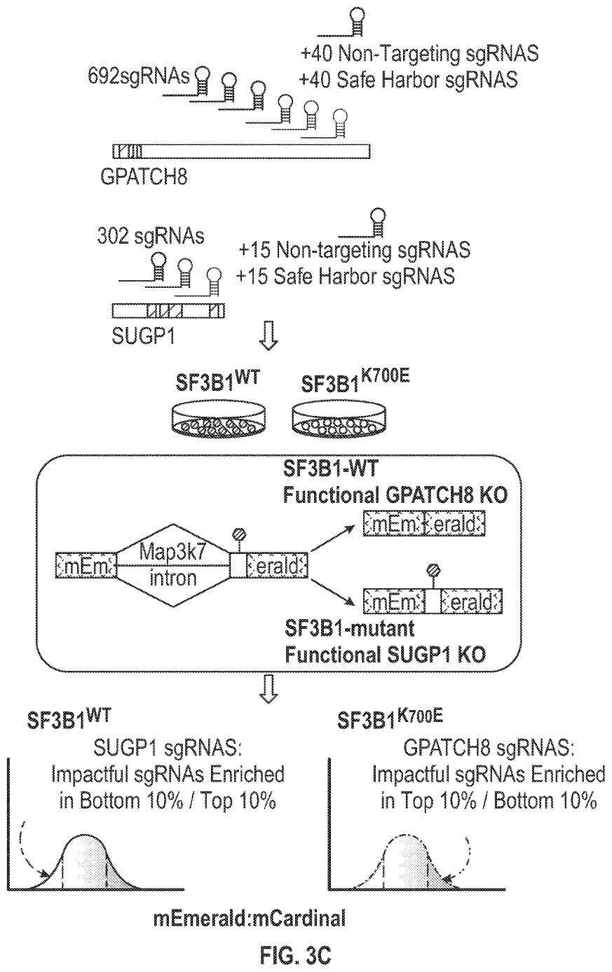

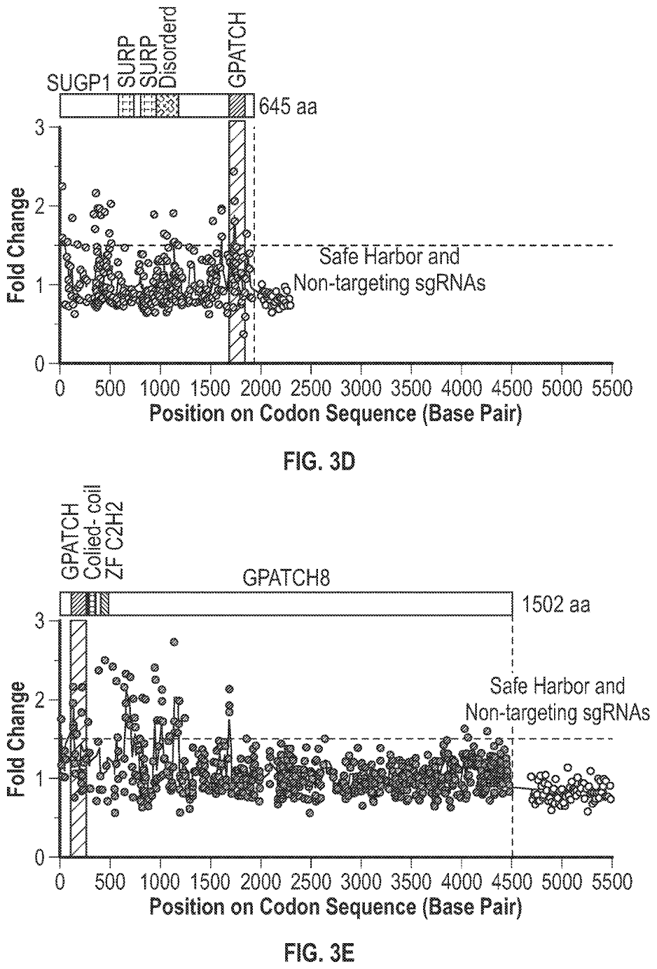

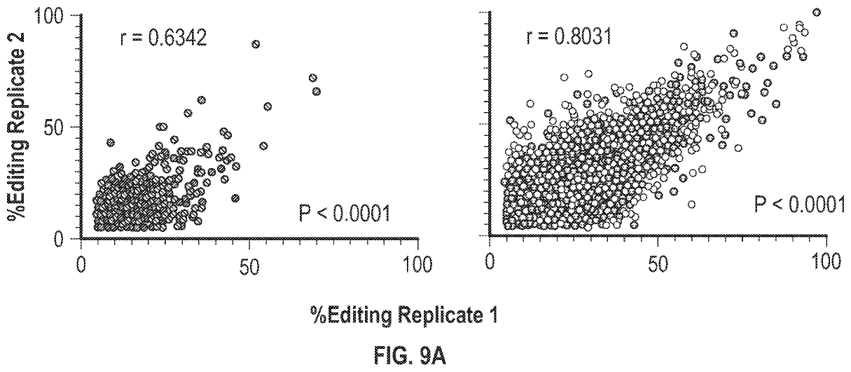

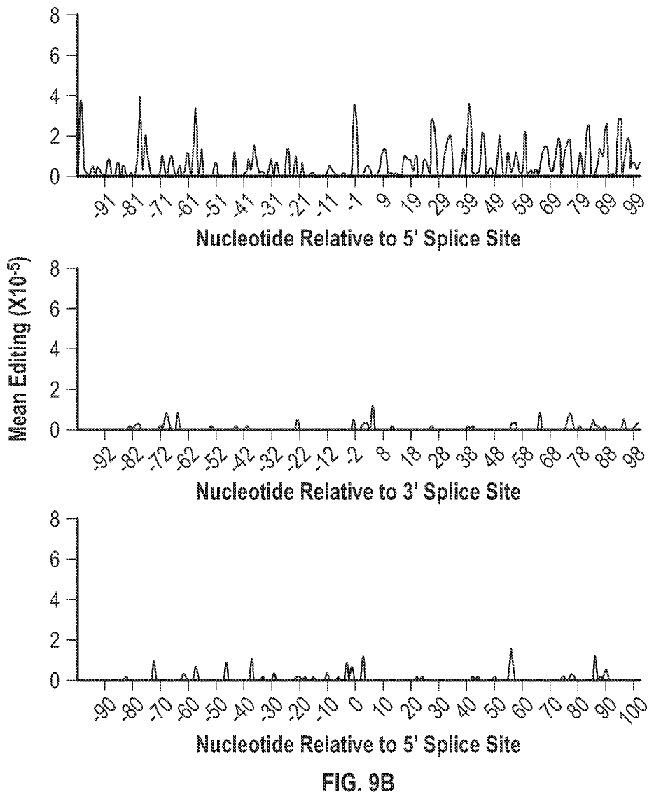

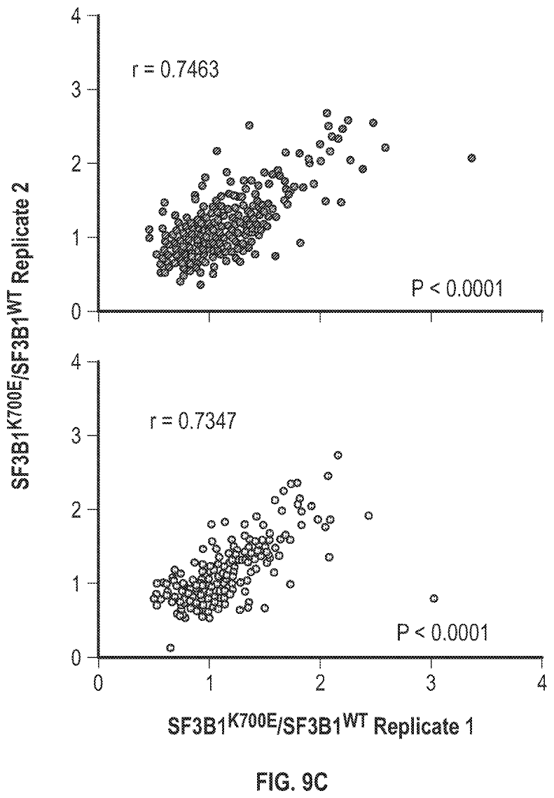

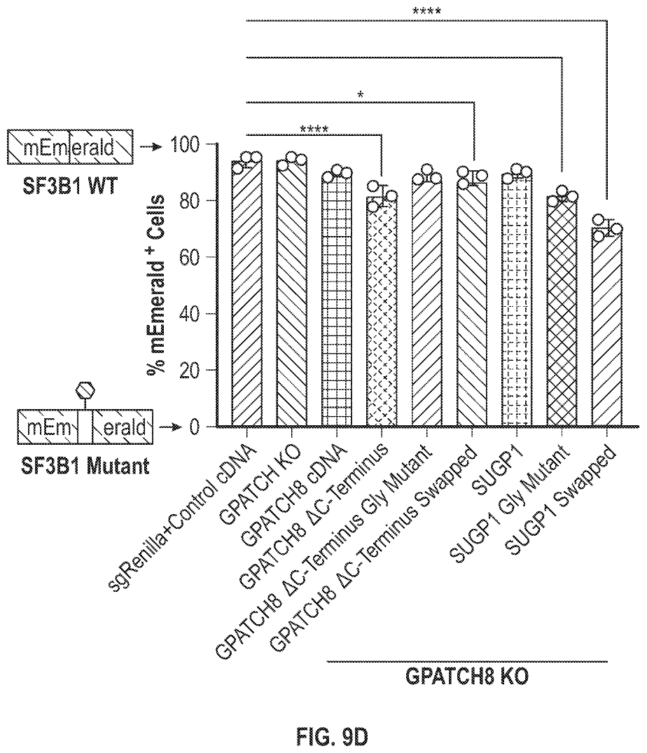

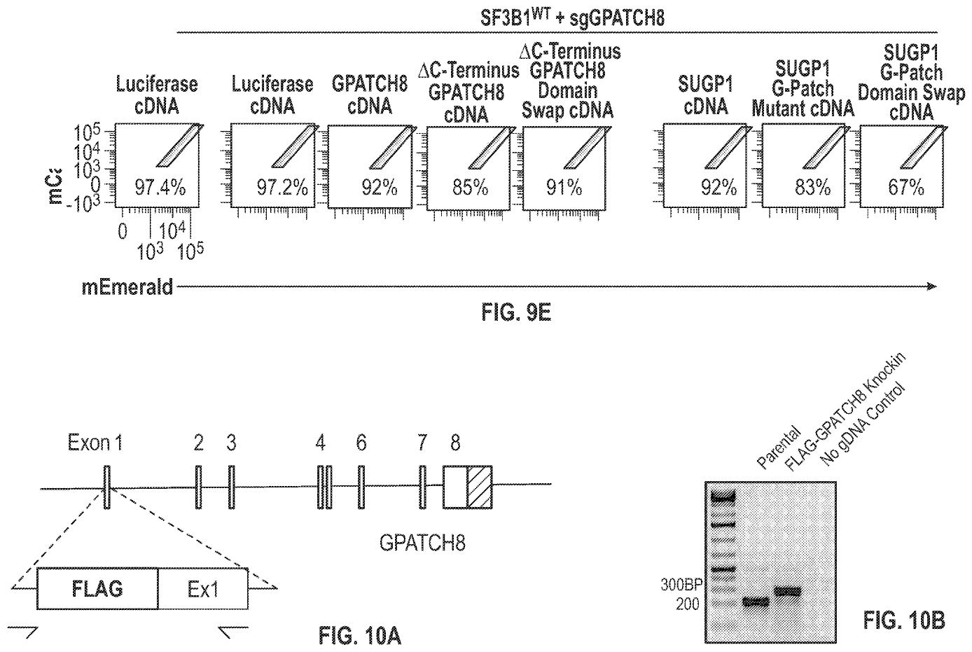

Given roles for GPATCH8 and SUGP1 in RNA splicing regulation, RNAs bound by each protein were evaluated. To accomplish this, the HyperTRIBE method was adapted to fuse GPATCH8 or SUGP1 to the catalytic domain of the Drosophila ADAR (Adenosine Deaminase Acting on RNA) enzyme. When expressed in cells, each fusion protein marks their binding sites with nearby A-to-I editing events thereby allowing for global mapping of the mRNA targets of each protein. Editing sites for both SUGP1 and GPATCH8 were most heavily enriched within introns (FIG. 3A and FIG. 9A). Interestingly for SUGP1, editing events clustered upstream of canonical 3′ss (FIG. 3B); however, this was not the case for GPATCH8 (FIG. 9B).

In addition to a G-patch domain, GPATCH8 also contains coiled-coil, C2H2 zinc finger, and SR domains, and the functional importance of each to GPATCH8 function are not yet known. To evaluate the functional importance of each domain of GPATCH8 and SUGP1 in regulating SF3B1 mutant mis-splicing events in a high-throughput manner, CRISPR sgRNA domain scanning of the coding region of GPATCH8 and SUGP1 was performed. A pool of 692 anti-GPATCH8 sgRNAs (with 40 non-targeting and 40 safe harbor negative control sgRNAs) as well as 302 anti-SUGP1 sgRNAs (with 15 non-targeting and 15 safe harbor negative control sgRNAs) was generated (FIG. 3C). This pooled series of sgRNAs were introduced into SF3B1 WT and mutant K562 cells bearing the MAP3K7 mEmerald split synthetic intron reporter and sgRNAs enriched in the top 10% and bottom 10% of mEmerald:mCardinal cells by FACS after two days of culture were sequenced. As such, sgRNAs with functional impact on SUGP1 RNA splicing activity would be expected to be enriched in the bottom 10% of fluorescent cells whereas sgRNAs with functional impact on GPATCH8 RNA splicing activity would be expected to be enriched in the top 10%. This experiment was performed in biological duplicate with good concordance (FIGS. 9B-9C) and highlighted the functional importance of G-patch domains in both SUGP1 and GPATCH8 (FIGS. 3D-3E). Moreover, the entire N-terminal portion of GPATCH8 consisting of the G-patch, coiled-coil, and C2H2 zinc finger, was critical for GPATCH8 regulation of SF3B1 mutant mis-splicing.

### Example 27

GPATCH8's G-Patch Domain is Required for Splicing Regulation and is not Interchangeable with SUGP1's G-Patch Domain

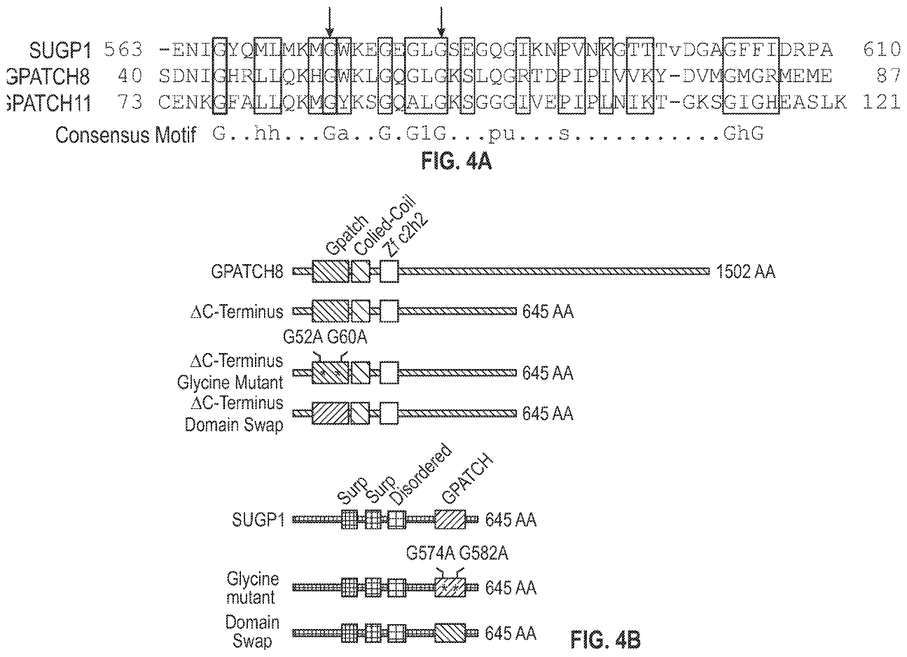

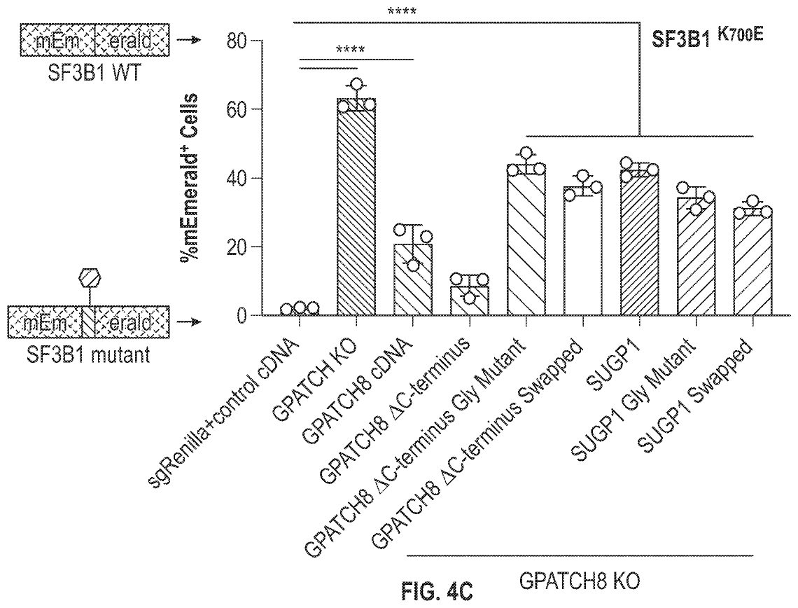

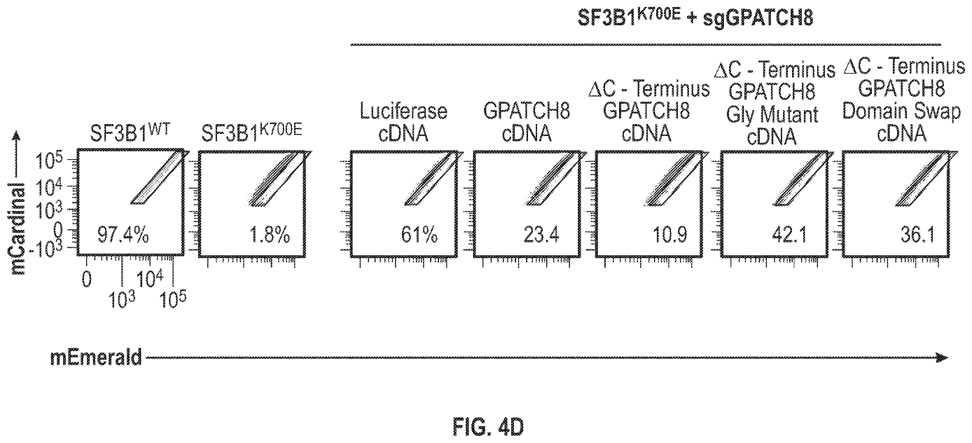

Prior work has identified that mutations in the G-patch domain of SUGP1 alone are sufficient to recapitulate the splicing alterations seen with mutant SF3B1. Given the homology of the G-patch domains between GPATCH8 and SUGP1 (FIG. 4A), it was next sought to delineate if specific residues of GPATCH8's G-patch domain were essential for its splicing activity and if the G-patch domains from each protein are interchangeable. To accomplish this, full-length GPATCH8 or a C-terminus truncated version containing only its coiled-coil, C2H2 zinc finger, and G-patch domains were expressed into the MAP3K7 synthetic intron reporter expressing K562 cells WT or mutant for SF3B1 (FIG. 4B). As shown earlier, SF3B1-mutant cells had virtually no mEmerald expression, while KO of GPATCH8 rescued mEmerald fluorescence (FIGS. 4C-4D). Reintroduction of full-length GPATCH8 into the SF3B1-mutant GPATCH8 KO cells suppressed mEmerald expression, underscoring the requirement of GPATCH8 for SF3B1 mutation-dependent mis-splicing. Interestingly, the N-terminus alone of GPATCH8 was sufficient to carry out this activity of GPATCH8. Moreover, mutating two conserved Glycine residues (GPATCH8 Glycine 52 and Glycine 60) within GPATCH8's G-patch domain greatly diminished GPATCH8's splicing activity. Additionally, replacing GPATCH8's G-patch domain with the G-patch domain of SUGP1 also greatly reduced GPATCH8's splicing activity. Similarly, expression of a version of SUGP1 whose G-patch domain was replaced with the G-patch domain from GPATCH8 was incapable of rescuing GPATCH8's splicing repression of the MAP3K7 synthetic intron reporter (FIGS. 4C-4D).

Performing the same series of above experiments with GPATCH8/SUGP1 constructs in SF3B1-WT cells revealed that expression of G-patch domain mutants of either SUGP1 (SUGP1 G574A-G582A) or GPATCH8 (GPATCH8 G52A-G60A) mimics SF3B1K700E-induced mis-splicing (FIGS. 9D-9E).

### Example 28

### DHX15 is the ATPase Ligand for GPATCH8

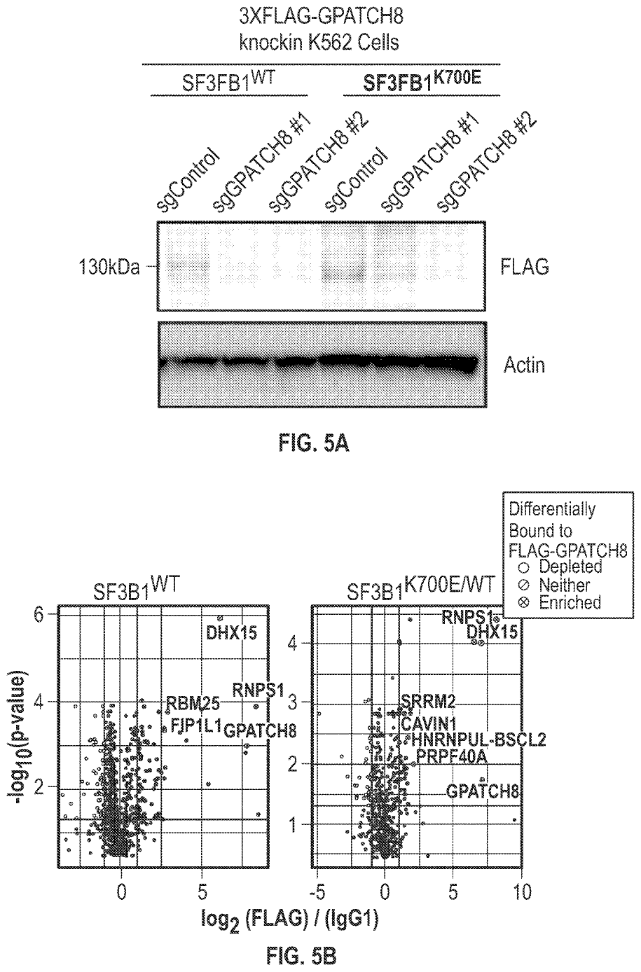

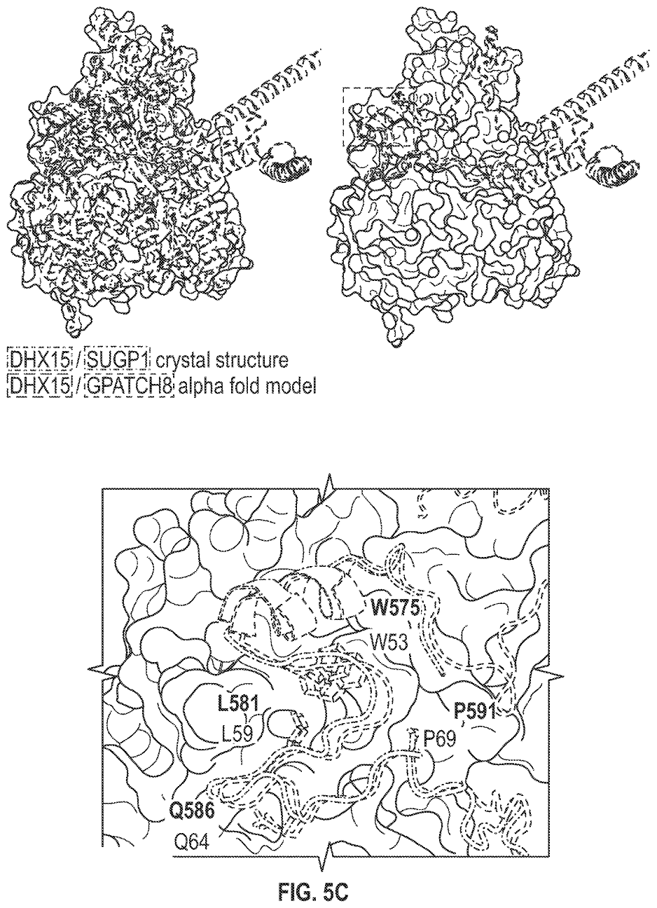

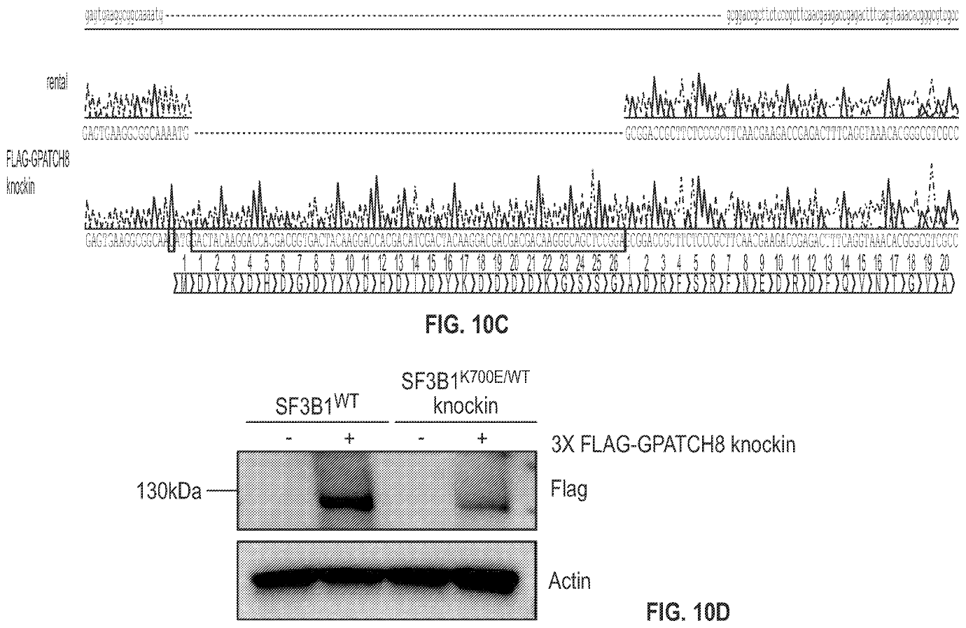

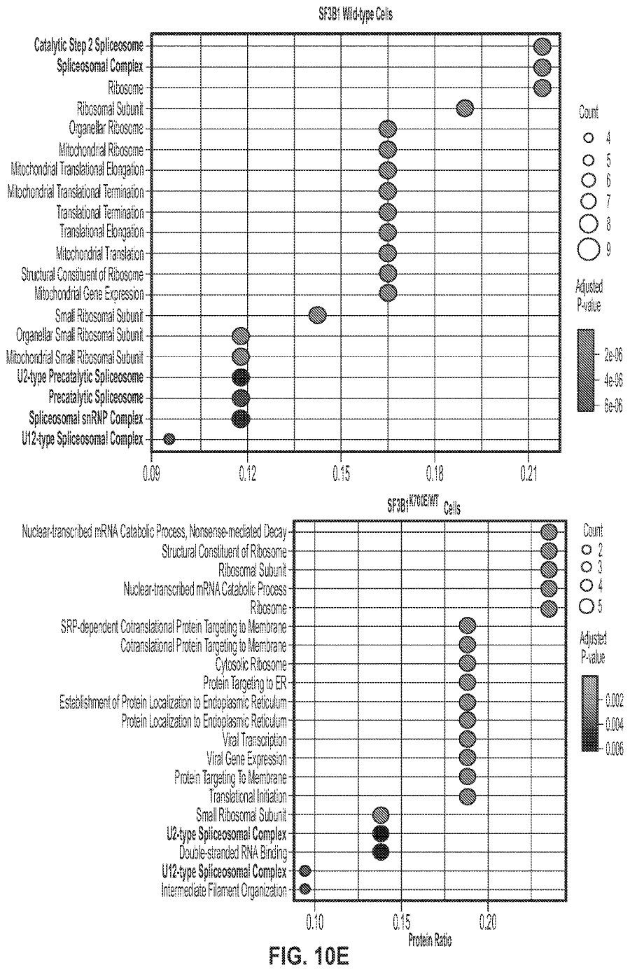

G-patch domains have been shown to enhance the affinity, helicase, and/or ATPase activity of RNA helicases. However, the target helicase remains to be identified for many of the >20 human G-patch proteins. To identify helicase binding partners of GPATCH8, endogenous GPATCH8 was purified from SF3B1-WT and -mutant cells. Given the lack of commercial antibodies which correctly recognize GPATCH8, the inventors knocked in the sequence encoding the FLAG epitope into the N-terminus of the locus encoding GPATCH8 in K562 cells WT for SF3B1 or with the SF3B1K700E mutation knocked in (FIG. 5A, FIGS. 10A-10D). Immunoprecipitation of FLAG in these cells followed by mass spectrometry in biological triplicate revealed GPATCH8 as a top protein, as expected, followed by robust interactions with the ATP-dependent helicase DHX15 in both SF3B1-mutant and -WT cells (FIG. 5B). Several additional U2 and U12 RNA splicing machinery components co-purified with FLAG-GPATCH8 as well (FIG. 10E).

As mentioned earlier, SUGP1 is known to interact with DHX15 as well and this interaction occurs via its G-patch domain. Recently, the co-crystal structure of the human DHX15-SUGP1 G-patch complex was published. Interestingly, mapping the predicted structure of the G-patch domain of GPATCH8 onto DHX15 identified that GPATCH8's G-patch domain interacts with DHX15 at the exact same location that DHX15 interacts with SUGP1 (FIG. 5C).

### Example 29

### Suppression of GPATCH8 Rescues Hematopoietic Defects of SF3B1 Mutant Hematopoietic Cells

Several prior studies have evaluated the functional importance of individual mis-splicing events generated by mutant SF3B1 in the biological phenotypes of SF3B1-mutant cancers. These include reports on the importance of MAP3K7 mis-splicing in the aberrant erythropoiesis of SF3B1-mutant hematopoietic malignancies, mis-splicing of the metabolic enzyme gene COASY as well as the metabolic hormone gene ERFE, and mis-splicing of the epigenetic regulator gene BRD9, amongst many others. Currently, however, there have been no attempts to correct mis-splicing of a larger number of SF3B1 mutation-induced mis-splicing events in parallel, as therapeutic means to achieve this do not exist.

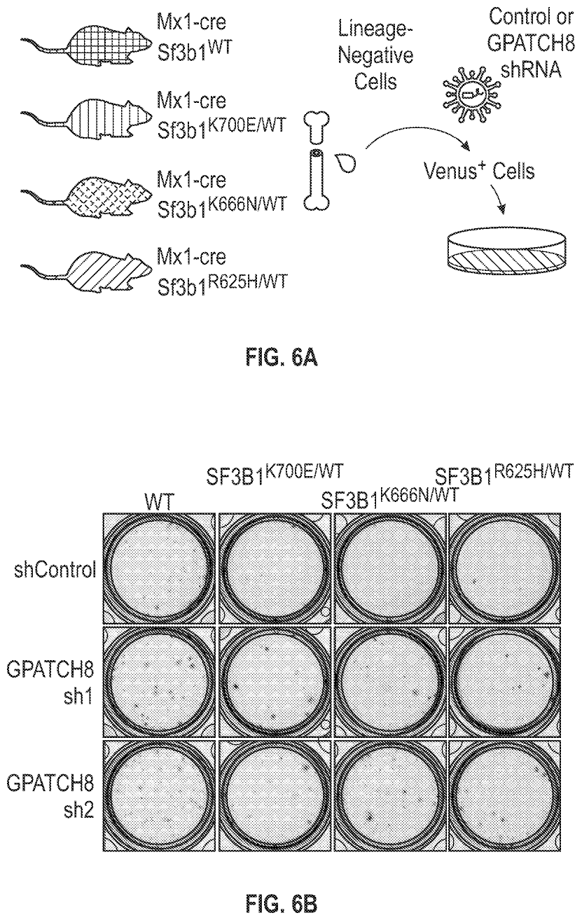

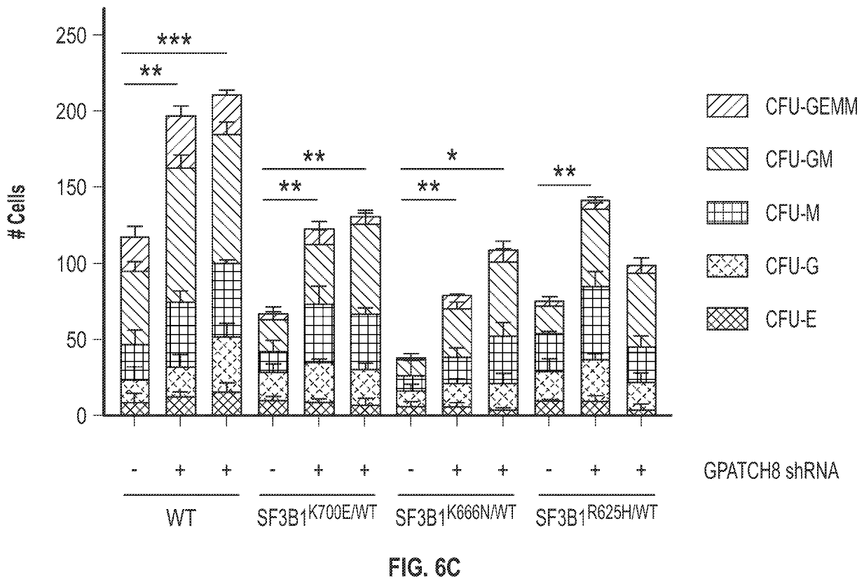

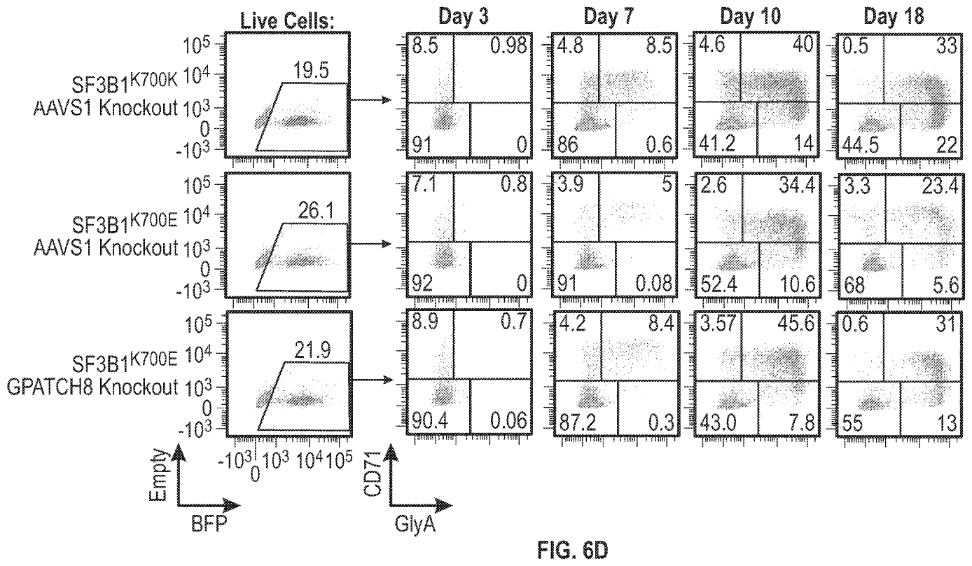

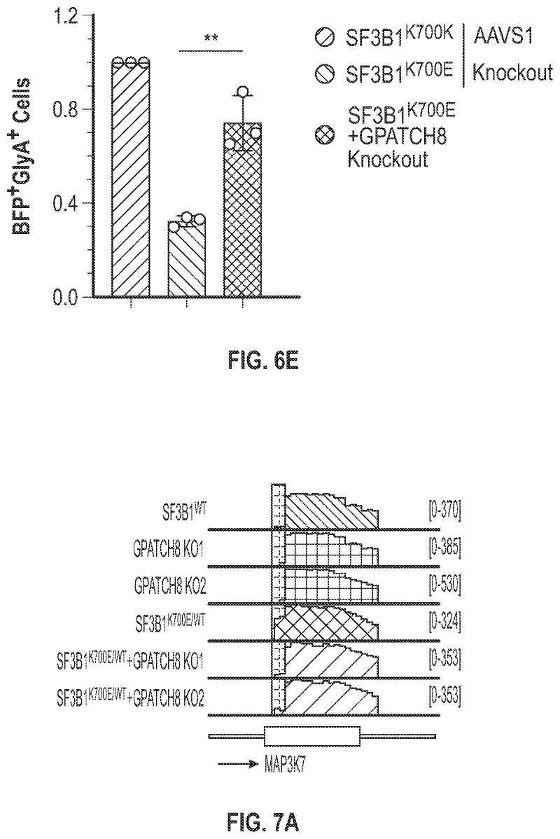

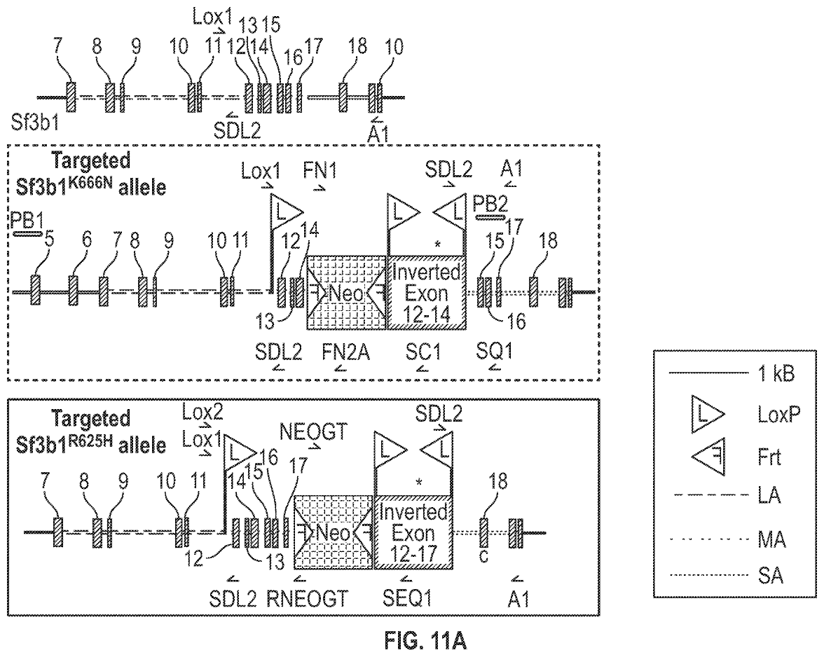

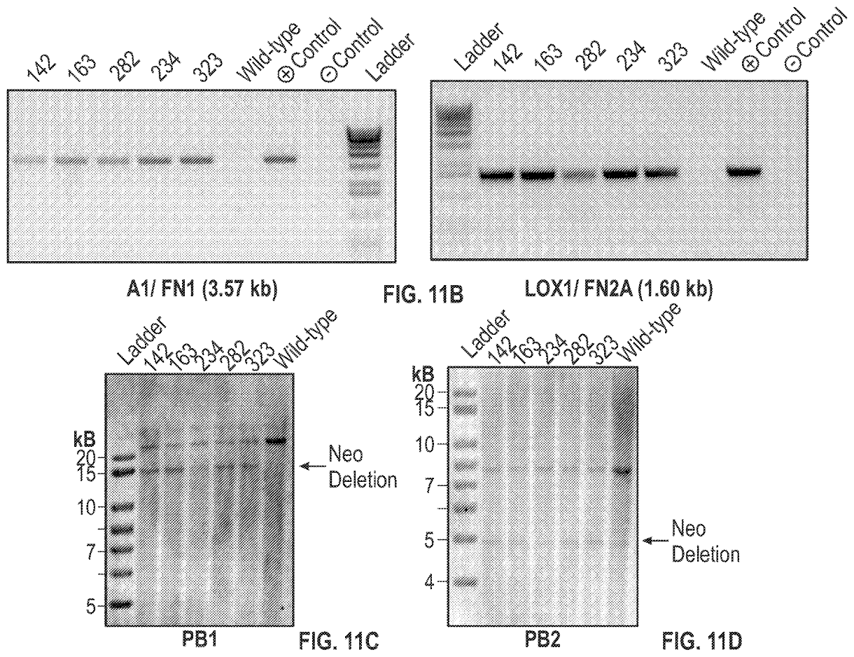

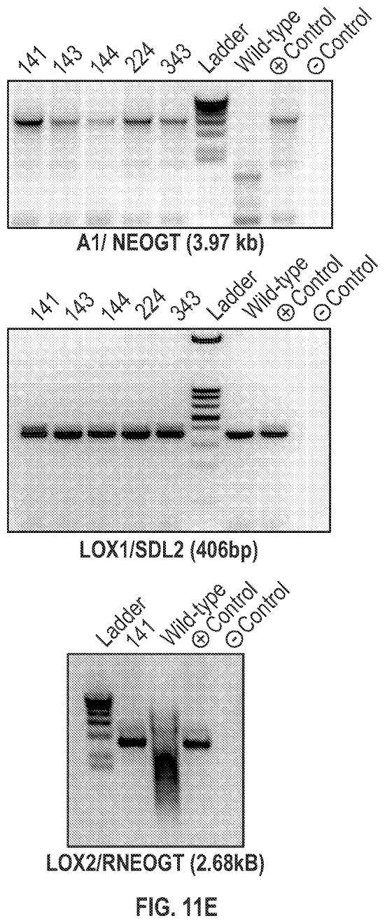

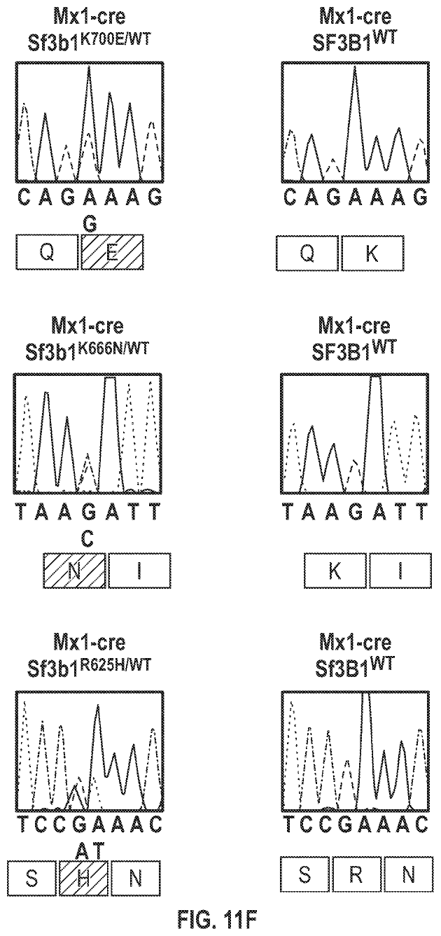

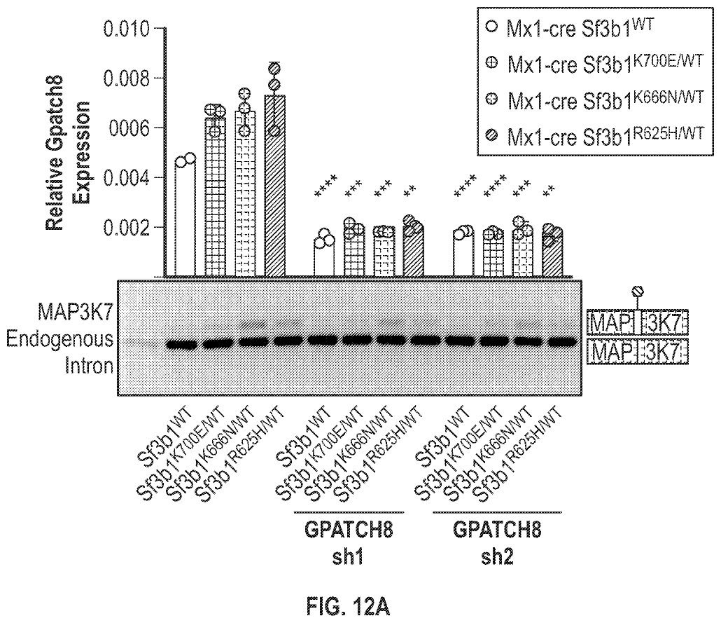

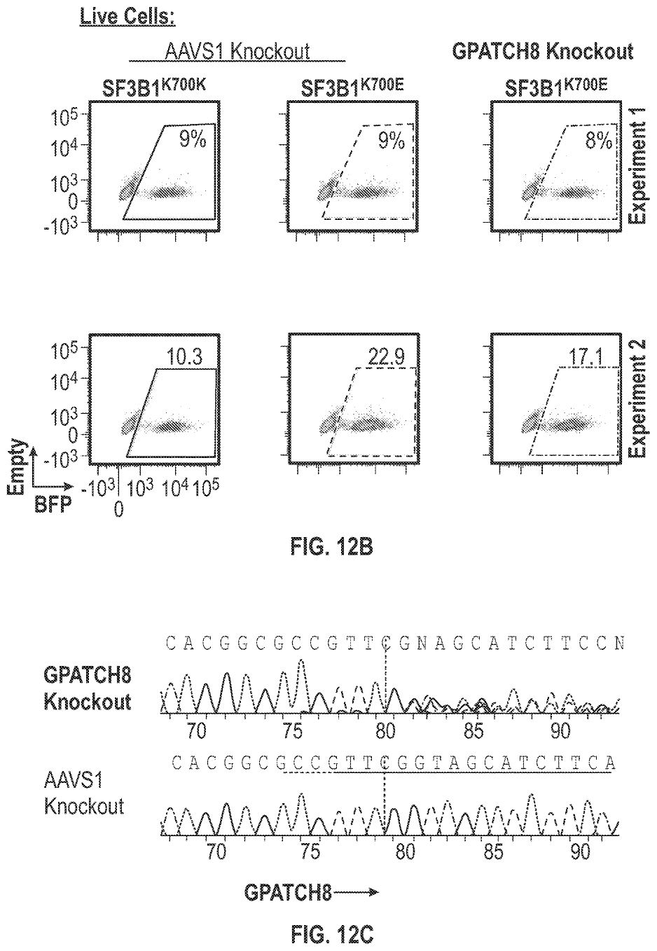

Given the inventors' discovery of GPATCH8 as required for a large fraction of SF3B1-mutant mis-splicing events, the impact of GPATCH8 KO on the impaired erythropoiesis of SF3B1-mutant hematopoiesis was evaluated next. To achieve this, new knockin mice with conditional expression of the Sf3b1K666N and Sf3b1R625H alleles to compliment previously generated Sf3b1K700E conditional knockin mice were generated (FIG. 11A). Upon Cre-recombination, these animals express each Sf3b1 mutation at the expected ~50% allele frequency from the endogenous Sf3b1 locus. Lineage-negative bone marrow (BM) hematopoietic cells from each model exhibited greatly impaired differentiation and clonogenic capacity in in vitro methylcellulose colony assays as well as the same Map3k7 mis-splicing as human cells (FIGS. 6A-6B, FIG. 12A). Importantly, however, silencing of Gpatch8 in these same cells prior to in vitro plating significantly rescued colony formation in each mutant-SF3B1 genotype as well as Map3k7 mis-splicing (FIGS. 6B-6C, FIG. 12A). Of note, Gpatch8 silencing was well-tolerated in WT mouse BM cells despite ~80% reduction in Gpatch8 expression.

Given potential differences in splicing and hematopoietic differentiation across mouse and human, the above murine studies were complimented with evaluation of the SF3B1 mutation and GPATCH8 deletion in human hematopoietic stem and progenitor cells (HSPCs). This was achieved by CRISPR editing with AAV6 homology-directed repair in human cord blood CD34⁺ cells to introduce the SF3B1K700E (or the control SF3B1K700K) heterozygous mutation and a BFP fluorescent reporter at the endogenous SF3B1 allele. This was combined with the deletion of either GPATCH8 or a non-targeting AAVSI control in the same cells. A mean of 16% SF3B1 precise editing efficiency marked by BFP⁺ and 58% GPATCH8 KO efficiency was achieved across CD34⁺ cells from three independent cord blood donors using this approach. Edited HSPCs were then differentiated into erythroid cells following an 18-day liquid culture (FIGS. 12B-12C). A defect in erythroid differentiation was evident in the SF3B1 mutant cells, consistent with the marked anemia in patients with SF3B1 mutant MDS (FIGS. 6D-6E). GPATCH8 KO partially corrected the erythroid defect, with the most pronounced rescue at the CD71⁺GlyA⁺ erythroblast stage in early erythropoiesis (FIGS. 6D-6E). Altogether, these data indicate that correcting SF3B1 mutant mis-splicing via GPATCH8 deletion ameliorates aberrant hematopoiesis associated with SF3B1-mutant myeloid neoplasms.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art. Publications cited herein and the subject matter for which they are cited are hereby specifically incorporated by reference in their entireties.

While illustrative embodiments have been illustrated and described, it will be appreciated that various changes can be made therein without departing from the spirit and scope of the invention.

SEQUENCES

SEQ ID NO: 1//Splicing factor 3B subunit 1
MAKIAKTHEDIEAQIREIQGKKAALDEAQGVGLDSTGYYDQEIYGGSDSRFAGYVTSIAATELEDD
DDDYSSSTSLLGQKKPGYHAPVALLNDIPQSTEQYDPFAEHRPPKIADREDEYKKHRRTMIISPERL
DPFADGGKTPDPKMNARTYMDVMREQHLTKEEREIRQQLAEKAKAGELKVVNGAAASQPPSKR
KRRWDQTADQTPGATPKKLSSWDQAETPGHTPSLRWDETPGRAKGSETPGATPGSKIWDPTPSHT
PAGAATPGRGDTPGHATPGHGGATSSARKNRWDETPKTERDTPGHGSGWAETPRTDRGGDSIGE
TPTPGASKRKSRWDETPASQMGGSTPVLTPGKTPIGTPAMNMATPTPGHIMSMTPEQLQAWRWE
REIDERNRPLSDEELDAMFPEGYKVLPPPAGYVPIRTPARKLTATPTPLGGMTGFHMQTEDRTMKS
VNDQPSGNLPFLKPDDIQYFDKLLVDVDESTLSPEEQKERKIMKLLLKIKNGTPPMRKAALRQITD
KAREFGAGPLFNQILPLLMSPTLEDQERHLLVKVIDRILYKLDDLVRPYVHKILVVIEPLLIDEDYY
ARVEGREIISNLAKAAGLATMISTMRPDIDNMDEYVRNTTARAFAVVASALGIPSLLPFLKAVCKS
KKSWQARHTGIKIVQQIAILMGCAILPHLRSLVEIIEHGLVDEQQKVRTISALAIAALAEAATPYGIE
SFDSVLKPLWKGIRQHRGKGLAAFLKAIGYLIPLMDAEYANYYTREVMLILIREFQSPDEEMKKIV
LKVVKQCCGTDGVEANYIKTEILPPFFKHFWQHRMALDRRNYRQLVDTTVELANKVGAAEIISRI
VDDLKDEAEQYRKMVMETIEKIMGNLGAADIDIIKLEEQLIDGILYAFQEQTTEDSVMLNGFGTVV
NALGKRVKPYLPQICGTVLWRLNNKSAKVRQQAADLISRTAVVMKTCQEEKLMGHLGVVLYEY
LGEEYPEVLGSILGALKAIVNVIGMHKMTPPIKDLLPRLTPILKNRHEKVQENCIDLVGRIADRGAE
YVSAREWMRICFELLELLKAHKKAIRRATVNTFGYIAKAIGPHDVLATLLNNLKVQERQNRVCTT
VAIAIVAETCSPFTVLPALMNEYRVPELNVQNGVLKSLSFLFEYIGEMGKDYIYAVTPLLEDALMD
RDLVHRQTASAVVQHMSLGVYGFGCEDSLNHLLNYVWPNVFETSPHVIQAVMGALEGLRVAIGP
CRMLQYCLQGLFHPARKVRDVYWKIYNSIYIGSQDALIAHYPRIYNDDKNTYIRYELDYIL
SEQ ID NO: 2//intron 1 of MTERFD3

| | |
|---|---|
| gtgagtcgcc cccttcctct gctcctgggc gtgttcctca ccagcggcgc cgcagcggtc | 60 |
| agggcccgca aaaccccacg cctcgccaga cgctcagctc agcctgccgg gtttctctgg | 120 |
| aaaagcggag gccccacagt gaatgtgcgg ccttttgatc ggtcctggag atcttccgtg | 180 |
| gaccctcgtt tttccttgct tataaacgtt ggtcaatttg aaactggcag cagggcatat | 240 |
| gtgttacgaa agaagaactt tattgaaacc agtggtggtg tagagaagac catactaacc | 300 |
| ataactgtgt attacagaag tgaatttcac tccccaaaaa tgcttaagaa acaagcatcc | 360 |
| tagaacagcc cacattgtga tttaacaaac atttaccaaa agcattgcta tatgtggggg | 420 |
| cacagggcta agagcaggca ggactctgga gccaacctgt ggggtttgag tccaaattct | 480 |
| gccggttttt tgttttttgt tttttttttt agctgagtaa ccttgggcaa gttcctttac | 540 |
| ctgcgtctgt ttcctcattt taaaatgagt tgttgtgatg atttaatgat ttagtatatg | 600 |
| tgaagagttt tctgcatatt ctaagtgatt tatggtatta actcgtttga tctttgcagt | 660 |
| cacccaggag atagtatcac cattttcctg atgccaaact gaggctcaga gaggtgaagt | 720 |
| aacttcttca agtcacaaca caactgttgt ccaggaatca ggcagtctgg ctctgctaac | 780 |
| ccagttttac ccactgcatt aatgtaccgc atccacttgt tcactcagtt tcacctgaca | 840 |
| aatactaatt ttgtctctct gtggaacaag ttctttgctg ggtgcaggga ataaagcaga | 900 |
| acaccgttca gttcctgcct tcccaacgct tgtaaatcta gtgcaggtca ttcatggccg | 960 |
| ggctgcaggg tgtccatgaa tcctgctaaa atcatttgcg aaatgttgtg agcgtgggtc | 1020 |
| ttttcttctg gaaagagagt ctgtgactct caggttctga gttgatctgt gatccaaaca | 1080 |
| gaggtgtgaa gaagtgttgt tttagcagat ctgtgcctag tatgcctgat acacagcttg | 1140 |
| aacttcaaac aaacagcaac aaaattccta ctggttggaa gaatgaagaa cttgtaagag | 1200 |
| cataataagt gctgcagtag cgtccggagc agggtgctgt ggaagagcac acagaaggcc | 1260 |
| acctgcccag gaatgcctcc tagaggcagt gaaggcagct gggtgaagga tagggaagca | 1320 |
| gttcattgga acactgtggt gaccagtctg tccatgtagt gttgagacag aaacaacacg | 1380 |
| tgaacagctg cagctgttgt accacttgtg cgcaagtgta aaagtccatg gtgccatgaa | 1440 |
| atcatgtaat ggggacagga ggcttcatgt attcgtgaag gttagaaaag tttccttgag | 1500 |
| aaagtgacac ttaggcctaa aagttggagc attttgggca tggaatctgg cccacagtct | 1560 |
| tcaaagacca catggtagaa accagtagag caccagggac tgacaggaag caaatgcagc | 1620 |
| tggtgcagga gagggaaatg ggaattaggg tggtggcaga gcccaaagag gccttgtagg | 1680 |
| ccatggtaag gcatttctat gttttatttt actttgtctt tatcctaaaa tgccattggc | 1740 |
| aagtttattg cag | 1753 |

SEQ ID NO: 3//intron 4 of MYO15B

| | |
|---|---|
| gtgagtggtg gccttcctgg gtggctgaac ccatgggcca cactgagggt tgatggggcg | 60 |
| gggcctgagg tcttcactgg aagccactgg tatgctccac tgctgggcta cagttctgga | 120 |
| ctcctcaggg ccgagctcct ggctgaaatc atcacatgag cccctggaat gtggcatctt | 180 |
| ggggactttt tcttttctgt cgggtctctc catgccctgg gactatcttt ccacatttat | 240 |
| gggggtctct ccaggctagc tgtcacttct gggtggcaca tcccacttcc tgggttcact | 300 |
| ggggtggtcc tatgtgcccc tccctggaga ccctaccaac caacacatcc ttagctcctc | 360 |
| ccactgcag | 369 |

SEQ ID NO: 4//intron 10 of SYTL1

| | |
|---|---|
| gtgaggctgt gaccacgatg cggttccccg ttaatgaact ggacgccccc ttcctgcggg | 60 |
| gctaggtggc aagggcagcc agtaacgtca ttgcccggag gatcggcgga gggggcccat | 120 |
| taactcgtta tccagtgttg tcagcctcgt ggtgggcgtg gtgatagtgc aggtccccat | 180 |
| taatgccctt aggggctccc cagaattcca tcatggtagg aacgcggtag gacctgcctc | 240 |
| agccaactcg cgcagcatct acgcgggcca ccagcagtgc tccactaaag ctcacctcct | 300 |
| gtcctcag | 308 |

SEQ ID NO: 5//intron 11 of SYTL1

| | |
|---|---|
| gtgaggcagc caggccgcgt ggggagacct gcggcccggg tctcctgcat ttaccccacc | 60 |
| aggctctccc gcagccccct cacaccccgc cttcgacaga acctcccctc aacctcttaa | 120 |
| cctcatggcc ccaggcgaag cccggccggc cacggcccct tccccgaggg cgctaggacc | 180 |
| cctaggttct gcccctgcag gccccgccgt ctcttctagc cgcaccccat ccgggtctgc | 240 |
| agaccccacc ctcctgaggc ccctttccat tagcccctgc tccacgataa gcccgcctct | 300 |
| cgcag | 305 |

SEQ ID NO: 6//intron 4 of MAP3K7

| | |
|---|---|
| gtgagtgtca ttagacctgt ctttatctag tggattaaaa taatttgaaa aattttaata | 60 |
| taaaccctaa gttgtttaac attcttcaca atttgccgtc agagtcccaa aagggcataa | 120 |
| tttttaaaaa tctgctaaga ataaaaatgg aaagataaga ttctataaca ttagtatgtg | 180 |
| aaatttagag gtctatcaat tttttaagta gatggtactt tgcatttcat taaaagcatt | 240 |
| taagtaaaag tttacttttt aaatagattt taaaatatct tcagattttt ttctttttgc | 300 |
| cctctcataa ctttgagcct cctcatttta gaattcttta accatatttc gatttcactc | 360 |
| tgctttattt tttctttttt ttcaaaaaaa aatgtgaaaa ctcacttcac acaacctttt | 420 |
| atttgtagta atgaaaaaaa actttgtgta gttacattgg agaacagagt tcttttaccc | 480 |
| taaaaatacc aaccatatgc agaaatgtat tgtctcttgt agcaccagtt atataaaaca | 540 |
| aattgttaca ttttattttt taattagtat tattttatgt aaacttttac ttatatttac | 600 |
| tgcccatgtg ttcatcattg tgatggatat ctaatacccc attgtcttat ttgattgaac | 660 |
| tacttctggt ttttagccat tacagaaaat acaggtgcaa acatcttgat atacattgtt | 720 |
| ttgtttcttt ctgtactgtt ctctgcctct ctcttgtagt agtgtgccat aagactgctc | 780 |
| ataatttata ttactttata aaagtcttta tctgtttgaa aaaatgtggt aatgtatatt | 840 |
| gattttactt caatgtagga ataacattta atagatgtac tattaaatgt taatatcatg | 900 |
| gcaattatcc actcaaaatt agtagatatt ttcatccttt ttatttagaa gaaaaattaa | 960 |
| aaggccaggc atggtggctc actcctgtaa tcccagcact ttgggaggcc aaggcaggca | 1020 |
| gatcatgagg tcaagagatc aagaccatcc tggccaacat ggtgataccc tatctctact | 1080 |
| aaaaatacaa aaattagctg ggcgtggtgg cacacgcctg tagtcccagc tactcgggag | 1140 |
| gctgagccag gagaatcgct tgaacccagg aggtggagtt tacagtgagc caagatcgca | 1200 |
| ccactgcact ccagcctggc gacaaagtga gagtccgtct caaaaaacaa aaagaaaaaa | 1260 |
| gaaagtaaaa aattaaaagc aggactgatg gagcatatgt atatgtagaa aaatacccag | 1320 |
| tcatgtgttc tgatagtgct tgcattcaca ttgtgtcttt tgttatatgc aataaagttc | 1380 |
| tttttagttt gtgcctttct ttcgcag | 1407 |

SEQ ID NO: 7//intron 1 of ORAI2

| | |
|---|---|
| gtgagtgtgg cggcgcgggg gcctggcgcg gggagcgggg tccctgcggc tgggggggcc | 60 |
| ggaattggac cggggacgct cggcggggaa ggggacgact gtaggctgcc cgggcgcggt | 120 |
| cactacccat ccggggttgg tgacctccac cccgcgggat ccctggcggg gtgcggcgcc | 180 |
| cagaggatca gggtcggcgg cgcagccggt tccggacacc ggggcgtccc tgggggaggg | 240 |
| acagggatgc agcctagcgc ccctgggagg ccccctccca gtgcgcctgc tggagagaga | 300 |
| cactcgccat tgccggtgcc ccagatgctt agaggaagtc aaactccaag accgccctct | 360 |
| ggaaatgtgt ccagtaggcc agccaggaag aaaagctcat ctctagagag gggatcgcgt | 420 |
| tattctacaa gacctgtttt ctcggactgc tcattaaagc aaaaaaaaaa aaaaaaaaaa | 480 |
| aaaagcaacc ccacaaggtt cagtcccgga gcatcctcta gggtctgtgc cacctgacag | 540 |
| aagtacaggg ctccatcctg ccccacccca ccccctgcct gggatccagt gtgaggccca | 600 |
| gactctgtcc ctccccctgg gccaccgggc ctggcacaca cagttggcat tgggaacata | 660 |
| aaggatggat ttgaaccccc tcctgggctg ctccttcgag acctggagac ctgtctcaag | 720 |
| acccagctcc aggcagacag acatcgtttc ctccacaatc ccacccactc tggggacatc | 780 |
| tgcagggcct caccgatggg gcatgggagg cctggcggct cccaggaagc caccctcagc | 840 |
| cctcactgcc ctgagctctt cacctccact ctcagacctg agcccagccc acccttactg | 900 |
| atggggcatc cctcttgttc acccttcctt cctttggagg ccaccaggct ctcccttgcg | 960 |
| tcccactcct gccagtcggc cagccatttc cctgggttga actgtggtcc caaaccagaa | 1020 |
| tgatctagca gtgcccagac cgttccactc accaatttct tccattctcc tgtggctgga | 1080 |
| cgcccagtca tgctgcccag accctctggc tcattccaca gccccaggct ccaagtgtct | 1140 |
| tccacccaca tctccctgcc ccctccaccc cccaggtggc agccagtctt ccctccatat | 1200 |
| ctcctggaca aggagagcca ccccacatgt acacctggag tggccttccc gtctctcgga | 1260 |
| atccttccat gcaggcagca tcttgtcttt gaggaaaggt ccctcttttt taaaattctt | 1320 |
| tttagaggcc aggcgcagtg gttcatgcct ataatctcag cactttggga ggccaaggtg | 1380 |
| ggcggatcac ttgagctcag gagttctaga ccagcctggc caacatagca aaaccctgtc | 1440 |
| tctaccaaaa ataccaaaaa ttagccaggt atggtggcac acacatgtag tcccagctac | 1500 |
| tcgggtggtt gaggcgggag gatcgcttga gcctgggagg tagaatttgc agggagctga | 1560 |
| gattgtgcca ctgcactcca gccttggtaa cacagcagga ccccatgtca aaaaaaatta | 1620 |
| tttttagaga cagcgtctta ctctgttgcc taggctggtc tccagctcct ggccgcaggt | 1680 |
| gatcctccca ccttggcctc acaaaacact gggattacag gcatgaggca ctttggcatg | 1740 |
| caaagggccg ctctgtcccc tccccggtgt tacctgcaag caccttttcc tggccttggt | 1800 |
| gtgaggcagc cagtcctctg ctccctgccc tccattgaag cctttagctc agtggatgag | 1860 |
| gattgccccc ccctcttttt tttttttttt tttgagatgg agtctcgctc tgttgcccag | 1920 |
| gctggagtgc agtggtgtgc tcttggctca ctgcaacctc tgccaccaca cccagctatt | 1980 |
| tttttttttt taagacagag tctcactgtg tcacccaggc tggagtgccg cggtaggatc | 2040 |
| tcagctcact gcaacctccg cctcctggga tcaggcaatt ctcctgcctc agcctcctga | 2100 |
| gtagctggga ttacaggcgc ccaccaccat gcccagttac tttttgtatt tttagaagag | 2160 |
| acaggtttcc accatgttgt ccaggctagt ctcgaactcc tgacctctag tgatccgccc | 2220 |
| acctcagcct cccaaagatg acaggtgtga gctactgctc ctgacccaca tccccttacc | 2280 |
| ttgctcccct actgggaggc catgaattct ggcctctttc atcttggatc tttaagtacc | 2340 |
| aagtgcgcta agcacctgtg tgatggagga gggaggacgg agggagcaaa ggttagcgtc | 2400 |
| tcacccctcc agtctctgta tactgcggtc atcagacctg catgctcaat tctgttaact | 2460 |
| atgggggtgg ggggagagga aaaatgcagt ttccaaatta cagcattttc tattatgtgg | 2520 |
| agctaaccta cggattgcag cgtttctctg aattctcccc aag | 2563 |

SEQ ID NO: 8//intron 1 of TMEM14C

| | |
|---|---|
| gtgcgagtat ttggggatta ttcttatttt ctgccacttt taacttttag ggattattta | 60 |
| ggagtttcac ggccgtctgc ttttcgtccc cccgattcag cgggccttca ggccgtgtgg | 120 |
| tcggtgcttt tctcgttggg tattttctgc ttttaaaaga agttgtggat gcgcggagcc | 180 |
| cctgggctcc tgaggcaaag gcttacccat gtagcatagt gttgcctcgt ttcttggtga | 240 |
| ttttcttgct ccatctcttt aaaagcctcc cttactcggt gccgtctcga gttaagaact | 300 |
| gtgggcaaga tcccaagccc gctgcccttc cctgttttat ggaaacttaa ttcttttttt | 360 |
| tttttttttt gtccttgagg gagggacttg ctctgtcgcc catgcttgag tgcagttgcc | 420 |
| tgaatatgtc tcattgcagc ctctgcctcc tgggctcaag ccatcctccc acttcagcct | 480 |
| cccaagtagc tgggatttac agtcgcatat caccatgcgc gattaatttt tttatatttt | 540 |
| ttagtagaca atgggtttca ctatgttggc caggctggtc ttgaactcct aaccccaggt | 600 |
| gatccgcccg cctcggcctc ctaaagtact gggatgacag gcgtcagcca ctgcgcttgg | 660 |
| cctaattctt taaacagaat aaacggggta tgcattgctt tcatcttttg gctcactgga | 720 |
| cacaggatac tttctgtaag aaaatagaag ctgtttttcc aagggtgtag tgtcacatgt | 780 |
| gaatatgacc actgtttccg tatattttat cctctcctac tactgccctc ctaacaagaa | 840 |
| ctgtgagttg gacgcagaag tttctaaaaa agttgagctt tgaaattggc tgttgcagca | 900 |
| gggatgaaaa gcaacacccc tacctcccct caaaagagac attaaagtag ttggattaag | 960 |
| ggcacgggag tatttgcttt tgaatttagt gataacatgg gtagctgatg aaatgactaa | 1020 |
| cacattccct gattttagag ctggtcagtg gatcttgctg agtttcccgt gggcctatgt | 1080 |
| gattaaaact gaggttttca tgacaatggt cagcatctgt tcagggttta ctaggtgcta | 1140 |
| agcaccttta catgtgacat ccattgaatg ctcactacag ccccaggaaa tcggtaccag | 1200 |
| tgttatcctc atggtacagt gaagaatact gagacttagg ttgcgtagct tgcaggttgg | 1260 |
| acacacttct ttctgactgc tggagagctg tgcttttaac tacctctgat ccagcttgtt | 1320 |
| ttctgcag | 1328 |

SEQ ID NO: 9

| | |
|---|---|
| gtgagtcgcc cccttcctct gctcctgggc gtgttcctca ccagcggcgc cgcagcggtc | 60 |
| agggcccgca aaaccccacg cctcgccaga cgctcagctc caggaagcaa atgcagctgg | 120 |
| tgcaggagag ggaaatggga attagggtgg tggcagagcc caaagaggcc ttgtaggcca | 180 |
| tggtaaggca tttctatgtt ttattttact ttgtctttat cctaaaatgc cattggcaag | 240 |
| tttattgcag | 250 |

SEQ ID NO: 10

| | |
|---|---|
| gtgagtcgcc cccttcctct gctccgagag ggaaatggga attagggtgg tggcagagcc | 60 |
| caaagaggcc ttgtaggcca tggtaaggca tttctatgtt ttattttact ttgtctttat | 120 |
| cctaaaatgc cattggcaag tttattgcag | 150 |

SEQ ID NO: 11

| | |
|---|---|
| gtgagtcgcc cccttaggcc ttgtaggcca tggtaaggca tttctatgtt ttattttact | 60 |
| ttgtctttat cctaaaatgc cattggcaag tttattgcag | 100 |

SEQ ID NO: 12

| | |
|---|---|
| gtgagtggtg gccttcctgg gtggctgaac ccatgggcca cactgagggt gtcttcactg | 60 |
| gaagccactg gtatgctcca ctgctgggct acagttctgg actcctcagg gccgagctcc | 120 |
| tggctgaaat catcacatga gcccctggaa tgtggcatct tggggacttt ttcttttctg | 180 |
| tcgggtctct ccatgccctg ggactatctt tccacattta tgggggtctc tccaggctag | 240 |
| ctgtcacttc tgggtggcac atcccacttc ctgggttcac tggggtggtc ctatgtgccc | 300 |
| ctccctggag accctaccaa ccaacacatc cttagctcct cccactgcag | 350 |

SEQ ID NO: 13

| | |
|---|---|
| gtgagtggtg gccttcctgg gtggctgaac ccatgggcca cactgagggt tgatggggcg | 60 |
| gggcctgagg tcttcactgg aagccactgg tatgctccac tctctccatg ccctgggact | 120 |
| atctttccac atttatgggg gtctctccag gctagctgtc acttctgggt ggcacatccc | 180 |
| acttcctggg ttcactgggg tggtcctatg tgcccctccc tggagaccct accaaccaac | 240 |
| acatccttag ctcctcccac tgcag | 265 |

SEQ ID NO: 14

| | |
|---|---|
| gtgaggctgt gaccacgatg cggttccccg ttaatgaact ggacgccccc gggctaggtg | 60 |
| gcaagggcag ccagtaacgt cattgcccgg aggatcggcg gagggggccc attaactcgt | 120 |
| tatccagtgt tgtcagcctc gtggtgggcg tggtgatagt gcaggtcccc attaatgccc | 180 |
| ttaggggctc cccagaattc catcatggta ggaacgcggt aggacctgcc tcagccaact | 240 |
| cgcgcagcat ctacgcgggc caccagcagt gctccactaa agctcacctc ctgtcctcag | 300 |

SEQ ID NO: 15

| | |
|---|---|
| gtgaggctgt gaccacgatg cggttccccg ttaatgaact ggacgccccc ttcctgcggg | 60 |
| gctaggtggc aagggcagcc agtaacgtca ttgcccggag tggtgatagt gcaggtcccc | 120 |
| attaatgccc ttaggggctc cccagaattc catcatggta ggaacgcggt aggacctgcc | 180 |
| tcagccaact cgcgcagcat ctacgcgggc caccagcagt gctccactaa agctcacctc | 240 |
| ctgtcctcag | 250 |

SEQ ID NO: 16

| | |
|---|---|
| gtgaggcagc caggccgcgt ggggagacct gcggcccggg tgcatttacc ccaccaggct | 60 |
| ctcccgcagc cccctcacac cccgccttcg acagaacctc ccctcaacct cttaacctca | 120 |
| tggccccagg cgaagcccgg ccggccacgg ccccttcccc gagggcgcta ggacccctag | 180 |
| gttctgcccc tgcaggcccc gccgtctctt ctagccgcac cccatccggg tctgcagacc | 240 |
| ccaccctcct gaggcccctt tccattagcc cctgctccac gataagcccg cctctcgcag | 300 |

SEQ ID NO: 17

| | |
|---|---|
| gtgaggcagc caggcaggct ctcccgcagc cccctcacac cccgccttcg acagaacctc | 60 |
| ccctcaacct cttaacctca tggccccagg cgaagcccgg ccggccacgg ccccttcccc | 120 |
| gagggcgcta ggacccctag gttctgcccc tgcaggcccc gccgtctctt cccatccggg | 180 |
| tctgcagacc ccaccctcct gaggcccctt tccattagcc cctgctccac gataagcccg | 240 |
| cctctcgcag | 250 |

SEQ ID NO: 18

| | |
|---|---|
| gtgagtgtca ttagacctgt ctttatctag tggattaaaa taatttgaaa aattttaata | 60 |
| taaaccctaa gttgtttaac attcttcaca atttgccgtc aaagaaagta aaaaattaaa | 120 |
| agcaggactg atggagcata tgtatatgta gaaaaatacc cagtcatgtg ttctgatagt | 180 |
| gcttgcattc acattgtgtc ttttgttata tgcaataaag ttctttttag tttgtgcctt | 240 |
| tctttcgcag | 250 |

SEQ ID NO: 19

| | |
|---|---|
| gtgagtgtgg cggcgcgggg gcctggcgcg gggagcgggg tccctgcggc tgggggggcc | 60 |
| ggaattggac cggggacgct cggcggggaa ggggacgact ctctgtatac tgcggtcatc | 120 |
| agacctgcat gctcaattct gttaactatg ggggtggggg gagaggaaaa atgcagtttc | 180 |
| caaattacag cattttctat tatgtggagc taacctacgg attgcagcgt ttctctgaat | 240 |
| tctccccaag | 250 |

SEQ ID NO: 20

| | |
|---|---|
| gtgcgagtat ttggggatta ttcttatttt ctgccacttt taacttttag ggattattta | 60 |
| ggagtttcac ggccgtctgc ttttcgtccc cccgattcag agccccagga aatcggtacc | 120 |
| agtgttatcc tcatggtaca gtgaagaata ctgagactta ggttgcgtag cttgcaggtt | 180 |
| ggacacactt ctttctgact gctggagagc tgtgctttta actacctctg atccagcttg | 240 |
| ttttctgcag | 250 |

SEQ ID NO: 21

| | |
|---|---|
| gtgagtcgcc cccttcctct gctccgagag ggaaatggga attagggtgg tggcagagcc | 60 |
| caaagaggcc ttgtaggcca tggtaaggcg tttctgtgtt ttgttttgct ttgtctttat | 120 |
| cctaaaatgc cattggcaag tttattgcag | 150 |

SEQ ID NO: 22

| | |
|---|---|
| gtgagtcgcc cccttcctct gctccgagag ggaaatggga attagggtgg tggcagagcc | 60 |
| caaagaggcc ttgtaggcca tggtaaggct actaacaatt tctatgtttt attttacttt | 120 |
| gtctttatcc taaaatgcca ttggcaagtt tattgcag | 158 |

SEQ ID NO: 23

| | |
|---|---|
| gtgagtcgcc cccttcctct gctccgagag ggaaatggga attagggtgg tggcagagcc | 60 |
| caaagaggcc ttgtaggcca tggtaaggca tttctattac taacagtttt attttacttt | 120 |
| gtctttatcc taaaatgcca ttggcaagtt tattgcag | 158 |

SEQ ID NO: 24

| | |
|---|---|
| gtgagtcgcc cccttcctct gctccgagag ggaaatggga attagggtgg tggcagagcc | 60 |
| caaagaggcc ttgtaggcca tggtactaac ataaggcgtt tctgtgtttt gttttgcttt | 120 |

SEQ ID NO: 25

| | |
|---|---|
| gtgagtcgcc cccttcctct gctccgagag ggaaatggga attagggtgg tggcagagcc | 60 |
| caaagaggcc ttactaacat gtaggccatg gtaaggcgtt tctgtgtttt gttttgcttt | 120 |
| gtctttatcc taaaatgcca ttggcaagtt tattgcag | 158 |

SEQ ID NO: 26

| | |
|---|---|
| gtgagtcgcc cccttcctct gctccgagag ggaaatggga attagggtgg tggcagagcc | 60 |
| caaagaggcc ttgtaggcca tggtaaggca tttctatgtt ttattttact ttgtctttat | 120 |
| cctaaaatgc ccttggcaag tttcttgcag | 150 |

SEQ ID NO: 27

| | |
|---|---|
| gtgagtcgcc cccttcctct gctccgagag ggaaatggga attagggtgg tggcagagcc | 60 |
| caaagaggcc ttgtaggcca tggtaaggca tttctatgtt ttattttact ttgtctttat | 120 |
| cctaaaatgc catgggcgag tttattgcag | 150 |

SEQ ID NO: 28

| | |
|---|---|
| gtgagtcgcc cccttcctct gctccgagag ggaaatggga attagggtgg tggcagagcc | 60 |
| caaagaggcc ttgtaggcca tggtaaggca tttctatgtt ttattttact ttgtctttat | 120 |
| cctaaaatgc catttgcaag tttcttgcag | 150 |

SEQ ID NO: 29

| | |
|---|---|
| catttctatg ttttatttta ctttgtcttt atcct | 35 |

SEQ ID NO: 30//mCardinal-P2A cloning forward primer:
CTTTCGACCTGCAGCCCAAGGCCGCCATGGTGAGCAAGGGCGAG
SEQ ID NO: 31//mCardinal-P2A cloning reverse primer:
CCAGCCTGCTTCAGCAGAGAGAAGTTGGTGGCTCCGCTTCCCTTGTACAGCTC
GTCCATG
SEQ ID NO: 32//P2A-mEmerald-synMAP3K7i250 cloning forward primer:
CTGCTGAAGCAGGCTGGTGACGTGGAGGAGAATCCCGGCCCTATGGTGAGCA
AGGGCGAG
SEQ ID NO: 33//P2A-mEmerald-synMAP3K7i250 cloning reverse primer:
CAAATTTTGTAATCCAGAGGTTGATTACTTGTACAGCTCGTCCATG
SEQ ID NO: 34//GPATCH8 sgRNA #1:
ATGCTGAAGATGCTACCGAA
SEQ ID NO: 35//GPATCH8 sgRNA #2:
CATGTTCAAACCAACCACAG
SEQ ID NO: 36//GPATCH8 sgRNA #3:
GGTCTGAGACAGAAGACACA
SEQ ID NO: 37//SUGP1 sgRNA #1:
CTATGAGAAGGGGAAGCCTG
SEQ ID NO: 38//SUGP1 sgRNA #2:
AACTTTTCCAGCTCGTCTGG
SEQ ID NO: 39//Renilla sgRNA:
GGAACACGGCCGTATTAGGG
SEQ ID NO: 40//Gpatch8 shRNA #1:
TGCTGTTGACAGTGAGCGATACAAGGATTATGTTGACAAATAGTGAAGCCAC
AGATGTATTTGTCAACATAATCCTTGTACTGCCTACTGCCTCGGA
SEQ ID NO: 41//Gpatch8 shRNA #2:
TGCTGTTGACAGTGAGCGATCCTACAGTTGTAATCCTTTATAGTGAAGCCACA
GATGTATAAAGGATTACAACTGTAGGAGTGCCTACTGCCTCGGA
SEQ ID NO: 42//NTC shRNA:
TGCTGTTGACAGTGAGCGCTTAAATAACTACTGACGTCCGTAGTGAAGCCAC
AGATGTACGGACGTCAGTAGTTATTTAATTGCCTACTGCCTCGGA
SEQ ID NO: 43//GPATCH8 (human) forward primer:
tggaagctgggccagggatt
SEQ ID NO: 44//GPATCH8 (human) reverse primer:
gcgccgttcggtagcatctt
SEQ ID NO: 45//Gpatch8 (murine) forward primer:
TGGAAGCTGGGCCAGGGATT
SEQ ID NO: 46//Gpatch8 (murine) reverse primer:
GACGCCGTTCGGTAGCATCC
SEQ ID NO: 47//SUGP1 forward primer:
GTGCAGAGGGACGTGGATGC
SEQ ID NO: 48//SUGP1 reverse primer:
GCCCACTAGACCCACAGGCT
SEQ ID NO: 49//GAPDH (human) forward primer:
CTTTTGCGTCGCCAGCCGAG
SEQ ID NO: 50//GAPDH (human) reverse primer:
CCAGGCGCCCAATACGACCA
SEQ ID NO: 51//Gapdh (murine) forward primer:
AGGTCGGTGTGAACGGATTTG
SEQ ID NO: 52//Gapdh (murine) reverse primer:
TGTAGACCATGTAGTTGAGGTCA
SEQ ID NO: 53//mEmerald forward primer:
CACATGAAGCAGCACGACTTC
SEQ ID NO: 54//mEmerald reverse primer:
GTCCTCCTTGAAGTCGATG
SEQ ID NO: 55//MAP3K7 (human) forward primer:
TGTCTTGTGATGGAATATGCTG
SEQ ID NO: 56//MAP3K7 (human) reverse primer:
TCCCTGTGAATTAGCGCTTT
SEQ ID NO: 57//Map3k7 (murine) forward primer:
GTTGTCACGTGTGAACCATC
SEQ ID NO: 58//Map3k7 (murine) reverse primer:
CATGAGCAGCAGTGTAGTAAG
SEQ ID NO: 59//SMURF2 forward primer:
AGCCCTGGCAGACCTCTTAGCT
SEQ ID NO: 60//SMURF2 reverse primer:
ACCTGGCCTTGTTGCGTTGTCC
SEQ ID NO: 61//PPM1M forward primer:
TGTCCAACGAGCAGGTGGCATG
SEQ ID NO: 62//PPM1M reverse primer:
CTTGGCCCTGACTGTGCAAGGG
SEQ ID NO: 63//ZDHHC16 forward primer:
CAGACCCCACCACCCACCTTCT
SEQ ID NO: 64//ZDHHC16 reverse primer:
GCCTGTAGCCGACGTCTCTCCT
SEQ ID NO: 65//sgRNA for human SF3B1 K700E mutation:
UGGAUGAGCAGCAGAAAGUU
SEQ ID NO: 66//sgRNA for editing AAVS1:
GGGCCACUAGGGACAGGAU
SEQ ID NO: 67//sgRNA for GPATCH8 knockout in primary human cells:
ATGCTGAAGATGCTACCGAA
SEQ ID NO: 68//sgRNA to insert 3X-FLAG:
GAGGAGUGAAGGCGGCAAAA
SEQ ID NO: 69//donor DNA sequence for inserting 3S-FLAG:
GGCGACGCCCGTGTTTACCTGAAAGTCTCGGTCTTCGTTGAAGCGGGAGAAG
CGGTCCGCTCCGGAGCTGCCCTTGTCGTCGTCGTCCTTGTAGTCGATGTCGTG
GTCCTTGTAGTCACCGTCGTGGTCCTTGTAGTCCATGTTGCCGCCTTCACTCCT
CTCAGGACGACGCTCTCCGGTTCGCTCCTTCCCTCCTGC
SEQ ID NO: 70//PCR and sequencing primer:
GGGAACCGGGGAGGGGTGGGTG (forward)
SEQ ID NO: 71//PCR and sequence primer:
AGAGGGCTGGGAGCCGGAGATGA (reverse)

1. A method of treating a subject with cancer, wherein the cancer is characterized by a change-of-function or loss-of-function mutation in a recurrently mutated RNA splicing factor gene, the method comprising:
administering to a subject a therapeutically effective amount of a composition that inhibits;
(i) a trans-acting splicing factor; and/or
(ii) inhibits expression of a cis-acting splicing factor,
wherein a cancer-causing error triggered by the change-of-function or loss-of-function mutation in a recurrently mutated RNA splicing factor gene is partially or fully rescued by inhibiting expression of the trans-acting splicing factor and/or inhibiting expression of the cis-acting splicing factor.

2. (canceled)

3. The method of claim 1, wherein the recurrently mutated RNA splicing factor gene comprises SF3B1, and wherein the recurrent change-of-function mutation in SF3B1 results in an amino acid substitution comprising E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, I704F, G740E, G742D, A762V, Y765C, D781E, D781G, M784I, E802Q, M971T, M971V, or combinations thereof, with reference to the wild-type amino acid sequence set forth in SEQ ID NO: 1.

4. (canceled)

5. The method of claim 1, wherein the cancer is selected from a myelodysplastic syndromes (MDS), a chronic myelomonocytic leukemia (CMML), a chronic lymphocytic leukemia (CLL), an acute myeloid leukemia (AML), an uveal melanoma, a mucosal melanoma, a skin melanoma, a breast cancer, a pancreatic cancer, an endometrial cancer, a liver cancer, a lung cancer, a mesothelioma, or other cancers with recurrent SF3B1 mutations.

6. The method of claim 1, wherein the composition comprises:
(i) a nucleic acid construct that inhibits expression of the trans-acting splicing factor and/or inhibits expression of the cis-acting splicing factor by attenuating the expression of a target gene, wherein the trans-acting splicing factor and/or the cis-acting splicing factor are encoded by the target gene; or
(ii) at least one protein-binding moiety that binds to at least one protein of interest and at least one tag that promotes degradation of the at least one protein of interest, wherein the at least one protein of interest comprises the trans-acting splicing factor and/or the cis-acting splicing factor.

7. (canceled)

8. The method of claim 1, wherein the composition comprises a small molecule that disrupts a protein-protein interaction required for the trans-acting splicing factor and/or the cis-acting splicing factor to recognize and bind to its splice site.

9. The method of claim 8, wherein the small molecule binds to:
(i) the trans-acting splicing factor and/or the cis-acting splicing factor blocking binding of the trans-acting splicing factor and/or the cis-acting splicing factor to at least one facilitating protein, wherein the trans-acting splicing factor and/or the cis-acting splicing factor cannot bind to its splice site without first binding to the facilitating protein; or
(ii) at least one facilitating protein blocking binding of the at least one facilitating protein to the trans-acting splicing factor and/or the cis-acting splicing factor, wherein the trans-acting splicing factor and/or the cis-acting splicing factor cannot bind to its splice site without first binding to the facilitating protein.

10. (canceled)

11. The method of claim 1, wherein the trans-acting splicing factor comprises a protein encoded by a GPATCH8 gene, and
wherein the protein encoded by the GPATCH8 gene is a G patch domain-containing protein 8 (GPATCH8) protein.

12. (canceled)

13. The method of claim 11, wherein the therapeutically effective amount of the composition inhibits expression or function of the GPATCH8 protein by at least 25%, wherein the GPATCH8 protein cannot bind to its 3′ recognition splice site if GPATCH8 protein expression or function is reduced by at least 25%, thereby partially or fully rescuing mis-splicing caused by the mutation in a recurrently mutated RNA splicing factor gene.

14. (canceled)

15. The method of claim 1, wherein the composition further comprises a therapeutic agent.

16. An in vitro method to screen for a trans-acting splicing factor and/or a cis-acting splicing factor required for mis-splicing in a change-of-function or loss-of-function mutation in a recurrently mutated RNA splicing factor gene, the method comprising:
(a) generating an expression cassette comprising a coding sequence (CDS) interrupted by at least one artificial nucleic acid intron, a promoter operatively linked to the CDS, wherein upon splicing of the artificial nucleic acid intron the CDS encodes a detectable reporter protein in wild-type cells and wherein the CDS does not encode a detectable reporter protein in cells comprising the recurrently mutated RNA splicing factor gene;
(b) contacting the construct generated in (a) to a population of cells;
(c) performing a positive enrichment CRISPR screen to identify at least one gene whose knockout enhances expression of the detectable reporter protein in cells comprising the recurrently mutated RNA splicing factor gene; and
(d) detecting expression of the detectable reporter protein, wherein expression of the detectable reporter protein in cells comprising the recurrently mutated RNA splicing factor gene is increased if the gene knockout is required for mis-splicing in recurrently mutated RNA splicing factor gene.

17. The method of claim 16, wherein the artificial nucleic acid intron comprises one or more features selected from:
(i) a 5′ splice site;
(ii) a canonical 3′ splice site;
(iii) at least one cryptic 3′ splice site, that is within about 100 nucleotides upstream of the canonical 3′ splice site or within about 50 nucleotides downstream of the canonical 3′ splice site;
(iv) a pyrimidine-rich domain comprising at least 6 consecutive nucleotides, wherein the sequence of the pyrimidine-rich domain is at least 60% pyrimidine nucleotides, and wherein the pyrimidine-rich domain is within at least 50 nucleotides of a cryptic 3′ splice site; and
(v) at least one branchpoint at least 15 nucleotides upstream of the canonical 3′ splice site.

18. The method of claim 17, wherein the artificial nucleic acid intron is at least about 50 nucleotides to about 1000 nucleotides in length, and wherein the artificial nucleic acid intron is derived from a human wildtype intron comprising intron 1 of MTERFD3, intron 4 of MYO15B, intron 10 of SYTL1, intron 11 of SYTL1, intron 4 of MAP3K7, intron 1 of ORAI2, or intron 1 of TMEM14C.

19. (canceled)

20. The method of claim 17, wherein the human wildtype intron from which the artificial nucleic acid intron is derived is one of the following:
intron 1 of MTERFD3 comprising a sequence set forth in SEQ ID NO:2;
intron 4 of MYO15B comprising a sequence set forth in SEQ ID NO:3;
intron 10 of SYTL1 comprising a sequence set forth in SEQ ID NO:4;
intron 11 of SYTL1 comprising a sequence set forth in SEQ ID NO:5;
intron 4 of MAP3K7 comprising a sequence set forth in SEQ ID NO:6;
intron 1 of ORAI2 comprising a sequence set forth in SEQ ID NO:7; or
intron 1 of TMEM14C comprising a sequence set forth in SEQ ID NO:8.

21. The method of claim 20, wherein the artificial nucleic acid intron is derived from a human wildtype intron 4 of MAP3K7, and wherein the intron further comprises one, two, three, or more of the following features:
(i) a 5′ splice site comprising a GT dinucleotide immediately followed by a consensus 5′ splice site context, optionally wherein the consensus 5′ splice site context comprises AAG, GAG, or GTG;
(ii) a canonical 3′ splice site comprising an AG dinucleotide immediately preceded by a C or T;
(iii) at least one cryptic 3′ splice site, located at least 5 nucleotides upstream of the canonical 3′ splice site, with an AG dinucleotide and comprising a sequence that is a weaker 3′ splice site than is the canonical 3′ splice site, where splice site strength is estimated with the MaxEntScan algorithm or similar methods;
(iv) a pyrimidine-rich domain comprising at least 15 consecutive nucleotides, wherein the sequence of the pyrimidine-rich domain is at least 60% pyrimidine nucleotides and at least 40% thymine nucleotides, and wherein the pyrimidine-rich domain is within at least 30 nucleotides of a cryptic 3′ splice site; and
(v) at least one branchpoint at least 20 nucleotides upstream of the canonical 3′ splice site.

22. The method of claim 18, wherein the artificial nucleic acid intron has:
(i) a 5′ end domain with about 10 to about 150 nucleotides having at least 50% sequence identity to a sequence of the 5′-most 10 to about 150 nucleotides of the wildtype intron, and/or
(ii) a 3′ end domain with about 50 to about 350 nucleotides having at least 50% sequence identity to a sequence of the 3′-most 50 to about 350 nucleotides of the wildtype intron.

23. (canceled)

24. The method of claim 20, wherein the artificial nucleic acid intron has a sequence with at least 75% sequence identity to a sequence selected from SEQ ID NOS: 9-28.

25. The method of claim 17, wherein:
(i) the 5′ splice site comprises a sequence selected from GTGAG, GTAAG, GTGCG, GTACG, GTGGG, GTAGG, GTGTG, GTATG, or GTATC;
(ii) the canonical 3′ splice site comprises a sequence selected from AAG, CAG, or TAG;
(iii) the at least one cryptic 3′ splice site comprises a sequence selected from AAG, CAG, GAG, TAG, ATG, CTG, GTG, or TTG; and
(iv) the pyrimidine-rich domain is characterized by one, two, three, or all of the following:
(a) wherein the pyrimidine-rich domain comprises at least 15 consecutive nucleotides;
(b) wherein the pyrimidine-rich domain has a sequence with at least 60% pyrimidine nucleotides and is at least 40% thymine nucleotides;
(c) wherein the pyrimidine-rich domain is within at least 30 nucleotides of a cryptic 3′ splice site; or
(d) wherein the pyrimidine-rich domain has a sequence with at least 50% sequence identity to any 20 nucleotides selected from the sequence set forth as SEQ ID NO:29; and
(v) the at least one branchpoint is at least 20 nucleotides upstream of the canonical 3′ splice site, wherein the branchpoint nucleotide is an adenine, and wherein the branchpoint and surrounding sequence context has sequence identity of at least 60% to the sequence tactaAca, wherein the uppercase A is the branchpoint nucleotide.

26-27. (canceled)

28. The method of claim 17, wherein the artificial nucleic acid intron comprises a plurality of cryptic 3′ splice sites within about 100 nucleotides upstream of the canonical 3′ splice site or within about 100 nucleotides downstream of the canonical 3′ splice site, and wherein each of the plurality of the cryptic 3′ splice sites comprises a sequence independently selected from AAG, CAG, GAG, TAG, ATG, CTG, GTG, or TTG.

29-31. (canceled)

32. The method of claim 16, wherein the artificial nucleic acid intron is configured to be spliced differently in a cancer cell comprising a change-of-function or loss-of-function mutation in a recurrently mutated RNA splicing factor gene relative to the splicing pattern of the intron in a cell lacking a change-of-function or loss-of-function mutation in a recurrently mutated RNA splicing factor gene.

33. The method of claim 16, wherein the RNA splicing factor gene is SF3B1, and wherein the recurrent change-of-function mutation in SF3B1 results in an amino acid substitution selected from E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, 1704F, G740E, G742D, A762V, Y765C, D781E, D781G, M784I, E802Q, M971T, M971V, or combinations thereof, with reference to the wild-type amino acid sequence set forth in SEQ ID NO:1.

34. (canceled)

35. The method of claim 17, wherein the expression cassette further comprises a first exon domain and a second exon domain, wherein the artificial nucleic acid intron is disposed between the first exon domain and the second exon domain, and
wherein the combination of the first exon domain and the second exon domain without the artificial nucleic acid intron encodes part or all of a protein of interest.

36-37. (canceled)

38. The method of claim 16, wherein;
(i) detecting expression of the detectable reporter protein comprises quantifying the amount of the reporter protein; and/or
(ii) the reporter protein comprises a fluorescent or luminescent protein.

39. (canceled)


SEQUENCE LISTING (81 sequences)

SEQ ID NO: 1 - protein, 1304 aa, Homo sapiens
MAKIAKTHEDIEAQIREIQGKKAALDEAQGVGLDSTGYYDQEIYGGSDSRFAGYVTSIAATELEDDDDDYSSSTSLLGQKKPGYHAPVALLNDIPQSTEQYDPFAEHRPPKIADREDEYKKHRRTMIISPERLDPFADGGKTPDPKMNARTYMDVMREQHLTKEEREIRQQLAEKAKAGELKVVNGAAASQPPSKRKRRWDQTADQTPGATPKKLSSWDQAETPGHTPSLRWDETPGRAKGSETPGATPGSKIWDPTPSHTPAGAATPGRGDTPGHATPGHGGATSSARKNRWDETPKTERDTPGHGSGWAETPRTDRGGDSIGETPTPGASKRKSRWDETPASQMGGSTPVLTPGKTPIGTPAMNMATPTPGHIMSMTPEQLQAWRWEREIDERNRPLSDEELDAMFPEGYKVLPPPAGYVPIRTPARKLTATPTPLGGMTGFHMQTEDRTMKSVNDQPSGNLPFLKPDDIQYFDKLLVDVDESTLSPEEQKERKIMKLLLKIKNGTPPMRKAALRQITDKAREFGAGPLFNQILPLLMSPTLEDQERHLLVKVIDRILYKLDDLVRPYVHKILVVIEPLLIDEDYYARVEGREIISNLAKAAGLATMISTMRPDIDNMDEYVRNTTARAFAVVASALGIPSLLPFLKAVCKSKKSWQARHTGIKIVQQIAILMGCAILPHLRSLVEIIEHGLVDEQQKVRTISALAIAALAEAATPYGIESFDSVLKPLWKGIRQHRGKGLAAFLKAIGYLIPLMDAEYANYYTREVMLILIREFQSPDEEMKKIVLKVVKQCCGTDGVEANYIKTEILPPFFKHFWQHRMALDRRNYRQLVDTTVELANKVGAAEIISRIVDDLKDEAEQYRKMVMETIEKIMGNLGAADIDHKLEEQLIDGILYAFQEQTTEDSVMLNGFGTVVNALGKRVKPYLPQICGTVLWRLNNKSAKVRQQAADLISRTAVVMKTCQEEKLMGHLGVVLYEYLGEEYPEVLGSILGALKAIVNVIGMHKMTPPIKDLLPRLTPILKNRHEKVQENCIDLVGRIADRGAEYVSAREWMRICFELLELLKAHKKAIRRATVNTFGYIAKAIGPHDVLATLLNNLKVQERQNRVCTTVAIAIVAETCSPFTVLPALMNEYRVPELNVQNGVLKSLSFLFEYIGEMGKDYIYAVTPLLEDALMDRDLVHRQTASAVVQHMSLGVYGFGCEDSLNHLLNYVWPNVFETSPHVIQAVMGALEGLRVAIGPCRMLQYCLQGLFHPARKVRDVYWKIYNSIYIGSQDALIAHYPRIYNDDKNTYIRYELDYIL

SEQ ID NO: 2 - DNA, 1753 bp, Homo sapiens
  source 1..1753: mol_type = other DNA
gtgagtcgcccccttcctctgctcctgggcgtgttcctcaccagcggcgccgcagcggtcagggcccgcaaaaccccacgcctcgccagacgctcagctcagcctgccgggtttctctggaaaagcggaggccccacagtgaatgtgcggccttttgatcggtcctggagatcttccgtggaccctcgtttttccttgcttataaacgttggtcaatttgaaactggcagcagggcatatgtgttacgaaagaagaactttattgaaaccagtggtggtgtagagaagaccatactaaccataactgtgtattacagaagtgaatttcactccccaaaaatgcttaagaaacaagcatcctagaacagcccacattgtgatttaacaaacatttaccaaaagcattgctatatgtgggggcacagggctaagagcaggcaggactctggagccaacctgtggggtttgagtccaaattctgccggttttttgttttttgtttttttttttagctgagtaaccttgggcaagttcctttacctgcgtctgtttcctcattttaaaatgagttgttgtgatgatttaatgatttagtatatgtgaagagttttctgcatattctaagtgatttatggtattaactcgtttgatctttgcagtcacccaggagatagtatcaccattttcctgatgccaaactgaggctcagagaggtgaagtaacttcttcaagtcacaacacaactgttgtccaggaatcaggcagtctggctctgctaacccagttttacccactgcattaatgtaccgcatccacttgttcactcagtttcacctgacaaatactaattttgtctctctgtggaacaagttctttgctgggtgcagggaataaagcagaacaccgttcagttcctgccttcccaacgcttgtaaatctagtgcaggtcattcatggccgggctgcagggtgtccatgaatcctgctaaaatcatttgcgaaatgttgtgagcgtgggtcttttcttctggaaagagagtctgtgactctcaggttctgagttgatctgtgatccaaacagaggtgtgaagaagtgttgttttagcagatctgtgcctagtatgcctgatacacagcttgaacttcaaacaaacagcaacaaaattcctactggttggaagaatgaagaacttgtaagagcataataagtgctgcagtagcgtccggagcagggtgctgtggaagagcacacagaaggccacctgcccaggaatgcctcctagaggcagtgaaggcagctgggtgaaggatagggaagcagttcattggaacactgtggtgaccagtctgtccatgtagtgttgagacagaaacaacacgtgaacagctgcagctgttgtaccacttgtgcgcaagtgtaaaagtccatggtgccatgaaatcatgtaatggggacaggaggcttcatgtattcgtgaaggttagaaaagtttccttgagaaagtgacacttaggcctaaaagttggagcattttgggcatggaatctggcccacagtcttcaaagaccacatggtagaaaccagtagagcaccagggactgacaggaagcaaatgcagctggtgcaggagagggaaatgggaattagggtggtggcagagcccaaagaggccttgtaggccatggtaaggcatttctatgttttattttactttgtctttatcctaaaatgccattggcaagtttattgcag

SEQ ID NO: 3 - DNA, 369 bp, Homo sapiens
  source 1..369: mol_type = other DNA
gtgagtggtggccttcctgggtggctgaacccatgggccacactgagggttgatggggcggggcctgaggtcttcactggaagccactggtatgctccactgctgggctacagttctggactcctcagggccgagctcctggctgaaatcatcacatgagcccctggaatgtggcatcttggggactttttcttttctgtcgggtctctccatgccctgggactatctttccacatttatgggggtctctccaggctagctgtcacttctgggtggcacatcccacttcctgggttcactggggtggtcctatgtgcccctccctggagaccctaccaaccaacacatccttagctcctcccactgcag

SEQ ID NO: 4 - DNA, 308 bp, Homo sapiens
  source 1..308: mol_type = other DNA
gtgaggctgtgaccacgatgcggttccccgttaatgaactggacgcccccttcctgcggggctaggtggcaagggcagccagtaacgtcattgcccggaggatcggcggagggggcccattaactcgttatccagtgttgtcagcctcgtggtgggcgtggtgatagtgcaggtccccattaatgcccttaggggctccccagaattccatcatggtaggaacgcggtaggacctgcctcagccaactcgcgcagcatctacgcgggccaccagcagtgctccactaaagctcacctcctgtcctcag

SEQ ID NO: 5 - DNA, 305 bp, Homo sapiens
  source 1..305: mol_type = other DNA
gtgaggcagccaggccgcgtggggagacctgcggcccgggtctcctgcatttaccccaccaggctctcccgcagccccctcacaccccgccttcgacagaacctcccctcaacctcttaacctcatggccccaggcgaagcccggccggccacggccccttccccgagggcgctaggacccctaggttctgcccctgcaggccccgccgtctcttctagccgcaccccatccgggtctgcagaccccaccctcctgaggcccctttccattagcccctgctccacgataagcccgcctctcgcag

SEQ ID NO: 6 - DNA, 1407 bp, Homo sapiens
  source 1..1407: mol_type = other DNA
gtgagtgtcattagacctgtctttatctagtggattaaaataatttgaaaaattttaatataaaccctaagttgtttaacattcttcacaatttgccgtcagagtcccaaaagggcataatttttaaaaatctgctaagaataaaaatggaaagataagattctataacattagtatgtgaaatttagaggtctatcaattttttaagtagatggtactttgcatttcattaaaagcatttaagtaaaagtttactttttaaatagattttaaaatatcttcagatttttttctttttgccctctcataactttgagcctcctcattttagaattctttaaccatatttcgatttcactctgctttattttttcttttttttcaaaaaaaaatgtgaaaactcacttcacacaaccttttatttgtagtaatgaaaaaaaactttgtgtagttacattggagaacagagttcttttaccctaaaaataccaaccatatgcagaaatgtattgtctcttgtagcaccagttatataaaacaaattgttacattttattttttaattagtattattttatgtaaacttttacttatatttactgcccatgtgttcatcattgtgatggatatctaataccccattgtcttatttgattgaactacttctggtttttagccattacagaaaatacaggtgcaaacatcttgatatacattgttttgtttctttctgtactgttctctgcctctctcttgtagtagtgtgccataagactgctcataatttatattactttataaaagtctttatctgtttgaaaaaatgtggtaatgtatattgattttacttcaatgtaggaataacatttaatagatgtactattaaatgttaatatcatggcaattatccactcaaaattagtagatattttcatcctttttatttagaagaaaaattaaaaggccaggcatggtggctcactcctgtaatcccagcactttgggaggccaaggcaggcagatcatgaggtcaagagatcaagaccatcctggccaacatggtgataccctatctctactaaaaatacaaaaattagctgggcgtggtggcacacgcctgtagtcccagctactcgggaggctgagccaggagaatcgcttgaacccaggaggtggagtttacagtgagccaagatcgcaccactgcactccagcctggcgacaaagtgagagtccgtctcaaaaaacaaaaagaaaaaagaaagtaaaaaattaaaagcaggactgatggagcatatgtatatgtagaaaaatacccagtcatgtgttctgatagtgcttgcattcacattgtgtcttttgttatatgcaataaagttctttttagtttgtgcctttctttcgcag

SEQ ID NO: 7 - DNA, 2563 bp, Homo sapiens
  source 1..2563: mol_type = other DNA
gtgagtgtggcggcgcgggggcctggcgcggggagcggggtccctgcggctgggggggccggaattggaccggggacgctcggcggggaaggggacgactgtaggctgcccgggcgcggtcactacccatccggggttggtgacctccaccccgcgggatccctggcggggtgcggcgcccagaggatcagggtcggcggcgcagccggttccggacaccggggcgtccctgggggagggacagggatgcagcctagcgcccctgggaggccccctcccagtgcgcctgctggagagagacactcgccattgccggtgccccagatgcttagaggaagtcaaactccaagaccgccctctggaaatgtgtccagtaggccagccaggaagaaaagctcatctctagagaggggatcgcgttattctacaagacctgttttctcggactgctcattaaagcaaaaaaaaaaaaaaaaaaaaaaaagcaaccccacaaggttcagtcccggagcatcctctagggtctgtgccacctgacagaagtacagggctccatcctgccccaccccaccccctgcctgggatccagtgtgaggcccagactctgtccctccccctgggccaccgggcctggcacacacagttggcattgggaacataaaggatggatttgaaccccctcctgggctgctccttcgagacctggagacctgtctcaagacccagctccaggcagacagacatcgtttcctccacaatcccacccactctggggacatctgcagggcctcaccgatggggcatgggaggcctggcggctcccaggaagccaccctcagccctcactgccctgagctcttcacctccactctcagacctgagcccagcccacccttactgatggggcatccctcttgttcacccttccttcctttggaggccaccaggctctcccttgcgtcccactcctgccagtcggccagccatttccctgggttgaactgtggtcccaaaccagaatgatctagcagtgcccagaccgttccactcaccaatttcttccattctcctgtggctggacgcccagtcatgctgcccagaccctctggctcattccacagccccaggctccaagtgtcttccacccacatctccctgccccctccaccccccaggtggcagccagtcttccctccatatctcctggacaaggagagccaccccacatgtacacctggagtggccttcccgtctctcggaatccttccatgcaggcagcatcttgtctttgaggaaaggtccctcttttttaaaattctttttagaggccaggcgcagtggttcatgcctataatctcagcactttgggaggccaaggtgggcggatcacttgagctcaggagttctagaccagcctggccaacatagcaaaaccctgtctctaccaaaaataccaaaaattagccaggtatggtggcacacacatgtagtcccagctactcgggtggttgaggcgggaggatcgcttgagcctgggaggtagaatttgcagggagctgagattgtgccactgcactccagccttggtaacacagcaggaccccatgtcaaaaaaaattatttttagagacagcgtcttactctgttgcctaggctggtctccagctcctggccgcaggtgatcctcccaccttggcctcacaaaacactgggattacaggcatgaggcactttggcatgcaaagggccgctctgtcccctccccggtgttacctgcaagcaccttttcctggccttggtgtgaggcagccagtcctctgctccctgccctccattgaagcctttagctcagtggatgaggattgcccccccctcttttttttttttttttttgagatggagtctcgctctgttgcccaggctggagtgcagtggtgtgctcttggctcactgcaacctctgccaccacacccagctatttttttttttttaagacagagtctcactgtgtcacccaggctggagtgccgcggtaggatctcagctcactgcaacctccgcctcctgggatcaggcaattctcctgcctcagcctcctgagtagctgggattacaggcgcccaccaccatgcccagttactttttgtatttttagaagagacaggtttccaccatgttgtccaggctagtctcgaactcctgacctctagtgatccgcccacctcagcctcccaaagatgacaggtgtgagctactgctcctgacccacatccccttaccttgctcccctactgggaggccatgaattctggcctctttcatcttggatctttaagtaccaagtgcgctaagcacctgtgtgatggaggagggaggacggagggagcaaaggttagcgtctcacccctccagtctctgtatactgcggtcatcagacctgcatgctcaattctgttaactatgggggtggggggagaggaaaaatgcagtttccaaattacagcattttctattatgtggagctaacctacggattgcagcgtttctctgaattctccccaag

SEQ ID NO: 8 - DNA, 1328 bp, Homo sapiens
  source 1..1328: mol_type = other DNA
gtgcgagtatttggggattattcttattttctgccacttttaacttttagggattatttaggagtttcacggccgtctgcttttcgtccccccgattcagcgggccttcaggccgtgtggtcggtgcttttctcgttgggtattttctgcttttaaaagaagttgtggatgcgcggagcccctgggctcctgaggcaaaggcttacccatgtagcatagtgttgcctcgtttcttggtgattttcttgctccatctctttaaaagcctcccttactcggtgccgtctcgagttaagaactgtgggcaagatcccaagcccgctgcccttccctgttttatggaaacttaattctttttttttttttttttgtccttgagggagggacttgctctgtcgcccatgcttgagtgcagttgcctgaatatgtctcattgcagcctctgcctcctgggctcaagccatcctcccacttcagcctcccaagtagctgggatttacagtcgcatatcaccatgcgcgattaatttttttatattttttagtagacaatgggtttcactatgttggccaggctggtcttgaactcctaaccccaggtgatccgcccgcctcggcctcctaaagtactgggatgacaggcgtcagccactgcgcttggcctaattctttaaacagaataaacggggtatgcattgctttcatcttttggctcactggacacaggatactttctgtaagaaaatagaagctgtttttccaagggtgtagtgtcacatgtgaatatgaccactgtttccgtatattttatcctctcctactactgccctcctaacaagaactgtgagttggacgcagaagtttctaaaaaagttgagctttgaaattggctgttgcagcagggatgaaaagcaacacccctacctcccctcaaaagagacattaaagtagttggattaagggcacgggagtatttgcttttgaatttagtgataacatgggtagctgatgaaatgactaacacattccctgattttagagctggtcagtggatcttgctgagtttcccgtgggcctatgtgattaaaactgaggttttcatgacaatggtcagcatctgttcagggtttactaggtgctaagcacctttacatgtgacatccattgaatgctcactacagccccaggaaatcggtaccagtgttatcctcatggtacagtgaagaatactgagacttaggttgcgtagcttgcaggttggacacacttctttctgactgctggagagctgtgcttttaactacctctgatccagcttgttttctgcag

SEQ ID NO: 9 - DNA, 250 bp, synthetic construct
  source 1..250: mol_type = other DNA
gtgagtcgcccccttcctctgctcctgggcgtgttcctcaccagcggcgccgcagcggtcagggcccgcaaaaccccacgcctcgccagacgctcagctccaggaagcaaatgcagctggtgcaggagagggaaatgggaattagggtggtggcagagcccaaagaggccttgtaggccatggtaaggcatttctatgttttattttactttgtctttatcctaaaatgccattggcaagtttattgcag

SEQ ID NO: 10 - DNA, 150 bp, synthetic construct
  source 1..150: mol_type = other DNA
gtgagtcgcccccttcctctgctccgagagggaaatgggaattagggtggtggcagagcccaaagaggccttgtaggccatggtaaggcatttctatgttttattttactttgtctttatcctaaaatgccattggcaagtttattgcag

SEQ ID NO: 11 - DNA, 100 bp, synthetic construct
  source 1..100: mol_type = other DNA
gtgagtcgcccccttaggccttgtaggccatggtaaggcatttctatgttttattttactttgtctttatcctaaaatgccattggcaagtttattgcag

SEQ ID NO: 12 - DNA, 350 bp, synthetic construct
  source 1..350: mol_type = other DNA
gtgagtggtggccttcctgggtggctgaacccatgggccacactgagggtgtcttcactggaagccactggtatgctccactgctgggctacagttctggactcctcagggccgagctcctggctgaaatcatcacatgagcccctggaatgtggcatcttggggactttttcttttctgtcgggtctctccatgccctgggactatctttccacatttatgggggtctctccaggctagctgtcacttctgggtggcacatcccacttcctgggttcactggggtggtcctatgtgcccctccctggagaccctaccaaccaacacatccttagctcctcccactgcag

SEQ ID NO: 13 - DNA, 265 bp, synthetic construct
  source 1..265: mol_type = other DNA
gtgagtggtggccttcctgggtggctgaacccatgggccacactgagggttgatggggcggggcctgaggtcttcactggaagccactggtatgctccactctctccatgccctgggactatctttccacatttatgggggtctctccaggctagctgtcacttctgggtggcacatcccacttcctgggttcactggggtggtcctatgtgcccctccctggagaccctaccaaccaacacatccttagctcctcccactgcag

SEQ ID NO: 14 - DNA, 300 bp, synthetic construct
  source 1..300: mol_type = other DNA
gtgaggctgtgaccacgatgcggttccccgttaatgaactggacgcccccgggctaggtggcaagggcagccagtaacgtcattgcccggaggatcggcggagggggcccattaactcgttatccagtgttgtcagcctcgtggtgggcgtggtgatagtgcaggtccccattaatgcccttaggggctccccagaattccatcatggtaggaacgcggtaggacctgcctcagccaactcgcgcagcatctacgcgggccaccagcagtgctccactaaagctcacctcctgtcctcag

SEQ ID NO: 15 - DNA, 240 bp, synthetic construct
  source 1..240: mol_type = other DNA
gaccacgatgcggttccccgttaatgaactggacgcccccttcctgcggggctaggtggcaagggcagccagtaacgtcattgcccggagtggtgatagtgcaggtccccattaatgcccttaggggctccccagaattccatcatggtaggaacgcggtaggacctgcctcagccaactcgcgcagcatctacgcgggccaccagcagtgctccactaaagctcacctcctgtcctcag

SEQ ID NO: 16 - DNA, 300 bp, synthetic construct
  source 1..300: mol_type = other DNA
gtgaggcagccaggccgcgtggggagacctgcggcccgggtgcatttaccccaccaggctctcccgcagccccctcacaccccgccttcgacagaacctcccctcaacctcttaacctcatggccccaggcgaagcccggccggccacggccccttccccgagggcgctaggacccctaggttctgcccctgcaggccccgccgtctcttctagccgcaccccatccgggtctgcagaccccaccctcctgaggcccctttccattagcccctgctccacgataagcccgcctctcgcag

SEQ ID NO: 17 - DNA, 250 bp, synthetic construct
  source 1..250: mol_type = other DNA
gtgaggcagccaggcaggctctcccgcagccccctcacaccccgccttcgacagaacctcccctcaacctcttaacctcatggccccaggcgaagcccggccggccacggccccttccccgagggcgctaggacccctaggttctgcccctgcaggccccgccgtctcttcccatccgggtctgcagaccccaccctcctgaggcccctttccattagcccctgctccacgataagcccgcctctcgcag

SEQ ID NO: 18 - DNA, 250 bp, synthetic construct
  source 1..250: mol_type = other DNA
gtgagtgtcattagacctgtctttatctagtggattaaaataatttgaaaaattttaatataaaccctaagttgtttaacattcttcacaatttgccgtcaaagaaagtaaaaaattaaaagcaggactgatggagcatatgtatatgtagaaaaatacccagtcatgtgttctgatagtgcttgcattcacattgtgtcttttgttatatgcaataaagttctttttagtttgtgcctttctttcgcag

SEQ ID NO: 19 - DNA, 250 bp, synthetic construct
  source 1..250: mol_type = other DNA
gtgagtgtggcggcgcgggggcctggcgcggggagcggggtccctgcggctgggggggccggaattggaccggggacgctcggcggggaaggggacgactctctgtatactgcggtcatcagacctgcatgctcaattctgttaactatgggggtggggggagaggaaaaatgcagtttccaaattacagcattttctattatgtggagctaacctacggattgcagcgtttctctgaattctccccaag

SEQ ID NO: 20 - DNA, 250 bp, synthetic construct
  source 1..250: mol_type = other DNA
gtgcgagtatttggggattattcttattttctgccacttttaacttttagggattatttaggagtttcacggccgtctgcttttcgtccccccgattcagagccccaggaaatcggtaccagtgttatcctcatggtacagtgaagaatactgagacttaggttgcgtagcttgcaggttggacacacttctttctgactgctggagagctgtgcttttaactacctctgatccagcttgttttctgcag

SEQ ID NO: 21 - DNA, 150 bp, synthetic construct
  source 1..150: mol_type = other DNA
gtgagtcgcccccttcctctgctccgagagggaaatgggaattagggtggtggcagagcccaaagaggccttgtaggccatggtaaggcgtttctgtgttttgttttgctttgtctttatcctaaaatgccattggcaagtttattgcag

SEQ ID NO: 22 - DNA, 158 bp, synthetic construct
  source 1..158: mol_type = other DNA
gtgagtcgcccccttcctctgctccgagagggaaatgggaattagggtggtggcagagcccaaagaggccttgtaggccatggtaaggctactaacaatttctatgttttattttactttgtctttatcctaaaatgccattggcaagtttattgcag

SEQ ID NO: 23 - DNA, 158 bp, synthetic construct
  source 1..158: mol_type = other DNA
gtgagtcgcccccttcctctgctccgagagggaaatgggaattagggtggtggcagagcccaaagaggccttgtaggccatggtaaggcatttctattactaacagttttattttactttgtctttatcctaaaatgccattggcaagtttattgcag

SEQ ID NO: 24 - DNA, 158 bp, synthetic construct
  source 1..158: mol_type = other DNA
gtgagtcgcccccttcctctgctccgagagggaaatgggaattagggtggtggcagagcccaaagaggccttgtaggccatggtactaacataaggcgtttctgtgttttgttttgctttgtctttatcctaaaatgccattggcaagtttattgcag

SEQ ID NO: 25 - DNA, 158 bp, synthetic construct
  source 1..158: mol_type = other DNA
gtgagtcgcccccttcctctgctccgagagggaaatgggaattagggtggtggcagagcccaaagaggccttactaacatgtaggccatggtaaggcgtttctgtgttttgttttgctttgtctttatcctaaaatgccattggcaagtttattgcag

SEQ ID NO: 26 - DNA, 150 bp, synthetic construct
  source 1..150: mol_type = other DNA
gtgagtcgcccccttcctctgctccgagagggaaatgggaattagggtggtggcagagcccaaagaggccttgtaggccatggtaaggcatttctatgttttattttactttgtctttatcctaaaatgcccttggcaagtttcttgcag

SEQ ID NO: 27 - DNA, 150 bp, synthetic construct
  source 1..150: mol_type = other DNA
gtgagtcgcccccttcctctgctccgagagggaaatgggaattagggtggtggcagagcccaaagaggccttgtaggccatggtaaggcatttctatgttttattttactttgtctttatcctaaaatgccatgggcgagtttattgcag

SEQ ID NO: 28 - DNA, 150 bp, synthetic construct
  source 1..150: mol_type = other DNA
gtgagtcgcccccttcctctgctccgagagggaaatgggaattagggtggtggcagagcccaaagaggccttgtaggccatggtaaggcatttctatgttttattttactttgtctttatcctaaaatgccatttgcaagtttcttgcag

SEQ ID NO: 29 - DNA, 35 bp, synthetic construct
  source 1..35: mol_type = other DNA
catttctatgttttattttactttgtctttatcct

SEQ ID NO: 30 - DNA, 44 bp, synthetic construct
  source 1..44: mol_type = other DNA
ctttcgacctgcagcccaaggccgccatggtgagcaagggcgag

SEQ ID NO: 31 - DNA, 60 bp, synthetic construct
  source 1..60: mol_type = other DNA
ccagcctgcttcagcagagagaagttggtggctccgcttcccttgtacagctcgtccatg

SEQ ID NO: 32 - DNA, 60 bp, synthetic construct
  source 1..60: mol_type = other DNA
ctgctgaagcaggctggtgacgtggaggagaatcccggccctatggtgagcaagggcgag

SEQ ID NO: 33 - DNA, 46 bp, synthetic construct
  source 1..46: mol_type = other DNA
caaattttgtaatccagaggttgattacttgtacagctcgtccatg

SEQ ID NO: 34 - RNA, 20 nt, synthetic construct
  source 1..20: mol_type = other RNA
atgctgaagatgctaccgaa

SEQ ID NO: 35 - RNA, 20 nt, synthetic construct
  source 1..20: mol_type = other RNA
catgttcaaaccaaccacag

SEQ ID NO: 36 - RNA, 20 nt, synthetic construct
  source 1..20: mol_type = other RNA
ggtctgagacagaagacaca

SEQ ID NO: 37 - RNA, 20 nt, synthetic construct
  source 1..20: mol_type = other RNA
ctatgagaaggggaagcctg

SEQ ID NO: 38 - RNA, 20 nt, synthetic construct
  source 1..20: mol_type = other RNA
aacttttccagctcgtctgg

SEQ ID NO: 39 - RNA, 20 nt, synthetic construct
  source 1..20: mol_type = other RNA
ggaacacggccgtattaggg

SEQ ID NO: 40 - RNA, 97 nt, synthetic construct
  source 1..97: mol_type = other RNA
tgctgttgacagtgagcgatacaaggattatgttgacaaatagtgaagccacagatgtatttgtcaacataatccttgtactgcctactgcctcgga

SEQ ID NO: 41 - RNA, 97 nt, synthetic construct
  source 1..97: mol_type = other RNA
tgctgttgacagtgagcgatcctacagttgtaatcctttatagtgaagccacagatgtataaaggattacaactgtaggagtgcctactgcctcgga

SEQ ID NO: 42 - RNA, 97 nt, synthetic construct
  source 1..97: mol_type = other RNA
tgctgttgacagtgagcgcttaaataactactgacgtccgtagtgaagccacagatgtacggacgtcagtagttatttaattgcctactgcctcgga

SEQ ID NO: 43 - DNA, 20 bp, synthetic construct
  source 1..20: mol_type = other DNA
tggaagctgggccagggatt

SEQ ID NO: 44 - DNA, 20 bp, synthetic construct
  source 1..20: mol_type = other DNA
gcgccgttcggtagcatctt

SEQ ID NO: 45 - DNA, 20 bp, synthetic construct
  source 1..20: mol_type = other DNA
tggaagctgggccagggatt

SEQ ID NO: 46 - DNA, 20 bp, synthetic construct
  source 1..20: mol_type = other DNA
gacgccgttcggtagcatcc

SEQ ID NO: 47 - DNA, 20 bp, synthetic construct
  source 1..20: mol_type = other DNA
gtgcagagggacgtggatgc

SEQ ID NO: 48 - DNA, 20 bp, synthetic construct
  source 1..20: mol_type = other DNA
gcccactagacccacaggct

SEQ ID NO: 49 - DNA, 20 bp, synthetic construct
  source 1..20: mol_type = other DNA
cttttgcgtcgccagccgag

SEQ ID NO: 50 - DNA, 20 bp, synthetic construct
  source 1..20: mol_type = other DNA
ccaggcgcccaatacgacca

SEQ ID NO: 51 - DNA, 21 bp, synthetic construct
  source 1..21: mol_type = other DNA
aggtcggtgtgaacggatttg

SEQ ID NO: 52 - DNA, 23 bp, synthetic construct
  source 1..23: mol_type = other DNA
tgtagaccatgtagttgaggtca

SEQ ID NO: 53 - DNA, 21 bp, synthetic construct
  source 1..21: mol_type = other DNA
cacatgaagcagcacgacttc

SEQ ID NO: 54 - DNA, 19 bp, synthetic construct
  source 1..19: mol_type = other DNA
gtcctccttgaagtcgatg

SEQ ID NO: 55 - DNA, 22 bp, synthetic construct
  source 1..22: mol_type = other DNA
tgtcttgtgatggaatatgctg

SEQ ID NO: 56 - DNA, 20 bp, synthetic construct
  source 1..20: mol_type = other DNA
tccctgtgaattagcgcttt

SEQ ID NO: 57 - DNA, 20 bp, synthetic construct
  source 1..20: mol_type = other DNA
gttgtcacgtgtgaaccatc

SEQ ID NO: 58 - DNA, 21 bp, synthetic construct
  source 1..21: mol_type = other DNA
catgagcagcagtgtagtaag

SEQ ID NO: 59 - DNA, 22 bp, synthetic construct
  source 1..22: mol_type = other DNA
agccctggcagacctcttagct

SEQ ID NO: 60 - DNA, 22 bp, synthetic construct
  source 1..22: mol_type = other DNA
acctggccttgttgcgttgtcc

SEQ ID NO: 61 - DNA, 22 bp, synthetic construct
  source 1..22: mol_type = other DNA
tgtccaacgagcaggtggcatg

SEQ ID NO: 62 - DNA, 22 bp, synthetic construct
  source 1..22: mol_type = other DNA
cttggccctgactgtgcaaggg

SEQ ID NO: 63 - DNA, 22 bp, synthetic construct
  source 1..22: mol_type = other DNA
cagaccccaccacccaccttct

SEQ ID NO: 64 - DNA, 22 bp, synthetic construct
  source 1..22: mol_type = other DNA
gcctgtagccgacgtctctcct

SEQ ID NO: 65 - RNA, 20 nt, synthetic construct
  source 1..20: mol_type = other RNA
tggatgagcagcagaaagtt

SEQ ID NO: 66 - RNA, 19 nt, synthetic construct
  source 1..19: mol_type = other RNA
gggccactagggacaggat

SEQ ID NO: 67 - RNA, 20 nt, synthetic construct
  source 1..20: mol_type = other RNA
atgctgaagatgctaccgaa

SEQ ID NO: 68 - RNA, 20 nt, synthetic construct
  source 1..20: mol_type = other RNA
gaggagtgaaggcggcaaaa

SEQ ID NO: 69 - DNA, 198 bp, Homo sapiens
  source 1..198: mol_type = other DNA
ggcgacgcccgtgtttacctgaaagtctcggtcttcgttgaagcgggagaagcggtccgctccggagctgcccttgtcgtcgtcgtccttgtagtcgatgtcgtggtccttgtagtcaccgtcgtggtccttgtagtccatgttgccgccttcactcctctcaggacgacgctctccggttcgctccttccctcctgc

SEQ ID NO: 70 - DNA, 22 bp, synthetic construct
  source 1..22: mol_type = other DNA
gggaaccggggaggggtgggtg

SEQ ID NO: 71 - DNA, 23 bp, synthetic construct
  source 1..23: mol_type = other DNA
agagggctgggagccggagatga

SEQ ID NO: 72 - protein, 47 aa, Homo sapiens
ENIGYQMLMKMGWKEGEGLGSEGQGIKNPVNKGTTVDGAGFGIDRPA

SEQ ID NO: 73 - protein, 48 aa, Homo sapiens
SCNIGHRLLQKHGWKLGQGLGKSLQGRTDPIPIVVKYDVMGMGRMEME

SEQ ID NO: 74 - protein, 49 aa, Homo sapiens
CENKGFALLQKMGYKSGQALGKSGGGIVEPIPLNIKTGKSGIGHEASLK

SEQ ID NO: 75 - protein, 5 aa, synthetic construct
YYYYY

SEQ ID NO: 76 - protein, 5 aa, synthetic construct
RRRRR

SEQ ID NO: 77 - DNA, 79 bp, Homo sapiens
  source 1..79: mol_type = other DNA
gagtgaaggcggcaaaatggcggaccgcttctcccgcttcaacgaagaccgagactttcaggtaaacacgggcgtcgcc

SEQ ID NO: 78 - DNA, 157 bp, synthetic construct
  source 1..157: mol_type = other DNA
gagtgaaggcggcaacatggactacaaggaccacgacggtgactacaaggaccacgacatcgactacaaggacgacgacgacaagggcagctccggagcggaccgcttctcccgcttcaacgaagaccgagactttcaggtaaacacgggcgtcgcc

SEQ ID NO: 79 - protein, 47 aa, synthetic construct
MDYKDHDGDYKDHDIDYKDDDDKGSSGADRFSRFNEDRDFQVNTGVA

SEQ ID NO: 80 - DNA, 26 bp, synthetic construct
  source 1..26: mol_type = other DNA
cacggcgccgttcgnagcatcttccn

SEQ ID NO: 81 - DNA, 26 bp, synthetic construct
  source 1..26: mol_type = other DNA
cacggcgccgttcggtagcatcttca